(19) United States
(12) Patent Application Publication
(10) Pub. No.: US 2009/0142336 A1
(43) Pub. Date: Jun. 4, 2009

(54) METABOLIC REGULATORS AND USES THEREOF

(75) Inventors: Kenneth Walsh, Carlisle, MA (US); Noriyuki Ouchi, Brookline, MA (US); Ling Zeng, Boston, MA (US)

Correspondence Address: RONALD I. EISENSTEIN, 100 SUMMER STREET, NIXON PEABODY LLP, BOSTON, MA, 02110, US

(73) Assignee: TRUSTEES OF BOSTON UNIVERSITY, BOSTON, MA (US)

(21) Appl. No.: 12/280,961

(22) PCT Filed: Feb. 23, 2007
(86) PCT No.: PCT/US07/04793

§ 371 Date: Aug. 27, 2008

Related U.S. Application Data
(60) Provisional application No. 60/777,654, filed on Feb. 28, 2006.

(51) Int. Cl. A61K 39/395; A61K 31/7088; A61K 31/7105; A61K 38/02

(52) U.S. Cl. 424/130.1; 514/44; 514/2

(57) ABSTRACT

The present invention relates to metabolic regulators that affect metabolic function, for example metabolic regulators that affect muscle mass, muscle regeneration, muscle hypertrophy, fat mass, insulin and glucose sensitivity, angiogenesis and cardiovascular function. In particular, the present invention relates to modulating metabolic function by administering an effective amount of a pharmaceutical composition comprising an agent, where the agent activates or inhibits the activity and/or gene expression of the metabolic regulator. The metabolic regulators of the present invention are, for example MSP1 (2160028F08Rik; SEQ ID NO: 16); MSP2 (2310043I08Rik; SEQ ID NO: 17); MSP3 (NM_026754;1 110017116Rik; SEQ ID NO: 1); MSP4 (4732466D17Rik; SEQ BD NO: 18); MSP 5 (NM_024237; 1600015H20Rik; SEQ ID NO: 12); Ins16 (AF_156094; SEQ ID NO: 20). The present invention also provides methods to screen for agents that affect the metabolic regulators of the present invention.

### CROSS REFERENCE TO RELATED APPLICATIONS

This application claims the benefit under 35 U.S.C. 119(e) of U.S. Provisional Patent Application Ser. No. 60/777,654, filed Feb. 28, 2006, the contents of which are herein incorporated by reference in their entirety.

### FIELD OF THE INVENTION

The present invention is directed to metabolic regulators and uses thereof, including therapeutic uses. In particular, the present invention provides uses of metabolic regulators that affect metabolic function, for example metabolic regulators that affect muscle mass, muscle regeneration, muscle hypertrophy, fat mass, insulin and glucose sensitivity, angiogenesis and cardiovascular function. In particular, the present invention relates to modulating metabolic function by administering an effective amount of a pharmaceutical composition comprising an agent, where the agent activates or inhibits the activity and/or gene expression of the metabolic regulator.

### BACKGROUND

Systemic muscle atrophy occurs upon fasting and in a variety of diseases such as cachexia, cancer, AIDS, prolonged bed rest, and diabetes (1) and myscular dystropgy. One strategy for the treatment of atrophy is to induce the pathways normally leading to skeletal muscle hypertrophy.

In particular, a decrease in muscle mass, or atrophy, is associated with various physiological and pathological states. For example, muscle atrophy can result from denervation due to nerve trauma; degenerative, metabolic or inflammatory neuropathy, e.g. Guillian-Barre syndrome; peripheral neuropathy; or nerve damage caused by environmental toxins or drugs. Muscle atrophy may also result from denervation due to a motor neuropathy including, for example, adult motor neuron disease, such as Amyotrophic Lateral Sclerosis (ALS or Lou Gehrig's disease); infantile and juvenile spinal muscular atrophies; and autoimmune motor neuropathy with multifocal conductor block. Muscle atrophy may also result from chronic disease resulting from, for example, paralysis due to stroke or spinal cord injury; skeletal immobilization due to trauma, such as, for example, fracture, ligament or tendon injury, sprain or dislocation; or prolonged bed rest. Metabolic stress or nutritional insufficiency, which may also result in muscle atrophy, include inter alia the cachexia of cancer and other chronic illnesses including AIDS, fasting or rhabdomyolysis, and endocrine disorders such as disorders of the thyroid gland and diabetes. Muscle atrophy may also be due to a muscular dystrophy syndrome such as Duchenne, Becker, myotonic, fascioscapulohumeral, Emery-Dreifuss, oculopharyngeal, scapulohumeral, limb girdle, and congenital types, as well as the dystrophy known as Hereditary Distal Myopathy. Muscle atrophy may also be due to a congenital myopathy, such as benign congenital hypotonia, central core disease, nemalene myopathy, and myotubular (centronuclear) myopathy. Muscle atrophy also occurs during the aging process.

Skeletal muscle hypertrophy plays an important role in normal postnatal development and the adaptive response to physical exercise (2). This process is associated with blood vessel recruitment, such that capillary density is either maintained or increased in the growing muscle (3-6). Conversely, myofiber atrophy that occurs with aging, disuse, and myopathic disease is associated with capillary loss (7, 8). It has been shown that angiogenesis and compensatory muscle hypertrophy are temporally coupled, suggesting that these two processes may be controlled by common regulatory mechanisms (9).

Muscle accumulation inversely correlates with fat mass. For example, skeletal muscle-specific expression of IGF-1 (10), ski (11) or Akt1 (12). However, the hormonal regulatory mechanisms by which skeletal muscle controls lipolysis and fatty acid mobilization and uptake by muscle is poorly understood. Furthermore, proper skeletal function is also important for maintenance of normal glucose metabolism (13-15). Skeletal muscle resistance to insulin-stimulated glucose uptake is the earliest known manifestation of non-insulin-dependent (type 2) diabetes mellitus (16, 17). Increasing skeletal muscle activity can improve insulin sensitivity and prevent the progression from impaired insulin tolerance in most type 2 diabetic patients (18).

Therefore, a useful therapeutic strategy to target these diseases, either individually or together, would be to use molecules that are endogenously secreted by the muscle to increase muscle hypertrophy, angiogenesis, and reduce fat mass and improve insulin sensitivity for the treatment or prevention of various diseases and/or disorders.

### SUMMARY OF THE INVENTION

The invention relates to the discovery of metabolic regulators from muscle for the treatment of a number of pathological conditions including muscle-wasting diseases, insulin-related disorders, obesity, diabetes, tissue ischemia and bone disease, and muscle degenerative diseases.

By using a transgenic mouse model overexpressing Akt, in skeletal muscle, the inventors discovered several muscle secreted proteins, herein termed “metabolic regulators” that affect metabolic function, for example, metabolic regulators that affect muscle mass, muscle hypertrophy, muscle regeneration, insulin sensitivity and glucose sensitivity, angiogenesis and fat mass. The inventors discovered the following metabolic regulators affect metabolic function, for example muscle secreted proteins (MSPs); MSP1, MSP2, MSP3, MSP4, MSP5 and Insulin like protein 6 (Ins16). Accordingly, the present invention provides methods to modulate metabolic function by administering an effective amount of an agent targeting the metabolic regulators of the present invention.

Accordingly, the present invention provides methods to treat disease and/or disorders associated with muscle related diseases, angiogenesis, obesity, glucose intolerance and insulin-dependent disorders and muscle degeneration, the method comprising administering a composition of therapeutically effective amounts of agents of the metabolic regulators of the present invention.

In particular, the present invention provides methods to modulate metabolic function by administering an effective amount of a pharmaceutical composition comprising an agent targeting at least one metabolic regulator of the present invention, for example agents that activate or inhibit the function of metabolic regulators MSP1, MSP2, MSP3, MSP4, MSP5 and Insulin like protein 6 (Ins16). In some embodiments the agent is an agonist that activates the protein and/or increases the gene expression of MSP1, MSP2, MSP3, MSP4, MSP5 and Ins16, and in some embodiments the agent is a polypeptide or nucleic acid encoding MSP1, MSP2, MSP3, MSP4, MSP5 or Ins16 or a homologue or variant or fragment thereof. In alternative embodiments, the agent is an antagonist that inhibits the protein and/or decreases the gene expression of MSP1, MSP2, MSP3, MSP4, MSP5 and Ins16, and in some embodiments the agent is a inhibitory polypeptide, for example dominant negative polypeptide or neutralizing antibody or inhibitory nucleic acid, for example but not limited to antisense nucleic acid and/or RNAi.

The inventors have also discovered MSP3 functions to both modulate angiogenesis and modulate glucose sensitivity, and thus the inventors have discovered that MSP3 functions as a bifunctional myokine to promote blood vessel formation and as a metabolic regulator to increase sensitivity. Accordingly, the present invention relates to the treatment of disorders associated with loss of blood vessels, for example but not limited to ischemia, the method comprising administering an effective amount of an agent that functions as an agonist of MSP3, for example an agent that affects the activity and/or expression of MSP3. One example of such an agonist of MSP3 is, for example but not limited to an agent comprising a nucleic acid encoding MSP3, or a MPS3 protein or fragment or functional derivative thereof or an agonist to MSP3. In an alternative embodiment, the present invention relates to the treatment of disorders associated with and/or insulin intolerance, for example but not limited to, diabetes and obesity, the method comprising administering an effective amount of an agonist of MSP3, for example a nucleic acid encoding MSP3 or a homologue or variant thereof, MSP3 protein or fragment or functional derivative thereof or an agonist to MSP3.

The inventors have also discovered MSP5 functions to modulate angiogenesis and muscle hypertrophy and muscle mass, and therefore the inventors have discovered that MSP5 also functions as a bifunctional myokine to promote blood vessel formation and to promote muscle hypertrophy factor. Accordingly, the present invention related to the treatment of disorders associated with blood vessel loss, the method comprising administering a therapeutically effective amount of an agent that functions as an agonist, for example, and agent that affects the activity and/or expression of a MSP5. One example of such an agent is, for example but not limited to, a nucleic acid encoding MSP5, a MSP5 protein or fragment or functional derivative thereof and/or an agonist to MSP5. In another embodiment, the present invention relates to the treatment of disorders associated with muscle atrophy or where an increase in muscle mass is desired, the method comprising administering an effective amount of an agent that functions as an agonist to MSP5, for example an agent that affects the activity and/or expression of MSP5, for example nucleic acids encoding MSP5 or functional derivatives thereof, MSP5 protein or fragment or functional derivative thereof or an agonist to MSP5.

The inventors have also discovered insulin like protein 6 (Ins16) functions to regulate muscle regeneration by modulating the amount of satellite cell recruitment to muscle myfibrils. Accordingly, the present invention relates to the treatment of disorders associated with muscle degeneration, for example, but not limited to muscular dystrophies, or the treatment of disorders where an increase in muscle mass is desired, for example age related atrophy, the method comprising administering an effective amount of an agent that functions as an agonist, for example an agent that increases the activity and/or expression of Ins16. One example of such an agent, is but not limited to an agonist that is a nucleic acid encoding Ins16 or a homologue or derivative thereof, a Ins16 protein or fragment or functional derivative thereof, or an agonist to Ins16.

In one aspect, the invention provides methods for the treatment of a disease or condition, comprising administering an effective amount of a pharmaceutical composition comprising a nucleic acid encoding MSP1, MSP2, MSP3, MSP4, MSP5 or Ins16 gene, or nucleic acids that encode functional derivatives or variants thereof of the invention, to a subject in need thereof, or a subject at risk for development of that disease or condition. In some embodiments, the pharmaceutical composition comprises an agent that is a protein or polypeptide of MSP1, MSP2, MSP3, MSP4, MSP5 or Ins16 in alternative embodiments, is a fragment or derivative thereof. In another embodiment, the pharmaceutical composition comprises an agent that functions as an agonist, for example activates the gene expression of MSP1, MSP2, MSP3, MSP4, MSP5 or Ins16 or activates a post-transcriptional gene product and/or peptide of MSP1, MSP2, MSP3, MSP4, MSP5 or Ins16.

In some embodiments, the methods of the present invention can be used treat muscle related conditions. In such embodiments where the disease or condition is a muscle related condition or disorder, such as atrophy, the method of the present invention comprises administering an effective amount of an pharmaceutical composition comprising an agent that affects the activity and/or expression of the metabolic regulators of the present invention, for example, MSP1, MSP2, MSP3, MSP4, MSP5 or Ins16, or functional derivatives or variants thereof of the invention, to a subject in need thereof, wherein the muscle-related disease or condition is ameliorated or inhibited. A muscle-related condition or disorder treated by the pharmaceutical compositions of the invention may arise from a number of sources, including for example, but not limited to denervation; degenerative, metabolic or inflammatory neuropathy; infantile and juvenile spinal muscular atrophies; autoimmune motor neuropathy; from chronic disease, including cachexia resulting from cancer, AIDS, fasting or rhabdomyolysis; and from muscular dystrophy syndromes such as Duchenne.

The methods of the present invention are also useful to treat any condition which is results from a deficiency in at least one of the metabolic regulators of the present invention, for example at least one of the following MSP1, MSP2, MSP3, MSP4, MSP5 or Ins16 or which may be improved by increased activity and/or gene expression of MSP1, MSP2, MSP3, MSP4, MSP5 or Ins16, including dwarfism and heart disease, for example, improved heart tissue survival following myocardial infarction.

One aspect of the present invention provides methods for increasing muscle mass in an organism. The present invention provides methods and compositions to treat a subject at risk of developing, or having muscle atrophy, or a subject in need of muscle hypertrophy. In some embodiments, the method comprises administration of an effective amount of a pharmaceutical composition comprising an agent that is an agonist, i.e. an agonist increases the activity and/or increases gene expression of at least one metabolic regulators of the present invention, for example but not limited to, an agent functioning as an agonist that activates MSP5 and/or Ins16 or functional derivatives or homologues thereof, to a subject in need thereof. In some embodiments, the pharmaceutical compositions comprise an agonist of at least one metabolic regulator of the present invention, for example, an agonist of MSP5 and/or Ins16. In an alternative embodiment, the present invention provides a means to treat a subject with, or at risk of muscle atrophy by administering a pharmaceutical composition comprising a nucleic acid encoding at least on metabolic regulator, for example MSP5 (SEQ ID: NO 12) and/or Ins16 (SEQ ID NO:20) or homologues or fragments thereof to the subject. Accordingly, the present invention provides methods to increase muscle growth by increasing muscle hypertrophy or inhibiting atrophy in a subject in need thereof. In an alternative embodiment, the present invention can prevent muscle hypertrophy and reduce muscle mass and/or reduce body weight by administering to a subject an agent that functions as an antagonist, for example an inhibitor of, for example but not limited to an inhibitor of MSP5. Examples of such inhibitors include, for example, a dominant negative form of MSP5, or an inhibitor nucleic acid to MSP5, for example a MSP5 RNAi or MSP5 antisense oligonucleotide.

In another embodiment, the present invention provides methods for modulating muscle regeneration and muscle mass in an organism. In one embodiment, the present invention provides methods and compositions to increase muscle regeneration and/or muscle mass in a subject. For example, the methods of the present invention provide methods and compositions to treat a subject at risk of developing, or having muscle degeneration or a subject in need of muscle regeneration. In such an embodiment, the method comprises administration of an effective amount of a pharmaceutical composition comprising an agent that functions as an agonist, for example but not limited to agents that activate and/or increases the gene expression of at least one metabolic regulator of the present invention, for example but not limited to an agent that activates Ins16 or functional derivatives thereof to a subject in need thereof. In some embodiments, the pharmaceutical compositions comprise an agonist of at least one metabolic regulator of the present invention, for example an agonist of Ins16. In an alternative embodiment, the present invention provides a means to treat a subject with, or at risk of developing muscle degeneration by administering a pharmaceutical composition comprising a nucleic acid encoding at least one metabolic regulator of the present invention, for example but not limited to Ins16 (SEQ ID: NO:20) or a homologue or variant, or fragment thereof to the subject. Accordingly, the present invention provides methods to increase muscle mass for example by increasing satellite cell recruitment and increasing muscle regeneration in a subject in need thereof. In an alternative embodiment, the present invention can be used to prevent excess muscle mass and reduce muscle mass and/or reduce body weight by administering to a subject an agent that functions as an antagonist or inhibitor of, for example Ins16 Such an antagonist is, for example, a dominant negative form of ins16, or an inhibitor nucleic acid to Ins16, for example a Ins16 RNAi or Ins16 antisense oligonucleotide

In yet another embodiment, the present invention relates to methods for modulating glucose and/or insulin sensitivity in an organism. The present invention provides methods and compositions to treat a subject at risk of developing or having insulin and/or glucose insensitivity or a subject in need of glucose regulation or metabolic regulation, for example a subject with an insulin-related disorder or a disorder involving insulin resistance. In such an embodiment, the method comprises administration of an effective amount of a pharmaceutical composition comprising an agent that functions as an agonist, for example increasing activation and/or increasing gene expression of at least one metabolic regulator of the present invention, for example but not limited to an agent that activates MSP3 or functional derivatives thereof to a subject in need thereof. In some embodiments, the pharmaceutical compositions comprise an agonist of a metabolic regulator of the present invention, for example an agonist of MSP3. In an alternative embodiment, the present invention provides a means to treat a subject with, or at risk of an insulin-dependent disorder or obesity by administering a pharmaceutical composition comprising a nucleic acid encoding a metabolic regulator of the present invention, for example a nucleic acid encoding MSP3 (SEQ ID: NO1) or a homologue or variant or fragment thereof to the subject. Accordingly, the present invention provides methods to increase glucose sensitivity and/or increase insulin sensitivity or treat obesity in a subject in need thereof. In an alternative embodiment, the present invention can increase fat mass and/or increase body weight by administering to a subject a pharmaceutical composition comprising an agent that functions as an antagonist, for example an inhibitor of, for example MSP3. Examples of such antagonists include but are no limited to, dominant negative forms of MSP3, or inhibitor nucleic acids of MSP3, for example a MSP3 RNAi or MSP3 antisense oligonucleotide and/or neutralizing antibodies of MSP3.

In yet another embodiment, the present invention relates to methods modulating angiogenesis in an organism. In one embodiment, the present invention provides methods and compositions to increase angiogenesis in a subject. For example, the methods of the present invention provides methods and compositions to treat a subject at risk of developing ischemia or having lack of angiogenesis and/or blood vessel formation, or a subject in need of increased angiogenesis. In such an embodiment, the method comprises administration of an effective amount of a pharmaceutical composition comprising at least one agent that functions as an agonist, for example increases activity and/or increases gene expression of at least one metabolic regulator of the present invention, for example but not limited to an agent that activates MSP3 and/or MSP5 or functional derivatives thereof to a subject in need thereof. In further embodiments, the pharmaceutical composition can comprise agents that are agonists of at least one metabolic regulator of the present invention, for example an agonist of MSP3 and/or MSP5 or homologues thereof. In some embodiments, the method to increase angiogenesis is administration of a pharmaceutical composition comprising a nucleic acid encoding at least one metabolic regulator of the present invention, for example MSP3 (SEQ ID: NO1) and/or MSP5 (SEQ ID NO: 12) or homologues or variants or fragments thereof to the subject. Accordingly, the present invention provides methods to increase angiogenesis in a subject in need thereof.

In an alternative embodiment, the present invention provides methods and compositions to treat a subject in need of reduced angiogenesis or a subject at risk of developing excessive angiogenesis, for example a subject with or at risk of developing cancer. In such an embodiment, the method comprises administration of an effective amount of a pharmaceutical composition comprising an agent that functions as an antagonist or inhibitor to at least one metabolic regulator of the present invention, for example but not limited to an inhibitor of MSP3 and/or an inhibitor of MSP5 or functional derivatives thereof, to a subject in need thereof. Examples of such inhibitors of metabolic regulators useful to inhibit angiogenesis are, for example but not limited to, dominant negative forms of MSP3 and/or MSP5, or inhibitory nucleic acids of MSP3 and/or MSP5, for example MSP3 RNAi or MSP5 RNAi or MSP3 or MSP5 antisense oligonucleotide or neutralizing antibodies of MSP3 and/or MSP5. Accordingly, the present invention provides methods to decrease angiogenesis in a subject in need thereof.

In yet another embodiment, the present invention relates to methods for regulating obesity in an organism. In one embodiment, the present invention provides methods and compositions to reduce and/or treat obesity in a subject. For example, the methods of the present invention provide compositions to treat a subject at risk of developing obesity or an obese subject. In such an embodiment, the method comprises administration of an effective amount of a pharmaceutical composition comprising at least one agent that functions as an agonist, for example an agent that increases activity and/or increase expression of at least one metabolic regulator of the present invention, for example but not limited to an agent that activates MSP3 functional derivatives thereof to a subject in need thereof. In some embodiments, the pharmaceutical compositions comprise an agonist of a metabolic regulator of the present invention, for example an agonist of MSP3. In an alternative embodiment, the present invention provides a means to treat a subject with, or at risk of an insulin-dependent disorder or obesity by administering a pharmaceutical composition comprising a nucleic acid encoding a metabolic regulator or homologue thereof of the present invention, for example a nucleic acid encoding MSP3 (SEQ ID: NO1) or a homologue or variant or fragment thereof to the subject. Accordingly, the present invention provides methods to treat obesity and reduce body weight in a subject in need thereof. In an alternative embodiment, the present invention can increase fat mass and/or increase body weight by administering to a subject an effective amount of a pharmaceutical composition comprising an agent that functions as an antagonist or inhibitor of, for example MSP3, MSP5 and/or Ins16. Example of such antagonists are, for example but not limited to, dominant negative forms of MSP3, MPS5 and Ins16, or inhibitory nucleic acids of MSP3, MPS5 and Ins16, for example a RNAi or antisense oligonucleotide molecules to MSP3, MSP5 and/or Ins16 and/or neutralizing antibodies to MSP3, MSP5 and/or Ins16. Other objects and advantages will become apparent from a review of the ensuing detailed description.

### BRIEF DESCRIPTION OF THE FIGURES

FIGS. 1A-C show generation of skeletal muscle-specific conditional Akt1 TG mice. (FIG. 1A) Schematic illustration of binary TG system. FIG. 1B shows DOX-dependent expression of Akt1 transgene. Top: Temporal profile of DOX treatment. Bottom: Western blot analysis of transgene expression in gastrocnemius muscle. FIG. 1C shows western blot analysis of transgene expression in different tissues.

FIGS. 2A-D shows conditional activation of Akt1 in skeletal muscle caused reversible muscle hypertrophy. FIG. 2A Top shows Temporal profile of DOX treatment. FIG. 2A Bottom shows representative gross appearance of the DTG mice. FIG. 2B Top shows time course of transgene expression. FIG. 2B bottom shows Time course of gastrocnemius muscle weight. Results are presented as mean±SEM (n=4-12 each) *P<0.05 vs. day 0; #P<0.05 vs. day 14. FIG. 2C shows histological analysis. Top: H&E staining of gastrocnemius muscle sections. Scale bars: 100 μm. Bottom, Left: Distribution of mean cross-sectional areas of muscle fibers. Right: Mean cross-sectional areas of muscle fibers. Results are presented as mean±SEM (n=6). *P<0.05 vs. control. FIG. 2D Top shows Representative western blot and histological analysis of gastrocnemius muscle in DTG 2 or 6 weeks after Akt1 activation. Scale bars: 100 μm. Bottom: Gastrocnemius muscle weight and mean cross-sectional areas of muscle fibers. Results are presented as mean±SEM (n=6). *P<0.05 vs. control.

FIGS. 3A-C shows Akt 1-mediated type II muscle growth led to increase peak force output. FIG. 2A shows representative images of gastrocnemius sections from control and DTG mice 2 weeks after Akt1 activation stained with anti-HA antibody and MHC isoform antibodies, for MHC type I, MHC Type IIa and MHC Type IIb, with FIG. 1A showing Akt1 transgene expression induces the growth and hypertrophy of MHC Type IIb fibers (panels) and quantified in the histogram. FIG. 3B shows Forearm grip strength measurements. Results are presented as mean±SEM (n=6)*P<0.05 vs. control. FIG. 3C shows forced treadmill exercise test, with the left graph showing Running time, and the right showing running distance. Results are presented as mean±SEM (n=4)*P<0.05 vs. control.

FIGS. 4A-D show Akt 1-mediated type II muscle growth regressed diet-induced obesity. FIG. 4A Left shows representative gross appearance and ventral view of the control and DTG mice fed HF diet, and FIG. 4A Right shows body weight of control and DTG mice (n=12). FIG. 4B Left shows Representative MRI images of each group of mice which were shown at a level of the right renal pelvis, and FIG. 4B Right shows quantified measurements of total fat volume (n=4). FIG. 4C shows Histological analysis. H&E stained gastrocnemius muscle (Top) and white adipose tissue (Bottom) sections. Scale bars: 200 μm. FIG. 4D shows Gastrocnemius muscle and inguinal fat pad weight. Results are presented as mean±SEM (n=6). *P<0.05.

FIGS. 5A-D show Akt1-mediated type II muscle growth improved diet-induced severe insulin resistance. FIG. 5A shows blood glucose levels in Fasting (left) and fed (right) period. FIG. 5B shows fasting serum insulin levels. (FIG. 4C) Glucose tolerance tests. (FIG. 5D) Glucose uptake in vivo skeletal muscle. Results are presented as mean±SEM (n=8). *P<0.05 vs. HF diet fed control mice.

FIGS. 6A-C shows Akt1-mediated type IIb muscle growth led to increased energy consumption independent of food intake or activity level. (FIG. 6A) Food consumption was measured over 6 weeks after Akt1 activation in skeletal muscle (n=12 in each group). (FIG. 6B) Ambulatory activity levels (n=6 in each group). (FIG. 6C) left: 02 consumption (V02), right: respiratory exchange ratio measured by metabolic measuring system for 24 hours without food in control (white bars) and DTG (black bars) mice 4 weeks after DOX treatment (n=6 in each group). Results are presented as mean±SEM. DTG=MyoMouse.

FIGS. 7A-D shows Akt1-mediated type II muscle growth increased lipid oxidation in liver. FIG. 7A: Histological analysis. Oil red-O stained liver sections. Scale bars: 200 μm. FIG. 7B shows Total fatty acid β-oxidation of palmitic acid in liver. FIG. 7C shows fasting serum ketone body levels. FIG. 7D shows quantitative real-time PCR analysis in liver. Results are presented as mean±SEM (n=6).

FIG. 8 shows an alternative approach for the isolation of myokines: Myogenic cell lines transduced with an activated form of Akt.

FIG. 9 shows Akt1 induction by administration of Dox in drinking water leads predominantly to the hypertrophy of Type IIb muscle fibers that are characterized as glycolytic/fast twitch. Less growth of oxidative/fast twitch muscle fibers (Type I, Type IIa) is evident.

FIG. 10A-F shows Akt-mediated type IIb muscle hypertrophy increased lipid oxidation in liver, but not muscle. FIG. 10A shows relative mRNA expression associated with fatty acid oxidation and mitochondrial biogenesis in gastrocnemial skeletal muscle (n=4 in each group. *P<0.05 vs. HF/HS diet fed control. ˜0.05 vs. normal diet fed control). FIG. 10B is Histological analysis. Oil red-O stained liver sections. Scale bars: 100 pm. FIG. 10C is Total fatty acid ˜3-oxidation of palmitic acid in liver (n=8 in each group). FIG. 10D s Expression of genes associated with fatty acid ˜3-oxidation in liver (n=4 in each group). (E) Serum (left) and urine (right) ketone body levels (n=9 to 12 in each group). FIG. 10F shows Serum lactate levels (n=6 in each group). Results are presented as mean±SEM. DTG=MyoMouse.

FIG. 11 shows evidence for satellite cell proliferation following Akt1 activation in MyoMice. FIG. 11A shows evidence for satellite cell proliferation at 2 weeks after transgene activation. BrdU incorporation into DNA was evident in histological sections of MyoMice 2, 4, 6, 8 and 10 weeks (w) after activation of Akt1, but not in control (cont) mice. Evidence for increased numbers of MyoD-positive satellite cells in MyoMice 2 weeks after transgene induction was also detected (not shown)

FIG. 12 is a schematic of tissues for differential gene expression analysis. Microarray analyses on muscle, liver and adipose tissues of diet-induced obese Akt1-mediated skeletal muscle expression (MyoMice) before and after Akt transgene expression. Analysis of such tissues enables identification of potential receptors and proteins secreted from liver and adipose tissue in response to skeletal muscle growth.

FIGS. 13A-C shows the effect of adenovirus expressing MSP3 on ischemia-induced angiogenic response in wild-type mice. FIG. 13B shows a mouse hindlimb ischemia model, and Adenovirus-expressed MSP3 promotes blood vessel growth in the ischemic limb as monitored by Laser Doppler analysis (FIG. 13B) on legs and feet immediately before surgery and on postoperative days 0, 3, 7, 14, and 28 as illustrated in FIG. 11A. FIG. 11C shows intramuscular injection of an adenoviral vector expressing MSP3, but not MSP6 (FGF-21) or β-galactosidase, stimulates reperfusion in ischemic hindlimb of mice as assessed by laser Doppler analysis.

FIG. 14 shows the effect of Adv-MSP3 on capillary density in WT mice. Adenovirus-expressed clone 2 (MSP3) promotes microvessel formation as assessed by CD31-staining in histological section from ischemic limb. An adenoviral vector expressing FGF21 does not display this activity.

FIGS. 15A-D shows glucose tolerance test. MSP3 is a candidate metabolic regulator. Intramuscular injection of Adeno-MSP3 improves glucose sensitivity in a diet-induced obesity mouse model. FIGS. 15A and 15B shows adenovirus-encoded MSP3 appears functionally equivalent to adenovirus-delivered FGF-21 (also known as MSP6). FIGS. 15C and 15D shows MSP3 improves glucose sensitivity and metabolic response, which is not observed for other MSPS; MSP5, MSP2, MSP4 and MSP1. β-gal is the negative control.

FIG. 16 shows the full-length nucleotide sequence of MSP3. Nucleotide sequence of MSP3 showing “long” (SEQ ID NO: 1) and “short” (SEQ ID NO:2) alternatively-spliced forms.

FIG. 17 shows the position of MSP3 long (SEQ ID NO: 1) and short (SEQ ID NO:2) and on chromosome 2.

FIG. 18 shows the alignments of MSP3 amino acid sequences between mouse (SEQ ID NO: 3), rat (SEQ ID NO: 4), and human (SEQ ID NO: 5). Amino acid sequence identity between mouse and rat is 94% and the sequence identity between mouse and human is 79%. The boxed area is the predicted signal sequence.

FIG. 19 shows PCR primers for detecting total expression of MSP3 (i.e for detecting both the long (SEQ ID NO: 1) and short (SEQ ID NO:2) isoforms of MSP3. The location of the forward primer 3 is SEQ ID NO: 10, and the reverse primer 3 is SEQ ID NO: 11) are shown on the SEQ ID NO: 1 and SEQ ID NO:2.

FIGS. 20A-B show tissue-specific expression profile of MSP3 in adult mouse tissues. FIG. 20A shows expression profile of total MSP3 (combined long and short forms) by RT-PCR. Expression in heart, brain, lung, thymus, lymph node, eye and skeletal muscle. Upregulation by Akt expression in C2C12 myogenic cells. FIG. 20B shows expression profile of MSP3 long and short forms in adult mouse tissues by RT-PCR.

FIG. 21 shows alternative splice isoform specific PCR Primers: Design of PCR Primers to differentially detect long and short forms of MSP3. Location and design of primers to detect long and short forms of (MSP3) clone 2 are shown, with the positions of Forward Primer 1 (SEQ ID NO:6), Forward Primer 2 (SEQ ID NO:8), Reverse Primer 1 (SEQ ID NO:9) and Reverse Primer 2 (SEQ ID NO:10) on the long form (SEQ ID NO: 1) and short form (SEQ ID NO:2) of MSP3 shown.

FIGS. 22A-D shows MSP5 promotes angiogenesis in ischemic hind limb repair as shown by FIG. 22D by laser Dopper analysis. Effect of adenovirus expressing MSP5 (clone 5) on ischemia-induced angiogenic response in wild-type mice. FIG. 22D shows intramuscular injection of an adenoviral vector expressing MSP5, but not MSP1 or β-galactosidase, stimulates reperfusion in ischemic hindlimb of mice as assessed by laser Doppler analysis.

FIGS. 23A-B shows MSP5 promotes myofiber hypertrophy. FIG. 23A outline the protocol to assess MSP5-mediated hypertrophy of C2C12 myocytes in vitro was done by transfecting adenovirus expressing MSP5 or MSP3 or MyrAkt or β-gal 4 days after differentiation of C2C13 myocytes, and FIG. 23B shows the morphology of cells 4 days post-transfection. FIG. 23C shows quantitative analysis of myofiber width 4 days after transfection with Adv-expressing MSP5, MSP3, MyrAkt or β-gal (as shown in FIG. 23B) was examined microscopically. Transduction with MSP5 or myrAkt leads to detectable increases in myotube size, but adenoviral vectors expressing MSP3 or β-galactosidase has no effect.

FIG. 24 shows transduction with adenoviral vectors expressing MSP5 or myrAkt1 promotes 3H-leucine incorporation into protein as compared to baseline incorporation in the absence of virus or C2C12 cells transfected with Adenovirus expressing i-gal or MSP3. Representative results from duplicate experiments (left and right panels) is shown.

FIG. 25 shows MSP5 transfected C2C12 cells promote VEGF expression. Transduction of C2C12 cells with adenoviral vectors expressing MSP5 or myrAkt1, but not MSP3 (clone 2), activate VEGF expression in C2C12 cells.

FIGS. 26A-B shows Insulin-like 6 is regulated by Akt in muscle in vitro (FIG. 26B) and in vivo (FIG. 26A). FIG. 26A shows Insulin-like 6 transcript is dramatically upregulated 24-fold in MyoMice 2 weeks after transgene induction and FIG. 26B shows a 10-fold in C2C12 cells following transduction with Adeno-myrAkt1.

FIGS. 27A-D show that other relaxin family members, such as Ins13, Ins15, relaxin, Ins17 (relaxin 3) are not regulated following Akt transgene induction in MyoMice, as determined by RT-PCR of transcript expression levels.

FIGS. 28A-B shows Insulin-like 6 (Ins16) transcript is upregulated during muscle regeneration following cardiotoxin administration to tibialis anterius (TA) muscle. FIG. 28A shows Akt is also upregulated by this injury, whereas VEGF-A transcript is downregulated (upper panel). FIG. 28B shows other relaxin family members, Ins13, Ins15, relaxin, Ins17 (relaxin 3) are not regulated in cardiotoxin-injured mouse muscle (bottom panel).

FIGS. 29A-D shows Adenovirus-expressing insulin-like 6 (Ins16) does not affect C2C12 differentiation or hypertrophy. FIG. 29A shows C2C12 cells were transfected with Adv-Ins16 or βgal (Gal) at 240 multiplicities of infection (MOI) and morphology, and FIG. 29B shows the number of multi-nucleated myotubes, and FIG. 29E shows myotube width is not affected with transduction with Adv-Ins16. FIG. 29C shows Creatine kinase levels and FIG. 29D shows Leucine incorporation was also compared in C2C12 cells transfected with Adv-Ins16 or Adv-βgal control (Gal).

FIGS. 30A-B shows adenovirus-expressing insulin-like 6 stimulated the proliferation of rat skeletal muscle satellite cells. FIG. 30A shows Thymidine (³H-thymidine) incorporation is increased in Adv-Ins16 transfected cells compared to β-gal control transfected cells. FIG. 30 shows Western blot (WB) shows activation of satellite cell proliferation is accompanied by an increase in Rb protein (p-Rb) proliferation.

FIGS. 31A-B shows Ins16 facilitates TA muscle regeneration after cardiotoxin (CTX) injury. FIG. 31A shows administration of Adeno-Ins16 4 days after cardiotoxin administration improves tibialis arterius (TA) muscle regeneration compared to β-gal control. Improved regeneration is most notable at 7 and 14 days in histological sections (FIG. 31A). FIG. 31B shows at 7 days Ins16 overexpression repressed creatine kinase release into sera (lower left panel) which was not observed at 14 days (lower right panel).

FIG. 32 is similar to FIG. 31A, where administration of Adeno-Ins16 3 days after cardiotoxin (CTX) administration, Ins16 significantly promotes muscle regeneration of tibialis arterius (TA) 1 week following injury compared to β-gal control.

FIGS. 33A-D shows Ins16 reduces expression of TNFα and TNFβ1 and promotes collagen3 expression. Administration of Adeno-Ins16 results in a 200-fold increase in Ins16 expression (FIG. 33A) in the muscle, and reduces TNFα (0.2 fold, p<0.03)(FIG. 33B), and TNFβ1 (0.9 fold, p>0.8) (FIG. 33D) and increases collagen 3 (1.8 fold, p>0.6) (FIG. 33D C) in muscle after injury. Ins16 and TNFα are 1 week post Adv-Ins16 injection (n=2)

### DETAILED DESCRIPTION OF THE INVENTION

### General Description

The present invention is based on the discovery that growing skeletal muscle secretes metabolic regulators that affect muscle growth, angiogenesis, obesity, insulin sensitivity, glucose sensitivity, body weight, fat mass, muscle mass and cardiovascular function. The metabolic regulators discovered by the inventors are, for example MSP1, MSP2, MSP3, MSP4, MSP5 and Ins16. Accordingly, the present invention provides methods to modulate metabolic function by administering effective amounts of agents that activate or inhibit such metabolic regulators, for example MSP1, MSP2, MSP3, MSP4, MSP5 or Ins16, which is useful for the treatment of a variety of diseases and disorders, for example but not limited to, disorders associated with muscle growth, muscle degeneration, muscle atrophy, angiogenesis, obesity, insulin-dependent diseases and cardiovascular function.

### DEFINITIONS

Unless defined herein, terms used herein have their ordinary meanings, and can be further understood in the context of the specification.

A “transgenic animal” (e.g., a mouse or rat) is an animal having in some or all of its cells a transgene, which transgene was introduced into the animal or an ancestor of the animal at a prenatal, e.g., an embryonic stage. A “transgene” is a DNA that is integrated into the genome of a cell from which a transgenic animal develops.

As used herein, the term “operatively linked,” refers to a functional relationship between two or more nucleic acid (e.g., DNA) segments: for example, a promoter or enhancer is operatively linked to a coding sequence if it stimulates the transcription of the sequence in an appropriate host cell or other expression system. Generally, sequences that are operatively linked are contiguous. However, enhancers need not be located in close proximity to the coding sequences whose transcription they enhance. Furthermore, a gene transcribed from a promoter regulated in trans by a factor transcribed by a second promoter may be said to be operatively linked to the second promoter. In such a case, transcription of the first gene is said to be operatively linked to the first promoter and is also said to be operatively linked to the second promoter.

As used herein, “muscle” or “muscle cell” refers to any cell that contributes to muscle tissue. Myoblasts, satellite cells, myotubes, and myofibril tissues are all included in the term “muscle cells”. Muscle cells may include those within skeletal, cardiac and smooth muscles.

The term “agent” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, or any organic or inorganic molecule, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof. For example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

As used herein, the term “antibody” means an immunoglobulin molecule or a fragment of an immunoglobulin molecule having the ability to specifically bind to a particular antigen. Antibodies are well known to those of ordinary skill in the science of immunology. As used herein, the term “antibody” means not only intact antibody molecules but also fragments of antibody molecules retaining antigen binding ability. Such fragments are also well known in the art and are regularly employed both in vitro and in vivo. In particular, as used herein, the term “antibody” means not only intact immunoglobulin molecules of any isotype (IgA, IgG, IgE, IgD, IgM) but also the well-known active fragments F(ab′) (2), Fab, Fv, scFv, Fd, V (H) and V (L). For antibody fragments, see, for example “Immunochemistry in Practice” (Johnstone and Thorpe, eds., 1996; Blackwell Science), p. 69.

As used herein, a “factor associated with muscle growth” refers to a gene or gene product identified by the methods of the present invention. The gene products may be proteins, e.g., secreted proteins, membrane bound proteins, etc. Alternatively, the gene products may be functional RNAs, e.g., microRNAs, ribozymes, etc.

The term “nucleic acid” is well known in the art. A “nucleic acid” as used herein will generally refer to a molecule (i.e., strand) of DNA, RNA or a derivative or analog thereof, comprising a nucleobase. A nucleobase includes, for example, a naturally occurring purine or pyrimidine base found in DNA (e.g. an adenine “A,” a guanine “G.” a thymine “T” or a cytosine “C”) or RNA (e.g. an A, a G. an uracil “U” or a C). The term “nucleic acid” encompasses the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. The term “nucleic acid” also refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. The terms “polynucleotide sequence” and “nucleotide sequence” are also used interchangeably herein.

As used herein, the term “gene” refers to a nucleic acid comprising an open reading frame encoding a polypeptide, including both exon and (optionally) intron sequences. A “gene” refers to coding sequence of a gene product, as well as non-coding regions of the gene product, including 5′UTR and 3′UTR regions, introns and the promoter of the gene product. These definitions generally refer to a single-stranded molecule, but in specific embodiments will also encompass an additional strand that is partially, substantially or fully complementary to the single-stranded molecule. Thus, a nucleic acid may encompass a double-stranded molecule or a double-stranded molecule that comprises one or more complementary strand(s) or “complement(s)” of a particular sequence comprising a molecule. As used herein, a single stranded nucleic acid may be denoted by the prefix “ss”, a double stranded nucleic acid by the prefix “ds”, and a triple stranded nucleic acid by the prefix “is.” The term “gene” refers to the segment of DNA involved in producing a polypeptide chain, it includes regions preceding and following the coding region as well as intervening sequences (introns) between individual coding segments (exons). A “promoter” is a region of a nucleic acid sequence at which initiation and rate of transcription are controlled. It may contain elements at which regulatory proteins and molecules may bind, such as RNA polymerase and other transcription factors, to initiate the specific transcription of a nucleic acid sequence. The term “enhancer” refers to a cis-acting regulatory sequence involved in the transcriptional activation of a nucleic acid sequence. An enhancer can function in either orientation and may be upstream or downstream of the promoter.

As used herein, the term “gene product(s)” is used to refer to include RNA transcribed from a gene, or a polypeptide encoded by a gene or translated from RNA.

The terms “polypeptide” and “protein” are used interchangeably to refer to a polymer of amino acid residues, and are not limited to a minimum length. Thus, peptides, oligopeptides, dimers, multimers, and the like, whether produced biologically, recombinantly, or synthetically and whether composed of naturally occurring or non-naturally occurring amino acids, are included within the definition. Both full-length proteins and fragments thereof are encompassed by the definition. The terms also include co-translational (e.g., signal peptide cleavage) and post-translational modifications of the polypeptide, such as, for example, dissulfide-bond formation, glycosylation, acetylation, phosphorylation, proteolytic cleavage (e.g., cleavage by furins or metalloproteases), and the like. Furthermore, for purposes of the present invention, a “polypeptide” refers to a protein that includes modifications, such as deletions, additions, and substitutions (generally conservative in nature as would be known to a person in the art), to the native sequence, as long as the protein maintains the desired activity. These modifications may be deliberate, as through site-directed mutagenesis, or may be accidental, such as through mutations of hosts that produce the proteins, or errors due to PCR amplification or other recombinant DNA methods. Recombinant, as used herein to describe a nucleic acid molecule, means a polynucleotide of genomic, cDNA, viral, semisynthetic, and/or synthetic origin, which, by virtue of its origin or manipulation, is not associated with all or a portion of the polynucleotide with which it is associated in nature. The term recombinant as used with respect to a protein or polypeptide, means a polypeptide produced by expression of a recombinant polynucleotide. The term recombinant as used with respect to a host cell means a host cell into which a recombinant polynucleotide has been introduced.

The term “protein fragment” is meant to include both synthetic and naturally-occurring amino acid sequences derivable from the naturally occurring amino acid sequence of the metabolic regulators of the present invention, for example MSP1, MSP2, MSP3, MSP4, MSP5 or Ins16. The protein is said to be “derivable from the naturally-occurring amino acid sequence of MSP1, MSP2, MSP3, MSP4, MSP5 or Ins16” if it can be obtained by fragmenting the naturally-occurring chosen sequence of MSP1, MSP2, MSP3, MSP4, MSP5 or Ins16, or if it can be synthesized based upon a knowledge of the sequence of the naturally occurring amino acid sequence or of the genetic material (DNA or RNA) which encodes this sequence.

As used herein, a “positive regulator of muscle growth” refers to a gene and/or gene product where its expression results in muscle growth, and lack of its expression results in no muscle growth.

As used herein, a “negative regulator of muscle growth” refers to a gene and/or gene product where its expression results in no muscle growth, and lack of its expression results in muscle growth.

The term a “dominant negative form” of a molecule, is a structurally altered protein that exerts the opposite phenotypic action on a cell relative to the wild-type protein. For example a dominant negative form of MSP3, is a variant of MSP3 that is capable of inhibiting normal signaling of that molecule.

The term “functional derivative” and “mimetic” are used interchangeably, and refers to a compound which possess a biological activity (either functional or structural) that is substantially similar to a biological activity of the entity or molecule its is a functional derivative of. The term functional derivative is intended to include the fragments, variants, analogues or chemical derivatives of a molecule.

The term “functional derivatives” is intended to include the “fragments,” “variants,” “analogs,” or “chemical derivatives” of a molecule. A “fragment” of a molecule, is meant to refer to any polypeptide subset of the molecule. Fragments of a metabolic regulator, for example MSP1, MSP2, MSP3, MSP4, MSP5 or Ins16 which have the activity and which are soluble (i.e not membrane bound) are also encompassed for use in the present invention. A “variant” of a molecule MSP1, MSP2, MSP3, MSP4, MSP5 or Ins16 is meant to refer to a molecule substantially similar in structure and function to either the entire molecule, or to a fragment thereof. A molecule is said to be “substantially similar” to another molecule if both molecules have substantially similar structures or if both molecules possess a similar biological activity. Thus, provided that two molecules possess a similar activity, they are considered variants as that term is used herein even if the structure of one of the molecules not found in the other, or if the sequence of amino acid residues is not identical. An “analog” of a molecule such as MSP1, MSP2, MSP3, MSP4, MSP5 or Ins16 is meant to refer to a molecule substantially similar in function to either the entire molecule or to a fragment thereof. As used herein, a molecule is said to be a “chemical derivative” of another molecule when it contains additional chemical moieties not normally a part of the molecule. Such moieties can improve the molecule's solubility, absorption, biological half life, etc. The moieties can alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., MackPubl., Easton, Pa.(1990).

The terms “subject” and “individual” are used interchangeably herein, and refer to an animal, for example a human, to whom treatment, including prophylactic treatment, with the pharmaceutical composition according to the present invention, is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. The term does not denote a particular age or sex. Thus, adult and newborn subjects, as well as fetuses, whether male or female, are intended to be covered. Examples of subjects include humans, dogs, cats, cows, goats, and mice. The term subject is further intended to include transgenic species.

The term “tissue” is intended to include intact cells, blood, blood preparations such as plasma and serum, bones, joints, muscles, smooth muscles, and organs.

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, amlady, disorder, sickness, illness, complaint, inderdisposion, affection.

A “composition” or “pharmaceutical composition” are used interchangeably herein refers to a composition that usually contains an excipient, such as a pharmaceutically acceptable carrier that is conventional in the art and that is suitable for administration to cells. The cells may be part of a subject, for example for therapeutic, diagnostic, or prophylactic purposes. The cells may also be cultured, for example cells as part of an assay for screening potential pharmaceutical compositions, and the cells may be part of a transgenic animal for research purposes. The composition can also be a cell culture, in which a polypeptide or polynucleotide encoding a metabolic regulator of the present invention is present in the cells and/or in the culture medium. In addition, compositions for topical (e.g., oral mucosa, respiratory mucosa) and/or oral administration can form solutions, suspensions, tablets, pills, capsules, sustained-release formulations, oral rinses, or powders, as known in the art and described herein. The compositions also can include stabilizers and preservatives. For examples of carriers, stabilizers and adjuvants, University of the Sciences in Philadelphia (2005) Remington: The Science and Practice of Pharmacy with Facts and Comparisons, 21 st Ed.

The term “effective amount” as used herein refers to the amount of therapeutic agent of pharmaceutical composition to alleviate at least one or some of the symptoms of the disease or disorder.

An “insulin-resistant disorder” is a disease, condition, or disorder resulting from a failure of the normal metabolic response of peripheral tissues (insensitivity) to the action of exogenous insulin, i.e., it is a condition where the presence of insulin produces a subnormal biological response. In clinical terms, insulin 15 resistance is present when normal or elevated blood glucose levels persist in the face of normal or elevated levels of insulin. It represents, in essence, a glycogen synthesis inhibition, by which either basal or insulin stimulated glycogen synthesis, or both, are reduced below normal levels. Insulin resistance plays a major role in Type 2 diabetes, as demonstrated by the fact that the hyperglycemia present in Type 2 diabetes can sometimes be reversed by diet or weight loss sufficient, apparently, to restore the sensitivity of peripheral 20 tissues to insulin. The term includes abnormal glucose tolerance, as well as the many disorders in which insulin resistance plays a key role, such as obesity, diabetes mellitus, ovarian hyperandrogenism, and hypertension. “Diabetes mellitus” refers to a state of chronic hyperglycemia, i.e., excess sugar in the blood, consequent upon a relative or absolute lack of insulin action. There are three basic types of diabetes mellitus, 25 type I or insulin-dependent diabetes mellitus (IDDM), type II or non-insulin-dependent diabetes mellitus (NIDDM), and type A insulin resistance, although type A is relatively rare. Patients with either type I or type II diabetes can become insensitive to the effects of exogenous insulin through a variety of mechanisms. Type A insulin resistance results from either mutations in the insulin receptor gene or defects in post-receptor sites of action critical for glucose metabolism. Diabetic subjects can be easily recognized by the physician, and are 30 characterized by hyperglycemia, impaired glucose tolerance, glycosylated hemoglobin and, in some instances, ketoacidosis associated with trauma or illness.

The term “Non-insulin dependent diabetes mellitus” or “NIDDM” refers to Type II diabetes. NIDDM patients have an abnormally high blood glucose concentration when fasting and delayed cellular uptake of glucose following meals or after a diagnostic test known as the glucose tolerance test. NIDDM is diagnosed based on 35 recognized criteria (American Diabetes Association, Physician's Guide to Insulin-Dependent (Type I) Diabetes, 1988; American Diabetes Association, Physician's Guide to Non-Insulin-Dependent (Type II) Diabetes, 1988).

The term “cancer”, as used herein refers to a cellular proliferative disease in a human or animal subject. The term “tumor” or “tumor cell” used interchangeably herein refers to the tissue mass or tissue type or cell type that is undergoing uncontrolled proliferation.

The term “antagonist” or “inhibitor” are used interchangeably herein, refers to any agent or entity capable of inhibiting or suppressing the expression or activity of a protein, polypeptide portion thereof, or polynucleotide. Thus, the antagonist may operate to prevent transcription, translation, post-transcriptional or post-translational processing or otherwise inhibit the activity of the protein, polypeptide or polynucleotide in any way, via either direct of indirect action. The antagonist may for example be a nucleic acid, peptide, or any other suitable chemical compound or molecule or any combination of these. Additionally, it will be understood that in indirectly impairing the activity of a protein, polypeptide of polynucleotide, the antagonist may affect the activity of the cellular molecules which may in turn act as regulators or the protein, polypeptide or polynucleotide itself. Similarly, the antagonist may affect the activity of molecules which are themselves subject to the regulation or modulation by the protein, polypeptide of polynucleotide.

The term “inhibiting” as used herein does not necessarily mean complete inhibition of expression and/or activity. Rather, expression or activity of the protein, polypeptide or polynucleotide or nucleic acid is inhibited to an extent, and/or for a time, sufficient to produce the desired effect.

The term “agonist” refers to any agent or entity capable of activating or enhancing the expression or activity of a protein, polypeptide portion thereof, or polynucleotide. Thus, an agonist may operate to promote gene expression, for example promote gene transcription, translation, post-transcriptional or post-translational processing or otherwise activate the activity of the protein, polypeptide or polynucleotide in any way, via either direct or indirect action. An agonist may for example be a nucleic acid, peptide, or any other suitable chemical compound or molecule or any combination of these. Additionally, it will be understood that in indirectly promoting the activity of a protein, polypeptide of polynucleotide, an agonist may affect the activity of the cellular molecules which may in turn act as regulators or the protein, polypeptide or polynucleotide itself. Similarly, an agonist may affect the activity of molecules which are themselves subject to the regulation or modulation by the protein, polypeptide of polynucleotide. An agonist also refers to any agent that is capable of causing an increase in the activity of a gene and/or gene product in a cell, whether it was present in the cell or absent in the cell prior to adding such an agent. For example, an agent that activates MSP1 or an agonist of MSP1 is an agent that can activate the expression of MSP1 nucleic acid already present in a cell, or an agent can be a nucleic acid encoding MSP1 or a functional derivative thereof, or an agent can be a polypeptide of MSP1, regardless of whether MSP1 already present in the cell, or an agent can be a MSP1 mimetic or functional derivative, for example an analogue of MSP1.

The term “activating” or “activates” are used interchangeably herein, refers to the general increase in activity of a protein, polypeptide portion thereof, or polynucleotide or a metabolic regulator of the present invention. Activation does not necessarily mean complete activation of expression and/or activity of the metabolic regulator, rather, a general or total increase in the expression or activity of the protein, polypeptide or polynucleotide that is activated to an extent, and/or for a time, sufficient to produce the desired effect.

The term “entity” refers to any structural molecule or combination of molecules.

The term “RNAi” as used herein refers to RNA interference (RNAi) a RNA-based molecule that inhibits gene expression. RNAi refers to a means of selective post-transcriptional gene silencing by destruction of specific mRNA by small interfering RNA molecules (siRNA). The siRNA is typically generated by cleavage of double stranded RNA, where one strand is identical to the message to be inactivated.

The term “shRNA” as used herein refers to short hairpin RNA which functions as RNAi and/or siRNA species but differs in that shRNAi species are double stranded hairpin-like structure for increased stability.

The term “antibody” is meant to be an immunoglobulin protein that is capable of binding an antigen. Antibody as used herein is meant to include antibody fragments, e.g. F(ab′)2, Fab′, Fab, capable of binding the antigen or antigenic fragment of interest. The term “humanized antibody” is used herein to describe complete antibody molecules, i.e. composed of two complete light chains and two complete heavy chains, as well as antibodies consisting only of antibody fragments, e.g. Fab, Fab′, F(ab′)2, and Fv, wherein the CDRs are derived from a non-human source and the remaining portion of the Ig molecule or fragment thereof is derived from a human antibody, preferably produced from a nucleic acid sequence encoding a human antibody. The terms “human antibody” and “humanized antibody” are used herein to describe an antibody of which all portions of the antibody molecule are derived from a nucleic acid sequence encoding a human antibody. Such human antibodies are most desirable for use in antibody therapies, as such antibodies would elicit little or no immune response in the human patient.

The term “chimeric antibody” is used herein to describe an antibody molecule as well as antibody fragments, as described above in the definition of the term “humanized antibody.” The term “chimeric antibody” encompasses humanized antibodies. Chimeric antibodies have at least one portion of a heavy or light chain amino acid sequence derived from a first mammalian species and another portion of the heavy or light chain amino acid sequence derived from a second, different mammalian species.

The term “cell” used herein refers to any cell, prokaryotic or eukaryotic, including plant, yeast, worm, insect and mammalian. Mammalian cells include, without limitation; primate, human and a cell from any animal of interest, including without limitation; mouse, hamster, rabbit, dog, cat, domestic animals, such as equine, bovine, murine, ovine, canine, feline, etc. The cells may be a wide variety of tissue types without limitation such as; muscle cells, liver cells, hepatic cells, adipose cells, hematopoietic, neural, mesenchymal, cutaneous, mucosal, strornal, muscle, spleen, reticuloendothelial, epithelial, endothelial, hepatic, kidney, gastrointestinal, pulmonary, T-cells etc. Stem cells, embryonic stem (ES) cells, ES-derived cells and stem cell progenitors are also included, including without limitation, hematopoeitic, neural, stromal, muscle, cardiovascular, hepatic, pulmonary, gastrointestinal stem cells, etc. Yeast cells may also be used as cells in this invention. Cells also refer not to a particular subject cell but to the progeny or potential progeny of such a cell because of certain modifications or environmental influences, for example differentiation, such that the progeny mat not, in fact be identical to the parent cell, but are still included in the scope of the invention.

The cells used in the invention can also be cultured cells, e.g. in vitro or ex vivo. For example, cells cultured in vitro in a culture medium. Alternatively, for ex vivo cultured cells, cells can be obtained from a subject, where the subject is healthy and/or affected with a disease. Cells can be obtained, as a non-limiting example, by biopsy or other surgical means know to those skilled in the art. Cells used in the invention can present in a subject, e.g. in vivo. For the invention on use on in vivo cells, the cell is preferably found in a subject and display characteristics of the disease, disorder or malignancy pathology

As used herein, the term “treating” includes reducing or alleviating at least one adverse effect or symptom of a condition, disease or disorder associated with inappropriate proliferation, for example cancer.

As used herein, the terms “administering,” and “introducing” are used interchangeably herein and refer to the placement of the agents of metabolic regulators of the present invention into a subject by a method or route which results in at least partial localization of the agents of metabolic regulators at a desired site. The compounds of the present invention can be administered by any appropriate route which results in an effective treatment in the subject.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration of cardiovascular stem cells and/or their progeny and/or compound and/or other material other than directly into the central nervous system, such that it enters the animal's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

The term “regeneration” means regrowth of a cell population, organ or tissue, and in some embodiments after disease or trauma.

The term “vectors” used interchangeably with “plasmid” refer to a nucleic acid molecule capable of transporting another nucleic acid to which it has been linked. Vectors capable of directing the expression of genes and/or nucleic acid sequence to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. Other expression vectors can be used in different embodiments of the invention, for example, but are not limited to, plasmids, episomes, bacteriophages or viral vectors, and such vectors may integrate into the host's genome or replicate autonomously in the particular cell. Other forms of expression vectors known by those skilled in the art which serve the equivalent functions can also be used. Expression vectors comprise expression vectors for stable or transient expression encoding the DNA.

The term “viral vectors” refers to the use as viruses, or virus-associated vectors as carriers of the nucleic acid construct into the cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cells genome. The constructs may include viral sequences for transfection, if desired. Alternatively, the construct may be incorporated into vectors capable of episomal replication, e.g EPV and EBV vectors.

As used herein, a “promoter” or “promoter region” or “promoter element” used interchangeably herein, refers to a segment of a nucleic acid sequence, typically but not limited to DNA or RNA or analogues thereof, that controls the transcription of the nucleic acid sequence to which it is operatively linked. The promoter region includes specific sequences that are sufficient for RNA polymerase recognition, binding and transcription initiation. This portion of the promoter region is referred to as the promoter. In addition, the promoter region includes sequences which modulate this recognition, binding and transcription initiation activity of RNA polymerase. These sequences may be cis-acting or may be responsive to trans-acting factors. Promoters, depending upon the nature of the regulation may be constitutive or regulated.

The term “regulatory sequences” is used interchangeably with “regulatory elements” herein refers element to a segment of nucleic acid, typically but not limited to DNA or RNA or analogues thereof, that modulates the transcription of the nucleic acid sequence to which it is operatively linked, and thus act as transcriptional modulators. Regulatory sequences modulate the expression of gene and/or nucleic acid sequence to which they are operatively linked. Regulatory sequence often comprise “regulatory elements” which are nucleic acid sequences that are transcription binding domains and are recognized by the nucleic acid-binding domains of transcriptional proteins and/or transcription factors, repressors or enhancers etc. Typical regulatory sequences include, but are not limited to, transcriptional promoters, inducible promoters and transcriptional elements, an optional operate sequence to control transcription, a sequence encoding suitable mRNA ribosomal binding sites, and sequences to control the termination of transcription and/or translation.

Regulatory sequences can be a single regulatory sequence or multiple regulatory sequences, or modified regulatory sequences or fragments thereof. Modified regulatory sequences are regulatory sequences where the nucleic acid sequence has been changed or modified by some means, for example, but not limited to, mutation, methylation etc.

The term “operatively linked” as used herein refers to the functional relationship of the nucleic acid sequences with regulatory sequences of nucleotides, such as promoters, enhancers, transcriptional and translational stop sites, and other signal sequences. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined.

The articles “a” and “an” are used herein to refer to one or to more than one (i.e., to at least one) of the grammatical object of the article. By way of example, “an element” means one element or more than one element.

Metabolic Regulators

The present invention relates to the use of metabolic regulators to modulate metabolic function, for example metabolic regulators that affect muscle mass, fat mass, angiogenesis, insulin sensitivity and glucose sensitivity and cardiovascular function. The metabolic regulators of the present invention are at least one selected from the group of MSP1 (SEQ ID NO:16), MSP2 (SEQ ED NO:17), MSP3 (SEQ ID NO:1 and SEQ ID NO:2), MSP4 (SEQ ID NO:18), MSP5 (SEQ ID NO:12) and Insulin-like protein 6 (Ins16) (SEQ ID NO:20), as described below in more detail.

In one embodiment, the metabolic regulator of the present invention is Muscle Secreted Protein 1 (MSP1), which can be identified as RefSeq ID: BC52844 or as the Rikin clone 2160028F08Rik (SEQ ID NO: 16). In some embodiments, MSP1 is the human homologue of MSP1 or a human cognate of MSP1, and in some embodiments, MSP1 is a rodent isoform of MSP1 or any mammalian isoform of MSP1, for example primate MSP1. Also encompassed by the term MSP1 are all variants and homologues of MSP1, and functional derivatives of MSP 1, for example mutant variants and/or alternative isoforms of MSP1, for example alternative spliced isoforms of MSP1, of fragments of MSP1 or recombinant forms of MSP1.

In another embodiment, the metabolic regulator of the present invention is Muscle Secreted Protein 2 (MSP2), which can be identified as RefSeq ID: AK009779 or as the Rikin clone 2310043I08Rik (SEQ ID NO: 17). In some embodiments, MSP2 is the human homologue of MSP2 or a human cognate of MSP2, and in some embodiments, MSP2 is a rodent isoform of MSP2 or any mammalian isoform of MSP2, for example primate MSP2. Also encompassed by the term MSP2 are all variants and homologues of MSP2, and functional derivatives of MSP2, for example mutant variants and/or alternative isoforms of MSP2, for example alternative spliced isoforms of MSP2, of fragments of MSP2 or recombinant forms of MSP2.

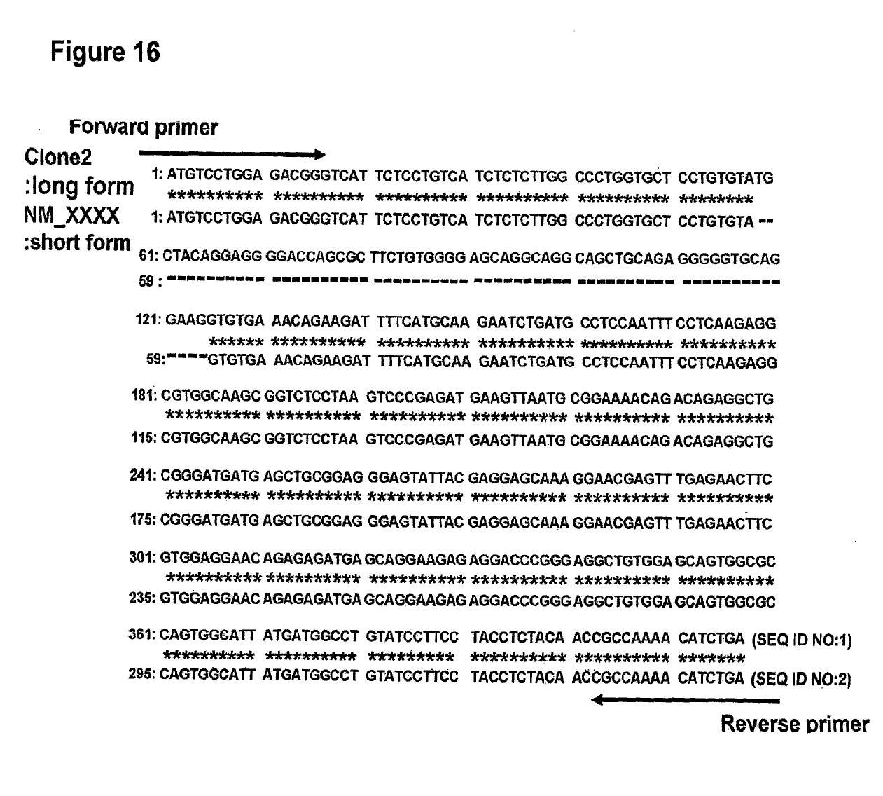

In another embodiment, the metabolic regulator of the present invention is Muscle Secreted Protein 2 (MSP3), which is identified as RefSeq ID: NM_{—}026754 or as the Rikin clone 1110017I16Rik (SEQ ID NO: 1) or the amino acid sequence NP_{—}660357 (SEQ ID NO:5). In some embodiments, MSP3 is the human homologue of MSP3 or a human cognate of MSP3, and in some embodiments, MSP3 is a rodent isoform of MSP3, for example amino acid sequence Refseq ID: NP_{—}081030 (SEQ ID NO:3) or XP_{—}001066258 (SEQ ID NO:4) or any mammalian isoform of MSP3, for example primate MSP3. Also encompassed by the term MSP3 are all variants and homologues of MSP3, and functional derivatives of MSP3, for example mutant variants and/or alternative isoforms of MSP3, for example alternative spliced isoforms of MSP3, for example the long isoform of MSP3 (SEQ ID NO: 1) or the short isoform of MSP3 (SEQ ID NO:2) as shown in FIG. 16, or fragments of MSP3 such as, for example MSP3 lacking the signal peptide, or recombinant forms of MSP3.

In another embodiment, the metabolic regulator of the present invention is Muscle Secreted Protein 4 (MSP4), which can be identified as RefSeq ID: BC025880 or Assesion NO: AK028883 (SEQ ID NO: 18) or as the Rikin clone 4732466D17Rik (SEQ ID NO: 18). In some embodiments, MSP4 is the human homologue of MSP4 or a human cognate of MSP4, and in some embodiments, MSP4 is a rodent isoform of MSP4 or any mammalian isoform of MSP4, for example primate MSP4. Also encompassed by the term MSP4 are all variants and homologues of MSP4, and functional derivatives of MSP4, for example mutant variants and/or alternative isoforms of MSP4, for example alternative spliced isoforms of MSP4, of fragments of MSP4 or recombinant forms of MSP4.

In another embodiment, the metabolic regulator of the present invention is Muscle Secreted Protein 5 (MSP5), which is identified as RefSeq ID: AK005465 or NM_{—}024237 or as the Rikin clone 1600015H20Rik (SEQ ID NO: 12) or the amino acid sequence NP_{—}694946 (SEQ ID NO: 15). In some embodiments, MSP5 is the human homologue of MSP5 or a human cognate of MSP5, and in some embodiments, MSP5 is a rodent isoform of MSP5, for example amino acid sequence Refseq ID: XP_{—}001081124(SEQ ID NO:13) or NP_{—}077199(SEQ ID NO:14) or any mammalian or non-mammalian animal isoform of MSP5, for example invertebrate MSP5 or primate MSP5. Also encompassed by the term MSP5 are all variants and homologues of MSP5, and functional derivatives of MSP5, for example mutant variants and/or alternative isoforms of MSP5, for example alternative spliced isoforms of MSP5, or fragments of MSP5 such as, for example MSP5 lacking the signal peptide, or recombinant forms of MSP5.

In another embodiment, the metabolic regulator of the present invention is Insulin-like protein 6 (Ins16), which is a member of the relaxin family as is identified as RefSeq ID: NM_{—}007179 (SEQ ID NO:20) or as Assession NO: AF_{—}156094 (SEQ ID NO: 20) or the amino acid sequence NP_{—}009110 (SEQ ID NO: 15). In some embodiments, Ins16 is the human homologue of Ins16 or a human cognate of Ins16, and in some embodiments, Ins16 is a rodent isoform of Ins16, or any mammalian or non-mammalian animal isoform of Ins16, for example invertebrate Ins16 or primate MSP5. Also encompassed by the term Ins16 are all variants and homologues of Ins16, and functional derivatives of Ins16, for example mutant variants and/or alternative isoforms of Ins16, for example alternative spliced isoforms of Ins16, or fragments of Ins16 such as, for example MSP5 lacking the signal peptide, or recombinant forms of Ins16.

One aspect of the present invention relates to compositions comprising agents that activate the metabolic regulators of the present invention. In some embodiment the agent is a nucleic acid sequence encoding a metabolic regulator of the present invention, for example the agent is a nucleic acid of an exogenous or endogenous gene encoding, for example MSP1, MSP2, MSP3, MPS4 MSP5 or Ins16, or variants or homologues or fragments or functional derivatives thereof. In some embodiments, the agent activates the expression of an endogenous metabolic regulator, for example MSP1, MSP2, MSP3, MPS4 MSP5 or Ins16. In another embodiment, the agent is a peptide or polypeptide of the metabolic regulator, for example MSP1, MSP2, MSP3, MPS4 MSP5 or Ins16 or a functional derivative or homologue or variant thereof, or a recombinant variant of a metabolic regulator. In alternative embodiments, an agent is an agonist of the metabolic regulator, for example an agent that induces the expression of the metabolic regulator or an agent that activates a peptide or gene product of metabolic regulator, for example but not limited to an agent that activates an inactive metabolic regulator protein or activates a metabolic regulator protein precursor or an agent that activates a post-transcriptional product or a metabolic regulator gene, for example activates a transcript, for example a mRNA transcript encoding a metabolic regulator. In some embodiments, where the agent is a nucleic acid encoding a metabolic regulator, the nucleic acid can encode a functional derivative or a variant or a recombinant version of a metabolic regulator.

In some embodiments, the nucleic acid encoding a metabolic regulator is a portion of genomic DNA, cDNA, mRNA, RNA or a fragment thereof encoding a functional metabolic regulator, for example MSP1, MSP2, MSP3, MPS4 MSP5 or Ins16 (e.g., a full length protein) or a functionally active fragment of, for example, MSP1, MSP2, MSP3, MPS4 MSP5 or Ins16 protein. The term “functionally active” refers to a fragment, derivative or analog having one or more functions associated with a full-length (wild-type) MSP1, MSP2, MSP3, MPS4 MSP5 or Ins16 polypeptide.

In some embodiments, where the agent is a nucleic acid encoding a metabolic regulator, the metabolic regulator is from the same species as the subject being administered the agent encoding a metabolic regulator, for example the metabolic regulator is human and the subject is human. In alternative embodiments, the nucleic acid encoding a metabolic regulator is from a different species, for example the nucleic acid encodes a metabolic regulator from a non-human mammal, for example a primate or mouse, and the subject is a human, or another example is the nucleic acid encodes a human metabolic regulator and the subject is a non-human animal, for example a transgenic animal, or rodent or a non-mammalian animal, for example a invertebrate, for example a zebrafish, and in some embodiments the subject is an animal is a transgenic animal (e.g., a mouse metabolic regulators overexpressed in a mouse). In some embodiments, the metabolic regulator is a cognate heterologous gene of MSP1, MSP2, MSP3, MPS4 MSP5 or Ins16. A cognate heterologous MSP1, MSP2, MSP3, MPS4 MSP5 or Ins16 gene refers to a corresponding gene from another species; thus, if murine MSP1, MSP2, MSP3, MPS4 MSP5 or Ins16 is the reference, human MSP1, MSP2, MSP3, MPS4 MSP5 or Ins16 gene is a cognate heterologous gene (as is porcine, ovine, or rat muscle related, along with muscle related genes from other species). In some embodiments, the MSP1, MSP2, MSP3, MPS4 MSP5 or Ins16 gene can encode a human MSP1, MSP2, MSP3, MPS4 MSP5 or Ins16 or a functionally active fragment thereof.

The metabolic regulators, for example MSP1, MSP2, MSP3, MPS4 MSP5 or Ins16 protein can be a “homologous” or “heterologous polypeptides.” A “heterologous polypeptide,” also referred to as a “xenogenic polypeptide,” is a polypeptide having an amino acid sequence found in an organism not consisting of the transgenic non-human animal. As used herein, the term “polypeptide” refers to a polymer of amino acids and its equivalent and does not refer to a specific length of the product; thus, peptides, oligopeptides and proteins are included within the definition of a polypeptide. A derivative is a polypeptide having conservative amino acid substitutions, as compared with another sequence. Derivatives further include other modifications of proteins, including, for example, modifications such as glycosylations, acetylations, phosphorylations, and the like.

The metabolic regulators, for example MSP1, MSP2, MSP3, MPS4 MSP5 or Ins16 genes containing various gene segments encoding a cognate heterologous protein sequence may be readily identified, e.g. by hybridization or DNA sequencing, as being from a species of organism other than the transgenic animal. In some embodiments, the cognate metabolic regulatorsis at least 75%, at least 80%, at least 85%, at least 90% or at least 95% identical to the homologous muscle related transgene. As used herein, the terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence, as measured using one of the following sequence comparison algorithms, or by visual inspection. The phrase “substantially identical,” in the context of two nucleic acids or polypeptides, refers to two or more sequences or subsequences that have at least 60%, typically 80%, most typically 90-95% nucleotide or amino acid residue identity, when compared and aligned for maximum correspondence, as measured using one of the following sequence comparison algorithms, or by visual inspection. An indication that two polypeptide sequences are “substantially identical” is that one polypeptide is immunologically reactive with antibodies raised against the second polypeptide.

In some embodiments, the metabolic regulators, for example MSP1, MSP2, MSP3, MPS4 MSP5 or Ins16 protein is at least 75%, at least 80%, at least 85%, at least 90% or at least 95% similar to the homologous MSP1, MSP2, MSP3, MPS4 MSP5 or Ins16 protein. As used herein, “similarity” or “percent similarity” in the context of two or polypeptide sequences, refer to two or more sequences or subsequences that are the same or have a specified percentage of amino acid residues or conservative substitutions thereof, that are the same, when compared and aligned for maximum correspondence, as measured using one of the following sequence comparison algorithms, or by visual inspection. By way of example, a first amino acid sequence can be considered similar to a second amino acid sequence when the first amino acid sequence is at least 50%, 60%, 70%, 75%, 80%, 90%, or even 95% identical, or conservatively substituted, to the second amino acid sequence when compared to an equal number of amino acids as the number contained in the first sequence, or when compared to an alignment of polypeptides that has been aligned by a computer similarity program known in the art, as discussed below.

The term “substantial similarity” in the context of polypeptide sequences, indicates that the polypeptide comprises a sequence with at least 60% sequence identity to a reference sequence, or 70%, or 80%, or 85% sequence identity to the reference sequence, or most preferably 90% identity over a comparison window of about 10-20 amino acid residues. In the context of amino acid sequences, “substantial similarity” further includes conservative substitutions of amino acids. Thus, a polypeptide is substantially similar to a second polypeptide, for example, where the two peptides differ by one or more conservative substitutions.

Determination of the human homologs of the genes of the present invention may be easily ascertained by the skilled artisan. “Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present application.

In one embodiment, the term “human homolog” to a gene transcript identified as associated with metabolic regulator refers to a DNA sequence that has at least about 55% homology to the full length nucleotide sequence of the sequence of the MSP1, MSP2, MSP3, MPS4 MSP5 or Ins16 gene as encoded by the genome of humans or an animal, for example mouse or transgenic animal. In one embodiment, the term “human homolog” to a protein identified as associated with MSP1, MSP2, MSP3, MPS4 MSP5 or Ins16 refers to an amino acid sequence that has 40% homology to the full length amino acid sequence of the protein identified as associated with MSP1, MSP2, MSP3, MPS4 MSP5 or Ins16 as encoded by the genome of the transgenic animal of the present invention, more preferably at least about 50%, still more preferably, at least about 60% homology, still more preferably, at least about 70% homology, even more preferably, at least about 75% homology, yet more preferably, at least about 80% homology, even more preferably at least about 85% homology, still more preferably, at least about 90% homology, and more preferably, at least about 95% homology. As discussed above, the homology is at least about 50% to 100% and all intervals in between (i.e., 55%, 60%, 70%, 75%, 80%, 85%, 90%, 95%, 98%, etc.).

The term “conservative substitution,” when describing a polypeptide, refers to a change in the amino acid composition of the polypeptide that does not substantially alter the polypeptide's activity. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not substantially alter activity. Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984).) In addition, individual substitutions, deletions or additions that alter, add or delete a single amino acid or a small percentage of amino acids in an encoded sequence are also “conservative substitutions.”

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith and Waterman (Adv. Appl. Math. 2:482 (1981), which is incorporated by reference herein), by the homology alignment algorithm of Needleman and Wunsch (J. Mol. Biol. 48:443-53 (1970), which is incorporated by reference herein), by the search for similarity method of Pearson and Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4th ed., John Wiley and Sons, New York (1999)).

One example of a useful algorithm is PILEUP. PILEUP creates a multiple sequence alignment from a group of related sequences using progressive, pairwise alignments to show the percent sequence identity. It also plots a tree or dendogram showing the clustering relationships used to create the alignment. PILEUP uses a simplification of the progressive alignment method of Feng and Doolittle (J. Mol. Evol. 25:351-60 (1987), which is incorporated by reference herein). The method used is similar to the method described by Higgins and Sharp (Comput. Appl. Biosci. 5:151-53 (1989), which is incorporated by reference herein). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described by Altschul et al. (J. Mol. Biol. 215:403-410 (1990), which is incorporated by reference herein). (See also Zhang et al., Nucleic Acid Res. 26:3986-90 (1998); Altschul et al., Nucleic Acid Res. 25:3389-402 (1997), which are incorporated by reference herein). Software for performing BLAST analyses is publicly available through the National Center for Biotechnology Information internet web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff and Henikoff, Proc. Natl. Acad. Sci. USA 89:10915-9 (1992), which is incorporated by reference herein) alignments (B) of 50, expectation (E) of 10, M=5, N=4, and a comparison of both strands.

In addition to calculating percent sequence identity, the BLAST algorithm also performs a statistical analysis of the similarity between two sequences (see, e.g., Karlin and Altschul, Proc. Natl. Acad. Sci. USA 90:5873-77 (1993), which is incorporated by reference herein). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more typically less than about 0.01, and most typically less than about 0.001.

Homologues of the metabolic regulators of the present invention, for example MSP1, MSP2, MSP3, MPS4 MSP5 or Ins16 are also encompassed for use in the present invention, and can also be identified, for example, by expression of MSP1, MSP2, MSP3, MPS4 MSP5 or Ins16 from an expression library. (See, e.g., Sambrook et al. (2001). Molecular cloning: a laboratory manual, 3rd ed. (Cold Spring Harbor, N.Y., Cold Spring Harbor Laboratory Press); Ausubel et al., supra.) A mutated endogenous gene sequence can be referred to as a heterologous transgene; for example, a transgene encoding a mutation in a murine muscle related gene which is not known in naturally-occurring murine genomes is a heterologous transgene with respect to murine and non-murine species. The MSP1, MSP2, MSP3, MPS4 MSP5 or Ins16 genes also can encode a modified MSP1, MSP2, MSP3, MPS4 MSP5 or Ins16, protein, such as, for example, those disclosed in U.S. Patent Publication Nos. 2004/0048255 and 2004/0132156 (the disclosures of which are incorporated by reference herein).

### Agents

Agents useful in the present invention are any entity, biological or chemical that targets the activity of a metabolic regulator of the present invention. An agent can function as an agonist to activate the metabolic regulator or an antagonist to suppress or inhibit the activity of the metabolic regulator. An agent can be a small molecule, protein, polypeptide, nucleic acid, antisense nucleic acids, RNAi, such as siRNA or shRNA, peptides, peptidomimetics, receptors, ligands, and antibodies, aptamers, polypeptides, nucleic acid analogues or variants thereof, including an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

An agent may operate to prevent transcription, translation, post-transcriptional or post-translational processing or otherwise to affect the activity of the protein, polypeptide or polynucleotide in any way, via either direct of indirect action. An agent may for example be a nucleic acid, peptide, or any other suitable chemical compound or molecule or any combination of these. Additionally, it will be understood that an agent can indirectly affect the activity of a protein, polypeptide of polynucleotide, for example but not limited to, the agent may affect the activity of the cellular molecules which may in turn act as regulators or the protein, polypeptide or polynucleotide itself. Similarly, the agent may affect the activity of molecules which are themselves subject to the regulation or modulation by the protein, polypeptide of polynucleotide.

In some embodiments, an agent acts at the level of gene transcription to modulate gene expression, for example an agonist would activate gene expression and an antagonist to inhibit gene transcription. In some embodiments, an agent acts at the level of protein to modulate protein function, for example an agonist will activate and/or enhance activity of the protein, whereas an antagonist can inhibit and/or suppress the activity of the protein. In an alternative embodiment, an agent is a nucleic acid or nucleic acid analogue encoding a metabolic regulator. In another alternative embodiment, an agent is a peptide or polypeptide, for example an agonist is a biological active or functional derivative of a protein, whereas an antagonist can be, for example, a inhibitory polypeptide or dominant negative polypeptide.

Polypeptides of metabolic regulators of the present invention, fragments and derivatives thereof can be obtained by any suitable method. For example, polypeptides can be produced using conventional recombinant nucleic acid technology such as DNA or RNA, preferably DNA. Guidance and information concerning methods and materials for production of polypeptides using recombinant DNA technology can be found in numerous treatises and reference manuals. See, e.g., Sambrook et al, 1989, Molecular Cloning—A Laboratory Manual, 2nd Ed., Cold Spring Harbor Press; Ausubel et al. (eds.), 1994, Current Protocols in Molecular Biology, John Wiley & Sons, Inc.; Innis et al. (eds.), 1990 PCR Protocols, Academic Press.

Alternatively, polypeptides of metabolic regulators of the present invention or fragments thereof can be obtained directly by chemical synthesis, e.g., using a commercial peptide synthesizer according to vendor's instructions. Methods and materials for chemical synthesis of polypeptides are well known in the art. See, e.g., Merrifield, 1963, “Solid Phase Synthesis,” J. Am. Chem. Soc. 83:2149-2154.

A polypeptide of a metabolic regulators of the present invention, can be introduced into a cell using conventional techniques for transporting proteins into intact cells, e.g., by fusing the polypeptide to the internalization peptide sequence derived from Antennapedia (Bonfanti et al., Cancer Res. 57:1442-1446) or to a nuclear localization protein such as HIV tat peptide (U.S. Pat. No. 5,652,122).

Alternatively, a polypeptide of metabolic regulators of the present invention, can be expressed in the cell following introduction of a DNA encoding the protein, e.g., a nucleic acid encoding a metabolic regulator, for example MSP2, MSP3, MSP4, MPS5 and Ins16 or homologues or functional derivatives thereof, e.g., in a conventional expression vector or by a catheter or by ex vivo transplants.

In some embodiments, agents that are polypeptides or peptides of the metabolic regulators of the present invention, for example MSP2, MSP3, MSP4, MPS5 and Ins16 or fragments or homologues or variants thereof are cleavable peptides. Cleavable peptide is a peptide comprising an amino acid sequence that is recognized by a protease or peptidase or other cleaving agent expressed by a cell and found in surrounding tissue, or produced by a microbe capable of establishing an infection in a mammal. Enzyme-cleavable peptides can, but are not required to, contain one or more amino acids in addition to the amino acid recognition sequence; additional amino acids can be added to the amino terminal, carboxy terminal, or both the amino and carboxy terminal ends of the recognition sequence. Means of adding amino, acids to an amino acid sequence, e.g., in an automated peptide synthesizer, as well as means of detecting cleavage of a peptide, e.g., by chromatographic analysis for the amino acid products of such cleavage, are well known to ordinarily skilled artisans given the teachings of this invention. Enzyme-cleavable peptides, typically from about 2 to 20 amino acids in length.

The peptide or polypeptide agent useful in the present invention can be modified at their amino termini, for example, so as to increase their hydrophilicity. Increased hydrophobicity enhances exposure of the peptides on the surfaces of lipid-based carriers into which the parent peptide-lipid conjugates have been incorporated. Polar groups suitable for attachment to peptides so as to increase their hydrophilicity are well known, and include, for example and without limitation: acetyl (“Ac”), 3-cyclohexylalanyl (“Cha”), acetyl-serine (“Ac Ser”), acetyl-seryl-serine (“Ac-Ser-Ser-”), succinyl (“Suc”), succinyl-serine (“Suc-Ser”), succinyl-seryl-serine (“Suc-Ser-Ser”), methoxy succinyl (“MeO-Suc”), methoxy succinyl-serine (“MeO-Suc-Ser”), methoxy succinyl-seryl-serine (“MeO-Suc-Ser-Ser”) and seryl-serine (“Ser-Ser-”) groups, polyethylene glycol (“PEG”), polyacrylamide, polyacrylomorpholine, polyvinylpyrrolidine, a polyhydroxyl group and carboxy sugars, e.g., lactobionic, N-acetyl neuraminic and sialic acids, groups. The carboxy groups of these sugars would be linked to the N-terminus of the peptide via an amide linkage. Presently, the preferred N-terminal modification is a methoxy-succinyl modification.

### Gene Therapy

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contain nucleic acid sequences encoding the human homologs of metabolic regulators of the present invention are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding the human homolog of the factor associated with muscle growth to be used in gene therapy are cloned into one or more vectors, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Cin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Another preferred viral vector is a pox virus such as a vaccinia, for example an attenuated vaccinia such as Modified Virus Ankara (MVA) or NYVAC, an avipox such as fowl pox or canary pox. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In one preferred embodiment, adenovirus vectors are used. In another embodiment, lentiviral vectors are used, such as the HIV based vectors described in U.S. Pat. Nos. 6,143,520; 5,665,557; and 5,981,276, which are herein incorporated by reference.

Use of Adeno-associated virus (AAV) vectors are also contemplated (Walsh et al., Proc. Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

U.S. Pat. No. 5,676,954 (which is herein incorporated by reference) reports on the injection of genetic material, complexed with cationic liposomes carriers, into mice. U.S. Pat. Nos. 4,897,355, 4,946,787, 5,049,386, 5,459,127, 5,589,466, 5,693,622, 5,580,859, 5,703,055, and international publication NO: WO 94/9469 (which are herein incorporated by reference) provide cationic lipids for use in transfecting DNA into cells and mammals. U.S. Pat. Nos. 5,589,466, 5,693,622, 5,580,859, 5,703,055, and international publication NO: WO 94/9469 (which are herein incorporated by reference) provide methods for delivering DNA-cationic lipid complexes to mammals. Such cationic lipid complexes or nanoparticles can also be used to deliver protein. The protein will preferably contain a nuclear localization sequence.

For general reviews of the methods of gene and protein therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

A gene or nucleic acid sequence can be introduced into a target cell by any suitable method. For example, a metabolic regulators can be introduced into a cell by transfection (e.g., calcium phosphate or DEAE-dextran mediated transfection), lipofection, electroporation, microinjection (e.g., by direct injection of naked DNA), biolistics, infection with a viral vector containing a muscle related transgene, cell fusion, chromosome-mediated gene transfer, microcell-mediated gene transfer, nuclear transfer, and the like. A nucleic acid agent of encoding a metabolic regulator can be introduced into cells by electroporation (see, e.g., Wong and Neumann, Biochem. Biophys. Res. Commun. 107:584-87 (1982)) and biolistics (e.g., a gene gun; Johnston and Tang, Methods Cell Biol 43 Pt A:353-65 (1994); Fynan et al., Proc. Natl. Acad. Sci. USA 90:11478-82 (1993)).

In certain embodiments, a gene or nucleic acid sequence encoding the metabolic regulator, for example MSP1, MPS2, MSP3, MSP4, MSP5 or Ins16 or functional derivatives thereof can be introduced into target cells by transfection or lipofection. Suitable agents for transfection or lipofection include, for example, calcium phosphate, DEAE dextran, lipofectin, lipfectamine, DIMRIE C, Superfect, and Effectin (Qiagen), unifectin, maxifectin, DOTMA, DOGS (Transfectam; dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecyl-N,N-dihydroxyethylammonium bromide), polybrene, poly(ethylenimine) (PEI), and the like. (See, e.g., Banerjee et al., Med. Chem. 42:4292-99 (1999); Godbey et al., Gene Ther. 6:1380-88 (1999); Kichler et al., Gene Ther. 5:855-60 (1998); Birchaa et al., S. Pharm. 183:195-207 (1999); each

A gene or nucleic acid sequence encoding metabolic regulator, for example MSP1, MPS2, MSP3, MSP4, MSP5 or Ins16, or functional derivatives thereof can also be introduced into cells by infection of cells or into cells of a zygote with an infectious virus containing the mutant gene. Suitable viruses include retroviruses (see generally Jaenisch, Proc. Natl. Acad. Sci. USA 73:1260-64 (1976)); defective or attenuated retroviral vectors (see, e.g., U.S. Pat. No. 4,980,286; Miller et al., Meth. Enzymol. 217:581-99 (1993); Boesen et al., Biotherapy 6:291-302 (1994); these references are incorporated herein in their entirety), lentiviral vectors (see, e.g., Naldini et al., Science 272:263-67 (1996), incorporated by reference herein in its entirety), adenoviruses or adeno-associated virus (AAV) (see, e.g., Ali et al., Gene Therapy 1:367-84 (1994); U.S. Pat. Nos. 4,797,368 and 5,139,941; Walsh et al., Proc. Soc. Exp. Biol. Med. 204:289-300 (1993); Grimm et al., Human Gene Therapy 10:2445-50 (1999); the disclosures of which are incorporated by reference herein in their entirety).

Viral vectors can be introduced into, for example, embryonic stem cells, primordial germ cells, oocytes, eggs, spermatocytes, sperm cells, fertilized eggs, zygotes, blastomeres, or any other suitable target cell. In an exemplary embodiment, retroviral vectors which transduce dividing cells (e.g., vectors derived from murine leukemia virus; see, e.g., Miller and Baltimore, Mol. Cell. Biol. 6:2895 (1986)) can be used. The production of a recombinant retroviral vector carrying a gene of interest is typically achieved in two stages. First, a metabolic regulators can be inserted into a retroviral vector which contains the sequences necessary for the efficient expression of the metabolic regulators (including promoter and/or enhancer elements which can be provided by the viral long terminal repeats (LTRs) or by an internal promoter/enhancer and relevant splicing signals), sequences required for the efficient packaging of the viral RNA into infectious virions (e.g., a packaging signal (Psi), a tRNA primer binding site (−PBS), a 3-[prime] regulatory sequence required for reverse transcription (+PBS)), and a viral LTRs). The LTRs contain sequences required for the association of viral genomic RNA, reverse transcriptase and integrase functions, and sequences involved in directing the expression of the genomic RNA to be packaged in viral particles.

Following the construction of the recombinant vector, the vector DNA is introduced into a packaging cell line. Packaging cell lines provide viral proteins required in trans for the packaging of viral genomic RNA into viral particles having the desired host range (e.g., the viral-encoded core (gag), polymerase (pol) and envelope (env) proteins). The host range is controlled, in part, by the type of envelope gene product expressed on the surface of the viral particle. Packaging cell lines can express ecotrophic, amphotropic or xenotropic envelope gene products. Alternatively, the packaging cell line can lack sequences encoding a viral envelope (env) protein. In this case, the packaging cell line can package the viral genome into particles which lack a membrane-associated protein (e.g., an env protein). To produce viral particles containing a membrane-associated protein which permits entry of the virus into a cell, the packaging cell line containing the retroviral sequences can be transfected with sequences encoding a membrane-associated protein (e.g., the G protein of vesicular stomatitis virus (VSV)). The transfected packaging cell can then produce viral particles which contain the membrane-associated protein expressed by the transfected packaging cell line; these viral particles which contain viral genomic RNA derived from one virus encapsidated by the envelope proteins of another virus are said to be pseudotyped virus particles.

Methods known in the art for the therapeutic delivery of agents such as proteins and/or nucleic acids can be used for the delivery of an agent polypeptide or an agent nucleic acid encoding a metabolic regulator of the present invention for modulating a metabolic function in a subject, e.g., cellular transfection, gene therapy, direct administration with a delivery vehicle or pharmaceutically acceptable carrier, indirect delivery by providing recombinant cells comprising a nucleic acid encoding a targeting fusion polypeptide of the invention.

Various delivery systems are known and can be used to administer the agent polypeptide of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, pulmonary, intranasal, intraocular, epidural, and oral routes. The agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g.; by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, the implant being of a porous, non-porous, or gelatinous material, including membranes, such as sialastic membranes, fibers, or commercial skin substitutes.

In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105). In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

Antibodies. In some embodiments, agent of the metabolic regulator of the present invention, for example MSP1, MPS2, MPS3, MPS4, MPS5 and Ins16 include, for example, antibodies, including monoclonal, chimeric humanized and recombinant antibodies and fragment thereof. In some embodiments, neutralizing antibodies can be used as agents that are antagonists of the metabolic regulators of the present invention, for example inhibitors of MSP1, MPS2, MPS3, MPS4, MPS5 and Ins16 or their homologues. Antibodies are readily raised in animals such as rabbits or mice by immunization with the gene product, which when inactivated, potentiate the effect of an antimicrobial agent. Immunized mice are particularly useful for providing sources of B cells for the manufacture of hybridomas, which in turn are cultured to produce large quantities of monoclonal antibodies. Chimeric antibodies are immunoglobin molecules characterized by two or more segments or portions derived from different animal species. Generally, the variable region of the chimeric antibody is derived from a non-human mammalian antibody, such as murine monoclonal antibody, and the immunoglobin constant region is derived from a human immunoglobin molecule. Preferably, both regions and the combination have low immunogenicity as routinely determined. Humanized antibodies are immunoglobin molecules created by genetic engineering techniques in which the murine constant regions are replaced with human counterparts while retaining the murine antigen binding regions. The resulting mouse-human chimeric antibody should have reduced immunogenicity and improved pharmacokinetics in humans. Some examples of high affinity monoclonal antibodies and chimeric derivatives thereof, useful in the methods of the present invention, are described in the European Patent Application EP 186,833; PCT Patent Application WO 92/16553; and U.S. Pat. No. 6,090,923.

Antibodies provide high binding avidity and unique specificity to a wide range of target antigens and haptens. Monoclonal antibodies useful in the practice of the present invention include whole antibody and fragments thereof and are generated in accordance with conventional techniques, such as hybridoma synthesis, recombinant DNA techniques and protein synthesis. Useful monoclonal antibodies and fragments may be derived from any species (including humans) or may be formed as chimeric proteins which employ sequences from more than one species. Human monoclonal antibodies or “humanized” murine antibody are also used in accordance with the present invention. For example, murine monoclonal antibody may be “humanized” by genetically recombining the nucleotide sequence encoding the murine Fv region (i.e., containing the antigen binding sites) or the complementarily determining regions thereof with the nucleotide sequence encoding a human constant domain region and an Fc region. Humanized targeting moieties are recognized to decrease the immunoreactivity of the antibody or polypeptide in the host recipient, permitting an increase in the half-lfe and a reduction n the possibly of adverse immune reactions in a manner similar to that disclosed in European Patent Application No. 0,411,893 A2. The murine monoclonal antibodies should preferably be employed in humanized form. Antigen binding activity is determined by the sequences and conformation of the amino acids of the six complementarily determining regions (CDRs) that are located (three each) on the light and heavy chains of the variable portion (Fv) of the antibody. The 25-kDa single-chain Fv (scFv) molecule, composed of a variable region (VL) of the light chain and a variable region (VH) of the heavy chain joined via a short peptide spacer sequence, is the smallest antibody fragment developed to date. Techniques have been developed to display scFv molecules on the surface of filamentous phage that contain the gene for the scFv. scFv molecules with a broad range of antigenic-specificities can be present in a single large pool of scFv-phage library.

One limitation of scFv molecules is their monovalent interaction with target antigen. One of the easiest methods of improving the binding of a scFv to its target antigen is to increase its functional affinity through the creation of a multimer. Association of identical scFv molecules to form diabodies, triabodies and tetrabodies can comprise a number of identical Fv modules. These reagents are therefore multivalent, but monospecific. The association of two different scFv molecules, each comprising a VH and VL domain derived from different parent Ig will form a fully functional bispecific diabody. A unique application of bispecific scFvs is to bind two sites simultaneously on the same target molecule via two (adjacent) surface epitopes. These reagents gain a significant: avidity advantage over a single scFv or Fab fragments. A number of multivalent scFv-based structures has been engineered, including for example, miniantibodies, dimeric miniantibodies, minibodies, (scFv)2, diabodies and triabodies. These molecules span a range of valence (two to four binding sites), size (50 to 120 kDa), flexibility and ease of production. Single chain Fv antibody fragments (scFvs) are predominantly monomeric when the VH and VL domains are joined by, polypeptide linkers of at least 12 residues. The monomer scFv is thermodynamically stable with: linkers of 12 and 25 amino acids length under all conditions. The noncovalent diabody and triabody molecules are easy to engineer and are produced by shortening the peptide linker that connects the variable heavy and variable light chains of a single scFv molecule. The scFv dimers are joined by amphipathic helices that offer a high degree of flexibility and the miniantibody structure can be modified to create a dimeric bispecific (DiBi) miniantibody that contains two miniantibodies (four scFv molecules) connected via a double helix. Gene-fused or disulfide bonded scFv dimers provide an intermediate degree of flexibility and are generated by straightforward cloning techniques adding a C-terminal Gly4Cys sequence. scFv-CH3 minibodies are comprised of two scFv molecules joined to an IgG CH3 domain either directly (LD minibody) or via a very flexible hinge region (Flex minibody). With a molecular weight of approximately 80 kDa, these divalent constructs are capable of significant binding to antigens. The Flex minibody exhibits impressive tumor localization in mice. Bi- and tri-specific multimers can be formed by association of different scFv molecules. Increase in functional affinity can be reached when Fab or single chain Fv antibody fragments (scFv) fragments are complexed into dimers, trimers or larger aggregates. The most important advantage of multivalent scFvs over monovalent scFv and Fab fragments is the gain in functional binding affinity (avidity) to target antigens. High avidity requires that scFv multimers are capable of binding simultaneously to separate target antigens. The gain in functional affinity for scFv diabodies compared to scFv monomers is significant and is seen primarily in reduced off-rates, which result from multiple binding to two or more target antigens and to rebinding when one Fv dissociates. When such scFv molecules associate into multimers, they can be designed with either high avidity to a single target antigen or with multiple specificities to different target antigens. Multiple binding to antigens is dependent on correct alignment and orientation in the Fv modules. For full avidity in multivalent scFvs target, the antigen binding sites must point towards the same direction. If multiple binding is not sterically possible then apparent gains in functional affinity are likely to be due the effect of increased rebinding, which is dependent on diffusion rates and antigen concentration. Antibodies conjugated with moieties that improve their properties are also contemplated for the instant invention. For example, antibody conjugates with PEG that increases their half-life in vivo can be used for the present invention. Immune libraries are prepared by subjecting the genes encoding variable antibody fragments from the B lymphocytes of naive or immunized animals or patients to PCR amplification. Combinations of oligonucleotides which are specific for immunoglobulin genes or for the immunoglobulin gene families are used. Immunoglobulin germ line genes can be used to prepare semisynthetic antibody repertoires, with the complementarily-determining region of the variable fragments being amplified by PCR using degenerate primers. These single-pot libraries have the advantage that antibody fragments against a large number of antigens can be isolated from one single library. The phage-display technique can be used to increase the affinity of antibody fragments, with new libraries being prepared from already existing antibody fragments by random, codon-based or site-directed mutagenesis, by shuffling the chains of individual domains with those of fragments from naive repertoires or by using bacterial mutator strains.

Alternatively, a SCID-hu mouse, for example the model developed by Genpharm, can be used to produce antibodies, or fragments thereof. In one embodiment, a new type of high avidity binding molecule, termed peptabody, created by harnessing the effect of multivalent interaction is contemplated. A short peptide ligand was fused via a semirigid hinge region with the coiled-coil assembly domain of the cartilage oligomeric matrix protein, resulting in a pentameric multivalent binding molecule. In preferred embodiment of this invention, ligands and/or chimeric inhibitors can be targeted to tissue- or tumor-specific targets by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. Alternatively, two or more active agents and or inhibitors attached to targeting moieties can be administered, wherein each conjugate includes a targeting moiety, for example, a different antibody. Each antibody is reactive with a different target site epitope (associated with the same or a different target site antigen). The different antibodies with the agents attached accumulate additively at the desired target site. Antibody-based or non-antibody-based targeting moieties may be employed to deliver an agent to a target site as discussed in more detail below. Preferably, a natural binding agent for an unregulated antigen is used for this purpose.

Agents that are antibodies can be agonists and/or antagonists, for example, an antibody that functions as an agonist increases the activity of the polypeptide and/or gene expression, whereas an antibody that functions as an antagonist reduces the activity of the polypeptide and/or gene expression. An example of such an antibody that functions as an antagonist is, for example but not limited to a neutralizing antibody.

It will be appreciated by those of skill that the metabolic regulators of the present invention, for example MSP1, MPS2, MPS3, MPS4, MPS5 and Ins16 or their homologues can be readily manipulated to alter the amino acid sequence of a protein. Genes encoding the metabolic regulators of the present invention, for example MSP1, MPS2, MPS3, MPS4, MPS5 and Ins16 or their homologues can be manipulated by a variety of well known techniques for in vitro mutagenesis, among others, to produce variants of the naturally occurring human protein or fragment thereof, herein referred to as variants or muteins, may be used in accordance with the invention.

The variation in primary structure of variants of the metabolic regulators of the present invention, for example MSP1, MPS2, MPS3, MPS4, MPS5 and Ins16 or their homologues are useful in the invention, for instance, may include deletions, additions and substitutions. The substitutions may be conservative or non-conservative. The differences between the natural protein (or wild-type protein) and the variant generally conserve desired properties, mitigate or eliminate undesired properties and add desired or new properties. For example, variants of metabolic regulators may have superior activity as compared to wild-type metabolic regulators and this function as agonists, whereas variants with decrease activity or inhibit or have the opposite function as compared to the wild type metabolic regulator function as an antagonist, for example, but not limited to a dominant negative form of a metabolic regulator.

Antagonists agents. Similarly, techniques for making small oligopeptides and polypeptides that inactivate and/or function as dominant negative versions (i.e. inactive versions) of larger proteins from which they are derived are well known and have become routine in the art. Thus, peptide analogs of gene products of the invention that inactivate or inhibit the metabolic regulators of the present invention, for example MSP1, MPS2, MPS3, MPS4, MPS5 and Ins16 or their homologues also are useful in the invention.

In some embodiments, RNA interference or “RNAi” can be used as agents that function as antagonists of the metabolic regulators of the present invention, for example MSP1, MPS2, MPS3, MPS4, MPS5 and Ins16 or their homologues. In such an embodiment, a RNAi molecule that negatively regulates the expression of the metabolic regulators of the present invention, for example MSP1, MPS2, MPS3, MPS4, MPS5 and Ins16 or their homologues of the invention, can be used as to modulate metabolic function. RNAi is a term initially coined by Fire and co-workers to describe the observation that double-stranded RNA (dsRNA) can block gene expression when it is introduced into worms (Fire et al. (1998) Nature 391, 806-811). dsRNA directs gene-specific, post-transcriptional silencing in many organisms, including vertebrates, and has provided a new tool for studying gene function. RNAi involves mRNA degradation of a target gene. Results showed that RNAi is ATP-dependent yet uncoupled from mRNA translation. That is, protein synthesis is not required for RNAi in vitro. In the RNAi reaction, both strands (sense and antisense) of the dsRNA are processed to small RNA fragments or segments of from about 21 to about 23 nucleotides (nt) in length (RNAs with mobility in sequencing gels that correspond to markers that are 21-23 nt in length, optionally referred to as 21-23 nt RNA). Processing of the dsRNA to the small RNA fragments does not require the targeted mRNA, which demonstrates that the small RNA species is generated by processing of the dsRNA and not as a product of dsRNA-targeted mRNA degradation. The mRNA is cleaved only within the region of identity with the dsRNA. Cleavage occurs at sites 21-23 nucleotides apart, the same interval observed for the dsRNA itself, suggesting that the 21-23 nucleotide fragments from the dsRNA are guiding mRNA cleavage. Isolated RNA molecules (double-stranded; single-stranded) of from about 21 to about 23 nucleotides mediate RNAi. That is, the isolated RNAs mediate degradation of mRNA of a gene to which the mRNA corresponds (mediate degradation of mRNA that is the transcriptional product of the gene, which is also referred to as a target gene). Isolated RNA molecules specific to G6PD mRNA, which mediate RNAi, are antagonists useful in the method of the present invention. Alternative nucleic acid and nucleic acid analogues can be used as enhancers of antimicrobial peptides, for example oligonucleotides, antisense nucleic acid constructs, siRNA, microRNA, shRNA etc.

In some embodiments of the invention agents that function as antagonists of the metabolic regulators of the present invention, for example MSP1, MPS2, MPS3, MPS4, MPS5 and Ins16 or their homologues may be administered to the subject in a vector. The vector may be a plasmid vector, a viral vector, or any other suitable vehicle adapted for the insertion and foreign sequence and for the introduction into eukaryotic cells. The vector can be an expression vector capable of directing the transcription of the DNA sequence of the agonist or antagonist nucleic acid molecules into RNA. Viral expression vectors can be selected from a group comprising, for example, reteroviruses, lentiviruses, Epstein Barr virus-, bovine papilloma virus, adenovirus- and adeno-associated-based vectors or hybrid virus of any of the above. In one embodiment, the vector is episomal. The use of a suitable episomal vector provides a means of maintaining the agonist or antagonist nucleic acid molecule in the subject in high copy number extra chromosomal DNA thereby eliminating potential effects of chromosomal integration.

Another embodiment of the invention, agents that function as antagonists of the metabolic regulators of the present invention, for example MSP1, MPS2, MPS3, MPS4, MPS5 and Ins16 or their homologues may be achieved by introducing catalytic antisense nucleic acid constructs, such as ribozymes, which are capable of cleaving RNA transcripts and thereby preventing the production of wildtype protein. Ribozymes are targeted to and anneal with a particular sequence by virtue of two regions of sequence complementary to the target flanking the ribozyme catalytic site. After binding the ribozyme cleaves the target in a site specific manner. The design and testing of ribozymes which specifically recognize and cleave sequences of the metabolic regulators of the present invention, for example MSP1, MPS2, MPS3, MPS4, MPS5 and Ins16 or their homologues, can be achieved by techniques well known to those in the art (for example Lleber and Strauss, (1995) Mol Cell Biol 15:540.551, the disclosure of which is incorporated herein by reference).

Targeting. In some embodiments, the agent of the invention is targeted to muscle via a targeting ligand. A targeting ligand is a molecule, e.g., a protein or fragment thereof that specifically binds with high affinity to a target on a pre-selected cell, such as a surface protein such as a receptor that is present to a greater degree on the pre-selected cell target than on any other body tissue. For example, as described in U.S. Pat. Nos. 5,814,478 and 6,413,740, the MuSK receptor is highly specific to muscle. Accordingly, the cognate ligand agrin, as well as MuSK binding portions thereof is an example of a targeting ligand useful to target the agent to muscle. In some embodiments, the targeting ligand is fused to an agent of the present invention, for example fused to an polypeptide of a metabolic regulator of the present invention, for example MSP1, MPS2, MPS3, MSP4, MPS5 or Ins16, or homologues, variants or fragments thereof. In some embodiments, the targeting ligand can be fused to a nucleotide agent of the present invention. Another example of a targeting ligand is a group of cadherin domains from a human cadherin. Accordingly, human cadherin domains from, for example, human muscle cadherin may be used in the targeting of agents of the present invention, for example but not limited to polypeptide agents of the invention to target muscle cells. The targeting ligand component of the agent polypeptide of the invention may include a naturally occurring or recombinant or engineered ligand, or a fragment thereof, capable of binding the pre-selected target cell.

In another embodiment of the invention, a targeting ligand can consists of at least three, four or five muscle cadherin (M-cadherin) domains, or derivatives or fragments thereof, capable of binding specifically to target cells that express homophilic cadherins. (Shimoyama et al. (1998) J. Biol. Chem. 273(16): 1001 1-10018; Shibata et al. (1997) J. Biol. Chem. 272(8):5236-5270). In some embodiments, the targeting ligand comprises at least three cadherin domains from the extracellular domain of human M-cadherin (or biologically active fragments or derivatives thereof that are capable of binding homophilic M-cadherin), fused to an agent of the present invention.

Further examples of targeting ligands also include, but are not limited to, antibodies and portions thereof that bind a pre-selected cells surface protein with high affinity. By “high affinity” is meant an equilibrium dissociation constant of at least molar, as determined by assay methods known in the art, for example, BiaCore analysis. In one embodiment, the targeting ligand may also comprise one or more immunoglobulin binding domains isolated from antibodies generated against a selected tissue-specific surface protein or target tissue-specific receptor. The term “immunoglobulin or antibody” as used herein refers to a mammalian, including human, polypeptide comprising a framework region from an immunoglobulin gene or fragments thereof that specifically binds and recognizes an antigen, which, in the case of the present invention, is a tissue-specific surface protein, a target tissue-specific receptor, or portion thereof. If the intended targeting fusion polypeptide will be used as a mammalian therapeutic, immunoglobulin binding regions should be derived from the corresponding mammalian immunoglobulins. If the targeting fusion polypeptide is intended for non-therapeutic use, such as for diagnostics and ELISAs, the immunoglobulin binding regions may be derived from either human or non-human mammals, such as mice. The human immunoglobulin genes or gene fragments include the kappa, lambda, alpha, gamma, delta, epsilon, and mu constant regions, as well as the myriad immunoglobulin variable region genes. Light chains are classified as either kappa or lambda. Heavy chains are classified as gamma, mu, alpha, delta, or epsilon, which in turn define the immunoglobulin classes, IgG, 1gM, IgA, IgD, and IgE, respectively. Within each IgG class, there are different isotypes (e.g. IgG1, IgG2, etc.). Typically, the antigen-binding region of an antibody will be the most critical in determining specificity and affinity of binding.

An exemplary immunoglobulin (antibody) structural unit of human IgG, comprises a tetramer. Each tetramer is composed of two identical pairs of polypeptide chains, each pair having one light chain (about 25 kD) and one heavy chain (about 50-70 kD). The N-terminus of each chain defines a variable region of about 100-110 or more amino acids primarily responsible for antigen recognition. The terms “variable light chain” (VL) and variable heavy chain (VH) refer to these light and heavy chains respectively. Antibodies exist as intact immunoglobulins, or as a number of well-characterized fragments produced by digestion with various peptidases. For example, pepsin digests an antibody below the disulfide linkages in the hinge region to produce F(ab)′2, a dimer of Fab which itself is a light chain joined to VH-CH by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the terms immunoglobulin or antibody, as used herein, also includes antibody fragments either produced by the modification of whole antibodies, or those synthesized de novo using recombinant DNA methodologies (e.g., single chain Fv)(scFv)) or those identified using phase display libraries (see, for example, McCafferty et al. (1990) Nature 348:552-554). In addition, the fusion polypeptides of the invention include the variable regions of the heavy (VH) or the light (VL) chains of immunoglobulins, as well as tissue-specific surface protein and target receptor-binding portions thereof. Methods for producing such variable regions are described in Reiter, et al. (1999) J. Mol. Biol. 290:685-698.

Methods for preparing antibodies are known to the art. See, for example, Kohler & Milstein (1975) Nature 256:495-497; Harlow & Lane (1988) Antibodies: a Laboratory Manual, Cold Spring Harbor Lab., Cold Spring Harbor, N.Y.). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity. Techniques for the production of single chain antibodies or recombinant antibodies (U.S. Pat. No. 4,946,778; U.S. Pat. No. 4,816,567) can be adapted to produce antibodies used in the fusion polypeptides and methods of the instant invention. Also, transgenic mice, or other organisms such as other mammals, may be used to express human or humanized antibodies. Alternatively phage display technology can be used to identify antibodies, antibody fragments, such as variable domains, and heteromeric Fab fragments that specifically bind to selected antigens.

Screening and selection of preferred immunoglobulins. (antibodies) can be conducted by a variety of methods known to the art: Initial screening for the presence of monoclonal antibodies specific to a tissue-specific or target receptor may be conducted through the use of ELISA-based methods or phage display, for example. A secondary screen is preferably conducted to identify and select a desired monoclonal antibody for use in construction of the tissue-specific fusion polypeptides of the invention. Secondary screening may be conducted with any suitable method known to the art. One method, termed “Biosensor Modification—Assisted Profiling” (“BiaMAP”) (US patent publication 2004/101920), allows rapid identification of hybridoma clones producing monoclonal antibodies with desired characteristics. More specifically, monoclonal antibodies are sorted into distinct epitope-related groups based on evaluation of antibody: antigen interactions.

### Treatment of Disease and Disorders

The methods of the present invention relate to use of metabolic regulators for the treatment of a number of disorders and diseases, for example but not limited to, muscle associated diseases and disorders, muscle growth, angiogenesis, obesity, insulin sensitivity, insulin-dependent diseases, body weight, muscle mass, fat mass and/or cardiovascular function In one embodiment, a method for treating such disorders comprises administration of an effective amount of an agent that activates or inhibits a metabolic regulator of the present invention, for example an agent that activates or inhibits MSP1, MSP2, MSP3, MPS4 MSP5 or Ins16.

The inventors have discovered that metabolic regulators of the present invention, for example MSP1, MSP2, MSP3, MPS4 MSP5 or Ins16, affect (i) muscle growth (i.e. muscle hypertrophy), for example MSP5 (see Example 6), (ii) angiogenesis, for example MSP3 and MSP5 (see Examples 5 and 6), (iii) glucose sensitivity and insulin sensitivity, for example MSP3 (see Example 5), and (iv) muscle regeneration and satellite cell recruitment, for example Ins16 (see Example 7).

Accordingly, the present invention relates to use of metabolic regulators of the present, for example MSP1, MSP2, MSP3, MPS4 MSP5 or Ins16 or homologues or variants thereof as for the treatment of diseases and/or disorders associated with angiogenesis, insulin sensitivity, muscle degeneration, muscle mass and/or fat mass.

Compositions for Modulating Muscle Mass and/or Hypertrophy.

The inventors discovered the metabolic regulator MSP5 is a myogenic factor and/or muscle hypertrophy factor based on its ability to increase the size of myofibers in vivo and in vitro, increase myofiber size and/or width, and increase protein synthesis as disclosed in Example 6. Accordingly the present invention provides pharmaceutical compositions comprising an agent that functions as an agonist of MSP5, for example an agent that activates MSP5 expression, for example an agent comprising a nucleic acid encoding a MSP5 or functional derivatives to increase muscle hypertrophy and muscle mass in a subject.

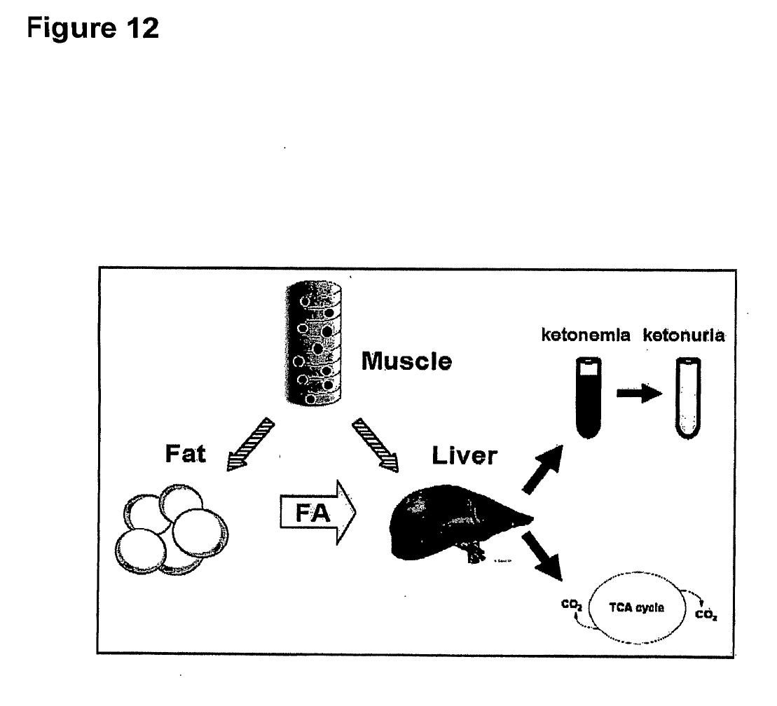

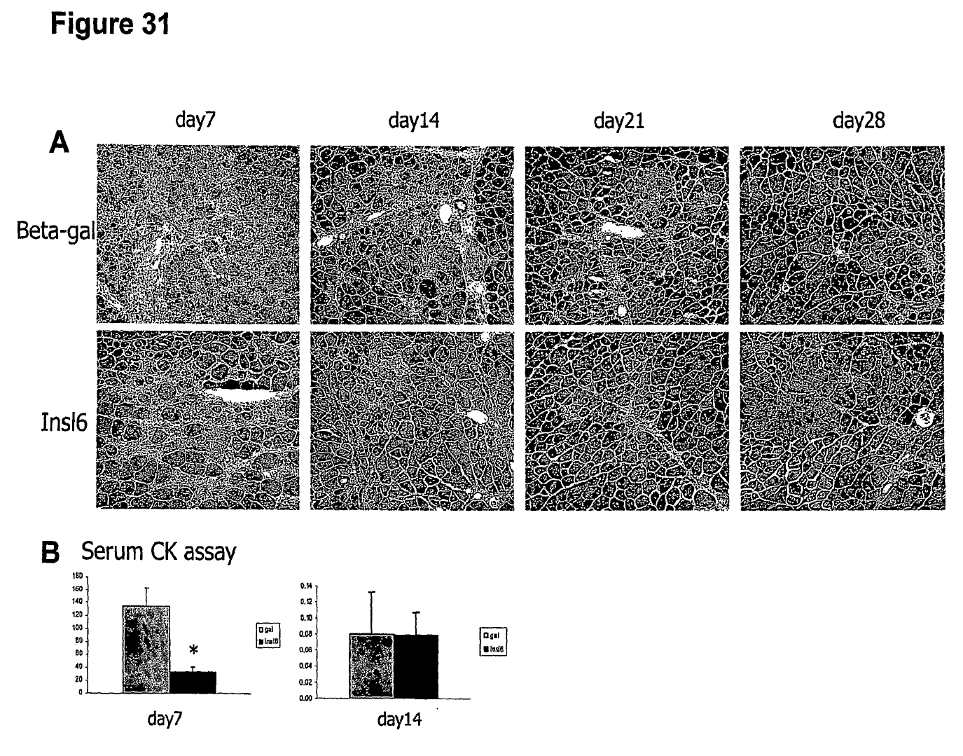

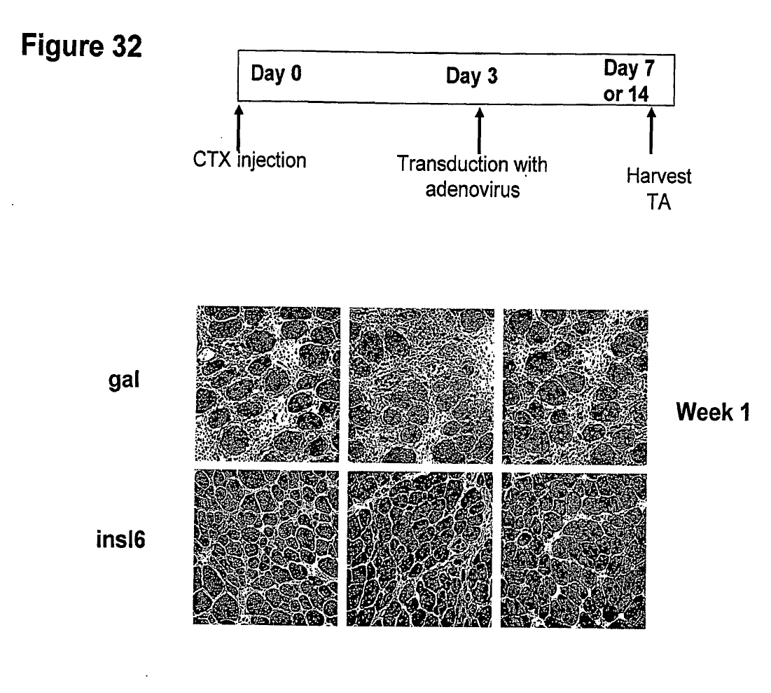

The inventors also discovered Ins16 is identified as muscle regeneration factor based on its ability to increase the number of satellite cells in muscle tissue, as well increasing in BrdU incorporation in satellite cells surrounding myofibrils, and also as an anti-inflammatory factor. Ins16 was further characterized to increase muscle regeneration as it increased immunostaining for the activated satellite marker MyoD, as demonstrated in Example 7. Ins16 was discovered to function as an anti-inflammatory factor based on its ability to decrease the number of inflammatory cells after intramuscular administration of cardiotoxin (CTX), as shown in FIGS. 31 and 32 in Example 7. Additionally, Ins16 improves muscle regeneration after a muscle degeneration injury, for example after intramuscular administration of cardiotoxin (CTX) compared to a muscle in the absence of a gene that promotes muscle growth, for example see FIGS. 38 and 38. Activation of satellite cells in muscle tissue can result in the production of new muscle cells in a subject (For example see FIGS. 12, 30 and 40). Muscle growth, also referred to herein as muscle regeneration refers to the process by which new muscle fibers form from muscle progenitor cells. Accordingly, the present invention provides pharmaceutical compositions comprising a gene and/or gene product (i.e. proteins) of Ins16 or functional derivatives or agonists thereof to increase muscle regeneration and muscle mass in a subject.

One embodiment of the present invention provides methods for increasing muscle mass in an organism. The present invention provides methods and compositions to treat a subject at risk of developing or having muscle atrophy or a subject in need of muscle hypertrophy. In such an embodiment, the method comprises administration of an effective amount of a pharmaceutical composition comprising an agent targeting a metabolic regulator MSP5 or functional derivatives or agonists thereof to a subject in need thereof. In some embodiments, the pharmaceutical compositions comprise an agonist of MSP5. In an alternative embodiment, the present invention provides a means to treat a subject with, or at risk of muscle atrophy by administering a pharmaceutical composition comprising an agent that functions as an agonist of MSP5, for example but not limited to an agent that is a nucleic acid encoding MSP5 (SEQ ID NO: 12) or a homologue or fragment thereof to the subject. Accordingly, the present invention provides methods to increase muscle growth by increasing muscle hypertrophy or inhibiting atrophy in a subject in need thereof.

In another embodiment, the present invention provides methods for increasing muscle regeneration in an organism. The present invention provides methods and compositions to treat a subject at risk of developing or having muscle degeneration or a subject in need of muscle regeneration. In such an embodiment, the method comprises administration of an effective amount of a pharmaceutical composition comprising a gene and/or gene product (i.e. proteins) of Ins16 or functional derivatives or agonists thereof to a subject in need thereof. In some embodiments, the pharmaceutical compositions comprise an agonist of Ins16. In an alternative embodiment, the present invention provides a means to treat a subject with, or at risk of muscle atrophy by administering a pharmaceutical composition comprising a nucleic acid encoding Ins16 (SEQ ID: NO:20) or a homologue or fragment thereof to the subject. Accordingly, the present invention provides methods to increase muscle mass by increase satellite cell recruitment and increasing muscle regeneration in a subject in need thereof.

A pharmaceutical composition comprising an agent that an agent that functions as an agonist or MSP5 and/or Ins16 or functional derivatives or agonists thereof, will result in muscle growth and/or muscle regeneration. An increase in the number of new fibers by at least 1%, more preferably by at least 20%, and most preferably by at least 50% occurs in the presence of Ins16 as compared to the absence of Ins16. As used herein, the term “muscle growth” refers to the increase in the fiber size and/or an increase in the number of fibers. The growth of muscle may be measured by an increase in wet weight, an increase in protein content, an increase in the number of muscle fibers; an increase in muscle fiber diameter; etc. An increase in growth of a muscle fiber can be defined as an increase in the diameter where the diameter is defined as the minor axis of ellipsis of the cross section.

In some embodiments, the muscle growth induced by Ins16 and/or MSP5 can be compared with the muscle growth associated with a positive control. In some embodiments, the positive control is an inhibitor of myostatin, for example a neutralizing antibody of myostatin or inhibitory nucleic acid of myostain, for example RNAi of myostatin or a dominant negative form of myostatin.

Muscle growth may also be monitored by the mitotic index of muscle. For example, cells may be exposed to a labeling agent for a time equivalent to two doubling times. The mitotic index is the fraction of cells in the culture which have labeled nuclei when grown in the presence of a tracer which only incorporates during S phase (i.e., BrdU) and the doubling time is defined as the average S time required for the number of cells in the culture to increase by a factor of two. Productive muscle regeneration may be also monitored by an increase in muscle strength and agility.

Muscle growth may also be measured by quantitation of myogenesis, i.e. fusion of myoblasts to yield myotubes. An effect on myogenesis results in an increase in the fusion of myoblasts and the enablement of the muscle differentiation program. For example, the myogenesis may be measured by the fraction of nuclei present in multinucleated cells in relative to the total number of nuclei present. Myogenesis may also be determined by assaying the number of nuclei per area in myotubes or by measurement of the levels of muscle specific protein by Western analysis.

The survival of muscle fibers may refer to the prevention of loss of muscle fibers as evidenced by necrosis or apoptosis or the prevention of other mechanisms of muscle fiber loss. Muscles can be lost from injury, atrophy, and the like, where atrophy of muscle refers to a significant loss in muscle fiber girth.

Such disorders where administration of pharmaceutical compositions comprising MSP3 and/or Ins16 is useful is when the subject is in need of increasing muscle mass, either by increasing muscle hypertrophy or increasing muscle regeneration, or both, where the pharmaceutical compositions comprising MSP3 and/or Ins16 would ameliorate the disease symptoms. Such diseases include for example, where the subject has muscle atrophy or muscle degeneration. For example, a decrease in muscle mass, or atrophy, is associated with various physiological and pathological states. For example, muscle atrophy can result from denervation due to nerve trauma; degenerative, metabolic or inflammatory neuropathy, e.g. Guillian-Barre syndrome; peripheral neuropathy; or nerve damage caused by environmental toxins or drugs.

Muscle atrophy may also result from denervation due to a motor neuropathy including, for example, adult motor neuron disease, such as Amyotrophic Lateral Sclerosis (ALS or Lou Gehrig's disease); infantile and juvenile spinal muscular atrophies; and autoimmune motor neuropathy with multifocal conductor block. Muscle atrophy may also result from chronic disease resulting from, for example, paralysis due to stroke or spinal cord injury; skeletal immobilization due to trauma, such as, for example, fracture, ligament or tendon injury, sprain or dislocation; or prolonged bed rest. Metabolic stress or nutritional insufficiency, which may also result in muscle atrophy, include the cachexia of cancer and other chronic illnesses including AIDS, fasting or rhabdomyolysis, and endocrine disorders such as disorders of the thyroid gland and diabetes.

Muscle atrophy may also be due to muscular dystrophy syndromes such as Duchenne, Becker, myotonic, fascioscapulohumeral, Emery-Dreifuss, oculopharyngeal, scapulohumeral, limb girdle, and congenital types, as well as the dystrophy known as Hereditary Distal Myopathy. Muscle atrophy may also be due to a congenital myopathy, such as benign congenital hypotonia, central core disease, nemalene myopathy, and myotubular (centronuclear) myopathy. Muscle atrophy also occurs during the aging process. Muscle atrophy in various pathological states is associated with enhanced proteolysis and decreased production of muscle proteins.

Other examples of such diseases where the subject has muscle atrophy or muscle degeneration are, for example but not limited to, Muscular disorders include muscular dystrophy, Duchenne dystrophy; Becker muscular dystrophy; congenital myopathies including nemaline myopathy or myopathy caused by mutations in the gene for the ryanodine receptor; mitochondrial myopathies due to mutations in both mitochondrial and nuclear-encoded genes including progressive external opthalmoplegia, the Kearns-Sayre syndrome, the MELAS, the MERFF syndrome, infantile myopathy; glycogen storage diseases of muscle including Pompe's disease; channelopathies; myotonic dystrophy (Steinert's disease); myotonia congenita (Thomsen's disease); familial periodic paralysis including hypokalemic (due to mutation in the dihydropyridine receptor-associated calcium channel gene on chromosome 1 q) and hyperkalemic form (due to mutation in SCN4A on chromosome 17q).

Administration of a pharmaceutical compositions comprising an agent that functions as an agonist to MSP5 and/or Ins16 is also useful is when the subject is in need of increasing muscle mass in settings of more general body mass wasting, such as for example cachexia. Cachexia is a condition causing body mass loss, including, but not limited to, muscle mass. Settings of cachexia include cancer-induced cachexia, Al OS-induced cachexia, sepsis-induced cachexia, renal failure-induced cachexia, and congestive heart failure. Also, there is growth retardation in many settings, including thalassaemia, which causes short stature. Short stature in general would be a setting for an administration of pharmaceutical compositions comprising an agent that functions as an agonist to MSP5 and/or Ins16.

Muscle atrophy also occurs during the aging process. Muscle atrophy in various pathological states is associated with enhanced proteolysis and decreased production of muscle proteins. Administration of pharmaceutical compositions comprising MSP5 and/or Ins16 may also be useful in treating subjects suffering from growth hormone deficiencies, tissue wasting including burns, skeletal trauma, infection, cancer, cystic fibrosis, Duchenne muscular dystrophy, Becker dystrophy, autosomal recessive dystrophy, polymyositis, as well as myopathies and AIDS (U.S. Pat. No. 5,622,932).

Compositions for Modulating Glucose and/or Insulin Sensitivity and Body Weight.

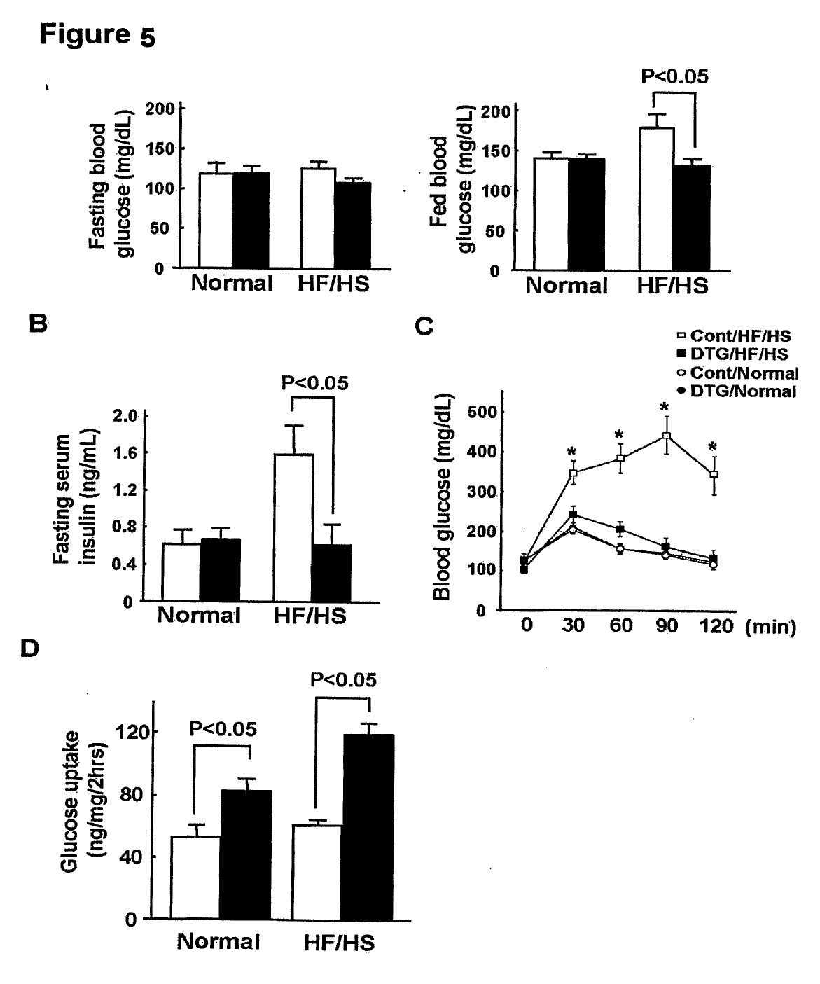

The inventors discovered that MSP3 is metabolic regulator, for example a regulator of glucose sensitivity and/or insulin sensitivity on the basis of its ability to regulate glucose and/or insulin sensitivity in a mouse model of obesity. In particular MSP3 was shown to increase sensitivity to glucose and insulin as determined by measuring blood glucose and insulin serum level in a glucose tolerance test (GTT) in a mouse model of obesity, where the mice are fed a high fat, high sucrose diet (HF/HS) diet to induce obesity. MSP3 treated mice fed a HF/HS diet have a lower glucose blood level after injection and/or reduced fasting serum glucose and/or insulin level as compared to a mice in the absence of MPS3, leading to the discovery that MSP3 increases sensitivity to glucose and/or insulin, for example as discussed in Example 1 and FIGS. 5, 17 and 18.

Accordingly the present invention provides pharmaceutical compositions comprising agent for the gene and/or gene product (i.e. proteins) of MSP3 or functional derivatives or agonists thereof to reduce insulin insensitivity or insulin tolerance in a subject. Accordingly, agents that function as agonists to MSP3 or functional derivatives thereof are useful in the present invention to reduce obesity and/or fat mass.

In another embodiment of the present invention relates to methods for regulating glucose and/or insulin insensitivity in an organism. The present invention provides methods and compositions to treat a subject at risk of developing or having insulin and/or glucose insensitivity or a subject in need of glucose regulation or metabolic regulation, for example a subject with a disorder involving insulin resistance. In such an embodiment, the method comprises administration of an effective amount of a pharmaceutical composition comprising an agent that functions as an agonist for example an agent that is a gene and/or gene product (i.e. proteins) of MSP3 or functional derivatives or agonists thereof to a subject in need thereof. In some embodiments, the pharmaceutical compositions comprise an agonist of MSP3. In an alternative embodiment, the present invention provides a means to treat a subject with, or at risk of muscle atrophy by administering a pharmaceutical composition comprising a nucleic acid encoding MSP3 (SEQ ID: NO1) or a homologue or fragment thereof to the subject. Accordingly, the present invention provides methods to increase glucose sensitivity and/or increase insulin sensitivity in a subject in need thereof.

Such disorders where administration of pharmaceutical compositions comprising agents that function as agonists of MSP3 is useful is when the subject has, or is at risk of developing an insulin-resistant disorder and/or has abnormal glucose intolerance, where the pharmaceutical compositions comprising MSP3 would ameliorate the disease symptoms. Such diseases include for example, diseases resulting from the failure of the normal metabolic response of peripheral tissues (insensitivity) to the action of exogenous insulin (i.e. it is a condition where in the presence of insulin produces a subnormal biological response. In clinical terms, insulin resistance is present when normal or elevated blood glucose levels persist in the face of normal or elevated levels of insulin. It represents, in essence, a glycogen synthesis inhibition, by which either basal or insulin stimulated glycogen synthesis, or both, are reduced below normal levels. Insulin resistance plays a major role in Type 2 diabetes, as demonstrated by the fact that the hyperglycemia present in Type 2 diabetes can sometimes be reversed by diet or weight loss sufficient, apparently, to restore the sensitivity of peripheral tissues to insulin.

In some embodiments, pharmaceutical compositions comprising agents that function as agonists of MSP3 is useful is when the subject has, or is at risk of developing abnormal glucose intolerance, which is associated with many disorders in which insulin resistance plays a key role, for example but not limited to, obesity, diabetes mellitus, ovarian hyperandrogenism, and hypertension, insulin dependent and non-insulin dependent diabetes mellitus.

In some embodiments, pharmaceutical compositions comprising agents that function as agonists of MSP3 are useful is treating subjects with symptoms and complications of diabetes, for example but not limited to, hyperglycemia, unsatisfactory glycemic control, ketoacidosis, insulin resistance, elevated growth hormone levels, elevated levels of glycosylated hemoglobin and advanced glycosylation end-products (AGE), dawn phenomenon, unsatisfactory lipid profile, vascular disease (e.g., atherosclerosis), microvascular disease, retinal disorders (e.g., proliferative diabetic retinopathy), renal disorders, neuropathy, complications of pregnancy (e.g., premature termination and birth defects) and the like. Included in the definition of treatment are such end points as, for example, increase in insulin sensitivity, reduction in insulin dosing while maintaining glycemic control, decrease in HbA1c, improved glycemic control, reduced vascular, renal, neural, retinal, and other diabetic complications, prevention or reduction of the “dawn phenomenon”, improved lipid profile, reduced complications of pregnancy, and reduced ketoacidosis.

Compositions for Modulating Angiogenesis.

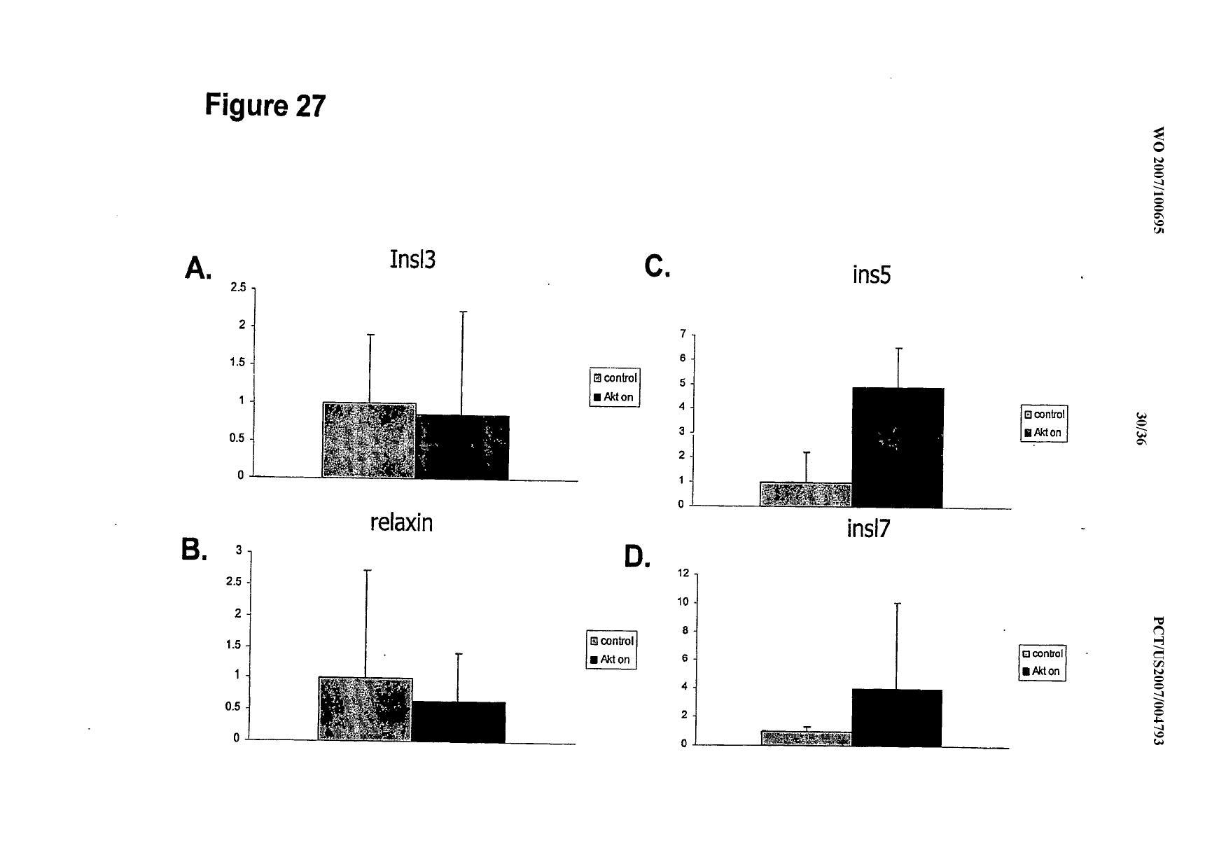

The inventor have discovered that both MSP3 and MSP5 promote angiogenesis based on their ability to promote revascularization in a mouse model of ischemia, as disclosed in Examples 5 and 6 (see also FIG. 27) where mice are subjected to unilateral hind limb surgery (J. Biol. Chem. 2004; 279:28670-28674; Circ. Res. 2005; 96(8):838-846; Circ. Res. 2006; 98(2):254-61).

Accordingly, the present invention provides pharmaceutical compositions comprising an agent that functions as an agonist of MSP3 and/or activates MSP3 and/or MSP5 or functional derivatives or agonists thereof to promote angiogenesis in a subject in need thereof. In alternative embodiments, the present invention provides pharmaceutical compositions comprising an agent that functions as an inhibitor or antagonist to MSP3 and/or MSP5 gene and/or gene product or functional derivatives thereof to inhibit angiogenesis in a subject in need thereof.

In yet another embodiment, the present invention relates to methods for increasing angiogenesis in an organism. The present invention provides methods and compositions to treat a subject at risk of developing ischemia or having lack of angiogenesis and/or blood vessel formation or a subject in need of angiogenesis. In such an embodiment, the method comprises administration of an effective amount of a pharmaceutical composition comprising an agent that functions as an agonist for example but not limited to a gene and/or gene product (i.e. proteins) of MSP3 or functional derivatives or agonists thereof to a subject in need thereof. In an alternative embodiment, the method comprises administration of an effective amount of a pharmaceutical composition comprising a gene and/or gene product (i.e. proteins) of MSP5 or functional derivatives or agonists thereof to a subject in need thereof. In further embodiments, the pharmaceutical composition can comprise genes and/or gene products (i.e. proteins) of MSP3 and MSP5 or functional derivatives or agonists thereof. In some embodiments, the pharmaceutical compositions comprise an agonist of MSP3 and/or MSP5. In an alternative embodiment, the present invention provides a means to treat a subject with, or at risk of muscle atrophy by administering a pharmaceutical composition comprising a nucleic acid encoding MSP3 (SEQ ID: NO1) and/or MSP5 (SEQ ID NO: 12) or homologues or fragments thereof to the subject. Accordingly, the present invention provides methods to increase angiogenesis in a subject in need thereof.

Diseases and disorders where it is desirable to increase angiogenesis, and thus administration of a pharmaceutical composition comprising an agent that functions as an agonist of MSP3 and/or MSP5 of the present invention, include, for example but not limited to a subject suffering from, or at risk of having ischemia and/or acute myocardial infarction. The administration of a pharmaceutical composition comprising an agent that functions as an agonist of MSP3 and/or MSP5 either prior to, concurrent with, or post ischemia and/or myocardial infarction reduces necrosis and inflammation in the myocardium and preserves organ function.

As used herein, the term “ischemia” refers to any localized tissue ischemia due to reduction of the inflow of blood. In some embodiments the ischemia is tissue ischemia or brain ischemia, for example a blood clot to a tissue or to the brain in the case of a stroke. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue. Symptoms of myocardial infarction include pain, fullness, and/or squeezing sensation of the chest, jaw pain, toothache, headache, shortness of breath, nausea, vomiting, and/or general epigastric (upper middle abdomen) discomfort; sweating, heartburn and/or indigestion, arm pain (more commonly the left arm, but may be either arm), upper back pain, and general malaise (vague feeling of illness).

Acute myocardial infarction (MI) strikes a majority of sufferers without prior warning and in the absence of clinically detectable predisposing risk factors (Braunwald E. Heart Disease: a Textbook of Cardiovascular Medicine. 1997). When patients come to the intensive unit in a hospital showing symptoms of acute MI, the diagnosis for acute MI can be made by monitoring (1) an increase in the plasma concentration of cardiac enzymes and (2) either a typical clinical presentation and/or typical ECG changes. Either of the following parameters will fulfill the requirement for an increase in cardiac enzymes: Total creatine-kinase (CK) at least 2 times the upper limit of the normal range, or CK-MB (muscle-brain) above the upper limit of the normal range and at least 5% of the normal CK. If total CK or CK-MB is not available, the following will be accepted in the fulfillment of the criteria for acute MI: Troponin T at least 3 times the upper limit of the normal range; Troponin I at least 3 times the upper limit of the normal range. The use of Troponin T as a serum marker for MI is disclosed in V. V. Murthy and A. Karmen, J. Clin. Labor. Analys. 11: 125-128 (1997). The analytical performance and clinical utility if a sensitive immunoassay for determination of cardiac Troponin I can be taken from E. Davies et al. Clin. Biochem. 30: 479-490 (1997). Typical ECG changes include evolving ST-segment or T-wave changes in two or more contiguous ECG leads, the development of new pathological Q/QS waves in two or more contiguous ECG leads, or the development of new left bundle branch block.

In an alternative embodiment, the present invention provides methods and compositions to treat a subject in need of reduced angiogenesis or a subject at risk of developing excessive angiogenesis, for example a subject with or at risk of developing cancer. In such an embodiment, the method comprises administration of an effective amount of a pharmaceutical composition comprising an agent that functions as an antagonist to gene and/or gene product (i.e. proteins) of MSP3 and/or MSP5 or functional derivatives thereof, to a subject in need thereof. Accordingly, the present invention provides methods to decrease angiogenesis in a subject in need thereof.

Diseases and disorders where it is desirable to decrease angiogenesis, and thus administration of a pharmaceutical composition comprising an agent that functions as an inhibitor to MSP3 and/or MSP5 of the present invention, include, for example but not limited to a subject suffering from or at risk of developing cancer or a tumor. Other diseases where administration of a pharmaceutical composition comprises an agent that inhibits the activity and/or expression of MSP3 and/or MSP5 to inhibit angiogenesis include, for example but not limited to rheumatoid arthritis, retinopathies, diabetic retinopathy, inflammatory diseases, restenosis, and the like.

Other diseases and/or disorders where administration of a pharmaceutical composition comprising an inhibitor to MSP3 and/or MSP5 of the present invention would be useful in reducing angiogenesis, include, for example angiogenic diseases, such as but not limited to, inflammatory disorders such as immune and non-immune inflammation, chronic articular rheumatism and psoriasis, disorders associated with inappropriate or inopportune invasion of vessels such as diabetic retinopathy, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi sarcoma and the like cancers which require neovascularization to support tumor growth.

Compositions for Modeling Obesity.

The inventors discovered numerous metabolic regulators that affect muscle mass and insulin sensitivity, for example but not limited to MSP5, Ins16 and MSP3. Accordingly, also encompassed in the methods of the present invention is administration to a subject a pharmaceutical composition comprising an agent that functions as an antagonist and/or inhibits the activity of at least one metabolic regulator of the present invention, for example an agent that functions to inhibit MSP5 and/or Ins16, and/or an agent that functions as an agonist of MPS3, where the subject is in need of decreased muscle mass, or decreased weight or is suffering from or at risk of developing obesity.

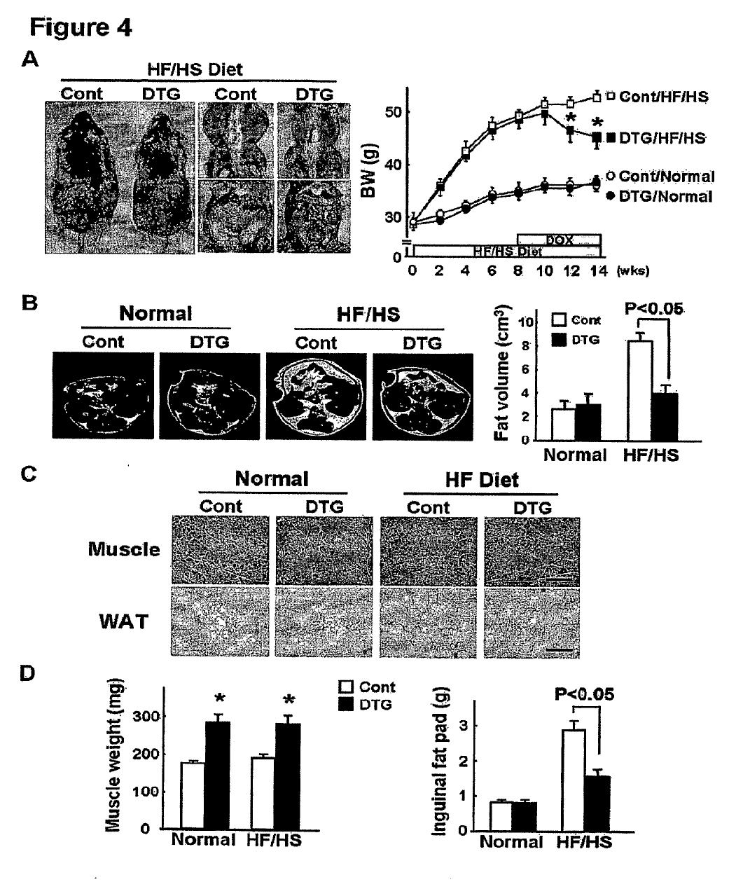

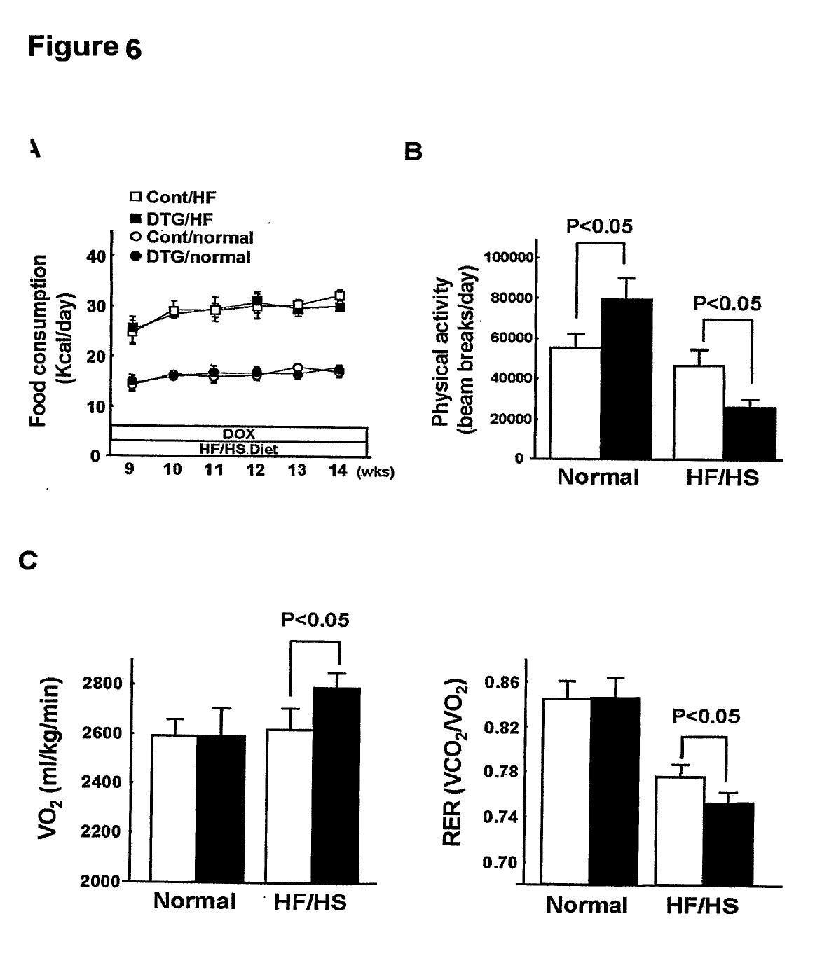

Exemplary methods to analyze the ability of an agent to reduce body weight and fat mass are described in Example 1, including assessment of total body weight, levels of excess fat by MRI, quantification of adipose cell size, muscle weight, and inguinal fat pad weight in an animal model of obesity, for example mice fed a high fat, high sucrose diet (HF/HS) diet to induce obesity in the presence or absence of an agent that targets at least one metabolic regulator of the present invention, for example MSP1, MPS2, MSP3, MPS4, MSP5 or Ins16. An agent is identified to reduce body mass and/or fat mass if in an obesity animal model, for example mice fed a HF/HS diet, the animals have a lower body weight and/or lower excessive fat as detected by, for example MRI, and/or smaller adipose cell size and/or decreased muscle weight and/or decreased inguinal fat pad weight as compared to a mice in the absence of a gene that functions to increase sensitivity to glucose and/or insulin, for example as discussed in Example 1 and FIG. 4. Further characterization to assess the ability of an agent to reduce body weight and/or reverse excessive fat accumulation is described in Example 1, (see FIG. 6) can be done by analyzing the energy balance, such as food intake and energy expenditure an animal model of obesity, for example mice fed a high fat, high sucrose diet (HF/HS) diet to induce obesity in the presence or absence of the gene or gene product (for example viral mediated expression of the gene) as discussed in FIG. 6. Measurements of energy intake, such as food and water intake, energy expenditure by whole body O₂ consumption (VO₂) and Respiratory exchange ratio (RER) which reflects the ratio of carbohydrate to fatty acid oxidation can be done, as well as quantitative analysis, for example by quantitative PCR or QRT-PCR of genes associated with fatty acid oxidation and mitochondrial biogenesis in the skeletal muscle and/or liver. In addition, liver morphology and lipid oxidative function can be analyzed, as well as the effect on HF/HS diet-induced lipid deposition in the liver, as well as serum ketone bodies, which synthesized in the liver and can be used as an indirect marker of hepatic fatty acid oxidation, as well as quantitative analysis of molecules that stimulate fatty acid oxidation in the liver, for example HNF4α, L-CPT1 and PGC1-α. A gene and/or gene product is identified as being capable of reducing body mass and/or fat mass if, in an obesity animal model, for example mice fed a HF/HS diet, the animals have an increased VO2 and/or decreased RER indicating a greater ratio of use of fatty acid as a fuel source, and/or decreased lipid deposition, and/or increased fatty acid oxidation and/or increased serum ketone bodies in the liver and increased expression of markers for fatty acid oxidation in the liver and/or skeletal muscle as compared to a mice in the absence of a gene that functions to increase sensitivity to glucose and/or insulin, for example as discussed in Example 1 and FIG. 4.

In yet another embodiment, the present invention relates to methods for treating obesity in an organism. The method comprises administration of a pharmaceutical composition comprising an agent that targets at least one metabolic regulator of the present invention, for example MSP1, MPS2, MSP3, MPS4, MSP5 or Ins16 and characterized to function to decrease muscle mass and/or fat mass and/or increase VO2 and/or increase fatty acid.

In another aspect, the invention features therapeutic methods for the treatment of a disease or condition, comprising administering an effective amount of a pharmaceutical composition comprising an agent targeting a metabolic regulator of the present invention, for example MSP1, MPS2, MSP3, MPS4, MSP5 or Ins16, or functional derivatives or variants thereof of the invention, to a subject in need thereof, or a subject at risk for development of that disease or condition. When the disease or condition is a muscle condition, such as atrophy, the method of the present invention comprises administering an effective amount of an pharmaceutical composition comprising an agent that functions as an agonist, for example activates MSP3, and/or MSP5 and/or Ins16, or functional derivatives or variants thereof, to a subject in need thereof, wherein the muscle-related disease or condition is ameliorated or inhibited. The muscle-related condition or disorder treated by the pharmaceutical compositions of the invention may arise from a number of sources, including for example: denervation; degenerative, metabolic or inflammatory neuropathy; infantile and juvenile spinal muscular atrophies; autoimmune motor neuropathy; from chronic disease, including cachexia resulting from cancer, AIDS, fasting or rhabdomyolysis; and from muscular dystrophy syndromes such as Duchenne.

The methods of the present invention are also useful to treat any condition which is results from a deficiency in the metabolic regulators of the present invention, for example any condition due to a deficiency in MSP1, MPS2, MSP3, MPS4, MSP5 or Ins16 or which may be improved by increased levels of MSP1, MPS2, MSP3, MPS4, MSP5 or Ins16, the method comprising administering an effective amount of pharmaceutical composition comprising an agent that that functions as an agonist of at least one metabolic regulator, for example an agent that activates at least one of MSP1, MPS2, MSP3, MPS4, MSP5 or Ins16 or homologues thereof, where the effective amount is sufficient to alleviate at least one or some of the symptoms associated with the lack of one of the metabolic regulators, for example symptoms associated with the lack of MSP1, MPS2, MSP3, MPS4, MSP5 or Ins16. Such diseases include, for example but are not limited to dwarfism and heart disease, for example but are not limited to, improved heart tissue survival following myocardial infarction.

In another alternative embodiment, the present invention also encompasses methods useful for the treatment of any condition which is results from an elevated level of the metabolic regulators of the present invention, for example any condition due to increased expression and/or activity of MSP1, MPS2, MSP3, MPS4, MSP5 or Ins16 or which may be improved by reduction in levels of MSP1, MPS2, MSP3, MPS4, MSP5 or Ins16, the method comprising administering an effective amount of pharmaceutical composition comprising an agent that functions as an antagonist or inhibitor of at least one metabolic regulator, for example an agent that inhibits at least one of MSP1, MPS2, MSP3, MPS4, MSP5 or Ins16 or homologues thereof, where the effective amount is sufficient to alleviate at least one or some of the symptoms associated with the increased expression and/or activity of one of the metabolic regulators, for example MSP1, MPS2, MSP3, MPS4, MSP5 or Ins16. Such diseases include, for example but are not limited to obesity and cancer.

### Administration of Pharmaceutical Compositions

In some embodiments, agents targeting the metabolic regulators of the present invention are administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including, but not limited to, improved survival rate or more rapid recovery, or improvement or elimination of symptoms and other indicators as are selected as appropriate measures by those skilled in the art.

It should be noted that agents targeting the metabolic regulators of the present invention can be administered as a compound or as a pharmaceutically acceptable salt and can be administered alone or as an active ingredient in combination with pharmaceutically acceptable carriers, diluents, adjuvants and vehicles.

It is also noted that humans are treated generally longer than the mice or other experimental animals exemplified herein, which treatment has a length proportional to the length of the disease process and drug effectiveness. The doses may be single doses or multiple doses over a period of several days, but single doses are preferred.

When administering the agents targeting the metabolic regulators of the present invention parenterally, it will generally be formulated in a unit dosage injectable form (e.g., solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (e.g., glycerol, propylene glycol, liquid polyethylene glycol), suitable mixtures thereof, and vegetable oils.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

After formulation with an appropriate pharmaceutically acceptable carrier in a desired dosage, the pharmaceutical compositions of this invention can be administered to a subject. The pharmaceutical compositions of this invention can be administered to a subject using any suitable means. In general, suitable means of administration include, but are not limited to, topical, oral, parenteral (e.g., intravenous, subcutaneous or intramuscular), rectal, intracisternal, intravaginal, intraperitoneal, ocular, or nasal routes.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systematically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

When the compounds of the present invention, for example Compound (I) are administered as pharmaceuticals, to humans and mammals, they can be given per se or as a pharmaceutical composition containing, for example, 0.1 to 99.5% (more preferably, 0.5 to 90%) of active ingredient, i.e., at least one compound I and/or derivative thereof, in combination with a pharmaceutically acceptable carrier.

In general, a suitable daily dose of a compound of the invention will be that amount of the compound which is the lowest dose effective to produce a therapeutic effect. Such an effective dose will generally depend upon the factors described above.

If desired, the effective daily dose of the active compound may be administered as two, three, four, five, six or more sub-doses administered separately at appropriate intervals throughout the day, optionally, in unit dosage forms.

The pharmaceutical compositions of the invention include a “therapeutically effective amount” or a “prophylactically effective amount” of one or more of the resolvins and/or protectins or analogues thereof of the invention. A “therapeutically effective amount” refers to an amount effective, at dosages and for periods of time necessary, to achieve the desired therapeutic result, e.g., a diminishment or prevention of effects associated with various disease states or conditions. A therapeutically effective amount of the resolvins and/or protectins or analogues thereof may vary according to factors such as the disease state, age, sex, and weight of the subject, and the ability of the therapeutic compound to elicit a desired response in the subject. A therapeutically effective amount is also one in which any toxic or detrimental effects of the therapeutic agent are outweighed by the therapeutically beneficial effects.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount. A prophylactically or therapeutically effective amount is also one in which any toxic or detrimental effects of the compound are outweighed by the beneficial effects.

Dosage regimens may be adjusted to provide the optimum desired response (e.g. a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Actual dosage levels of the active ingredients in the pharmaceutical compositions of this invention may be varied so as to obtain an amount of the active ingredient which is effective to achieve the desired therapeutic response for a particular subject, composition, and mode of administration, without being toxic to the patient.

“Dosage unit” form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the compound, for example compound (I) or derivative thereof and the particular therapeutic or prophylactic effect to be achieved, and (b) the limitations inherent in the art of compounding such an active compound for the treatment of sensitivity in individuals.

The therapeutically effective amount can be estimated initially either in cell culture assays or in animal models, usually mice, rabbits, dogs, or pigs. The animal model is also used to achieve a desirable concentration range and route of administration. Such information can then be used to determine useful doses and routes for administration in other subjects. Generally, the therapeutically effective amount is dependent of the desired therapeutic effect. For example, the therapeutically effective amount of an agent activating MSP3 is sufficient to increase blood vessel growth and/or angliogeneis in a model of Ischemia, for example as disclosed in Example 5 and 6, and/or sufficient to increase glucose sensitivity in a GTT test in a model of obesity, for example as disclosed in Example 5 or reduce weight in a model of obesity. As an illustrative example only, the therapeutically effective amount of an agent activating MSP5 is sufficient to increase blood vessel growth and/or angiogenesis in a model of Ischemia, for example as disclosed in Example 5 and 6, and/or sufficient to increase muscle mass and/or muscle hypertrophy as disclosed in Example 1 and Example 6. As another illustrative example only, the therapeutically effective amount of an agent activating Ins16 is sufficient to increase muscle mass and/or muscle regeneration in a model of muscle degeneration, for example intramuscular CTX injection as disclosed in Example 7, or a model of muscular dystrophy.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Proper fluidity can be maintained, for example, by the use of a coating such as lecithin, by the maintenance of the required particle size in the case of dispersion and by the use of surfactants. Non-aqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, e.g., parabens, chlorobutanol, phenol and sorbic acid. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicles, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include those presented in U.S. Pat. Nos. 5,225,182; 5,169,383; 5,167,616; 4,959,217; 4,925,678; 4,487,603; 4,486,194; 4,447,233; 4,447,224; 4,439,196 and 4,475,196. Other such implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques that deliver the compound orally or intravenously and retain the biological activity are preferred.

In another embodiment, the pharmaceutically acceptable formulations comprise lipid-based formulations. Any of the known lipid-based drug delivery systems can be used in the practice of the invention. For instance, multivesicular liposomes, multilamellar liposomes and unilamellar liposomes can all be used so long as a sustained release rate of the encapsulated active compound can be established. Methods of making controlled release multivesicular liposome drug delivery systems are described in PCT Application Publication Nos: WO 9703652, WO 9513796, and WO 9423697, the contents of which are incorporated herein by reference.

The composition of the synthetic membrane vesicle is usually a combination of phospholipids, usually in combination with steroids, especially cholesterol. Other phospholipids or other lipids may also be used. Examples of lipids useful in synthetic membrane vesicle production include phosphatidylglycerols, phosphatidylcholines, phosphatidylserines, phosphatidylethanolamines, sphingolipids, cerebrosides, and gangliosides, with preferable embodiments including egg phosphatidylcholine, dipalmitoylphosphatidylcholine, distearoylphosphatidyleholine, dioleoylphosphatidylcholine, dipalmitoylphosphatidylglycerol, and dioleoylphosphatidylglycerol.

In preparing lipid-based vesicles containing an active compound such variables as the efficiency of active compound encapsulation, liability of the active compound, homogeneity and size of the resulting population of vesicles, active compound-to-lipid ratio, permeability, instability of the preparation, and pharmaceutical acceptability of the formulation should be considered.

Prior to introduction, the formulations can be sterilized, by any of the numerous available techniques of the art, such as with gamma radiation or electron beam sterilization.

When the agents are delivered to a patient, they can be administered by any suitable route, including, for example, orally (e.g., in capsules, suspensions or tablets) or by parenteral administration. Parenteral administration can include, for example, intramuscular, intravenous, intraarticular, intraarterial, intrathecal, subcutaneous, or intraperitoneal administration. The agent can also be administered orally, transdermally, topically, by inhalation (e.g., intrabronchial, intranasal, oral inhalation or intranasal drops) or rectally. Administration can be local or systemic as indicated. Agents can also be delivered using viral vectors, which are well known to those skilled in the art.

The pharmaceutically acceptable formulations can be suspended in aqueous vehicles and introduced through conventional hypodermic needles or using infusion pumps.

### Pharmaceutical Compositions

In another embodiment of the invention, pharmaceutical compositions containing one or more agent targeting the metabolic regulators are disclosed, in any combination, and in conjunction with any other therapeutic agent. For purpose of administration, an agent targeting the metabolic regulators, for example an agent targeting MSP1, MSP2, MPS3, MPS4, MPS5 and/or Ins16 or functional derivatives thereof is preferably formulated as a pharmaceutical composition. Pharmaceutical compositions of the present invention comprise a compound of this invention and a pharmaceutically acceptable carrier, wherein the compound is present in the composition in an amount which is effective to treat the condition of interest. Appropriate concentrations and dosages can be readily determined by one skilled in the art.

Pharmaceutically acceptable carriers are familiar to those skilled in the art. For compositions formulated as liquid solutions, acceptable carriers include saline and sterile water, and may optionally include antioxidants, buffers, bacteriostats and other common additives. The compositions can also be formulated as pills, capsules, granules, or tablets which contain, in addition to a compound of this invention, diluents, dispersing and surface active agents, binders, and lubricants. One skilled in this art may further formulate the compounds of this invention in an appropriate manner, and in accordance with accepted practices, such as those disclosed in Remington's Pharmaceutical Sciences, Gennaro, Ed., Mack Publishing Co., Easton, Pa. 1990.

While it is possible for an agent targeting the metabolic regulators, for example an agent targeting MSP1, MSP2, MPS3, MPS4, MPS5 and/or Ins16, to be administered alone, it is preferable to administer agents activating and/or suppressing activity of MSP3 or MSP5 or Ins16 or functional derivatives as a pharmaceutical composition.

Formulations of the invention can be prepared by a number or means known to persons skilled in the art. In some embodiments the formulations can be prepared by combining (i) an agent targeting the metabolic regulators, for example an agent targeting MSP1, MSP2, MPS3, MPS4, MPS5 and/or Ins16 or functional derivatives thereof in an amount sufficient to provide a plurality of therapeutically effective doses; (ii) the water addition in an amount effective to stabilize each of the formulations; (iii) the propellant in an amount sufficient to propel a plurality of doses from an aerosol canister; and (iv) any further optional components e.g. ethanol as a cosolvent; and dispersing the components. The components can be dispersed using a conventional mixer or homogenizer, by shaking, or by ultrasonic energy. Bulk formulation can be transferred to smaller individual aerosol vials by using valve to valve transfer methods, pressure filling or by using conventional cold-fill methods. It is not required that a stabilizer used in a suspension aerosol formulation be soluble in the propellant. Those that are not sufficiently soluble can be coated onto the drug particles in an appropriate amount and the coated particles can then be incorporated in a formulation as described above.

The compositions of the present invention can be in any form. These forms include, but are not limited to, solutions, suspensions, dispersions, ointments (including oral ointments), creams, pastes, gels, powders (including tooth powders), toothpastes, lozenges, salve, chewing gum, mouth sprays, pastilles, sachets, mouthwashes, aerosols, tablets, capsules, transdermal patches, that comprise one or more resolvins and/or protectins or their analogues of the invention.

In certain embodiments, an agent targeting the metabolic regulators of the present invention, for example an agent targeting MSP1, MSP2, MPS3, MPS4, MPS5 and/or Ins16, for example a nucleic acid agent or polypeptide agent are administered to a subject as a pharmaceutical composition with a pharmaceutically acceptable carrier. In certain embodiments, these pharmaceutical compositions optionally further comprise one or more additional therapeutic agents. In certain embodiments, the additional therapeutic agent or agents are anti-obesity drugs, drugs for insulin insensitivity, for example insulin, and drugs administered to prevent muscle atrophy and degeneration. Of course, such therapeutic agents are which are known to those of ordinary skill in the art can readily be substituted as this list should not be considered exhaustive or limiting.

In certain embodiments, the endogenous compounds are isolated and/or purified or substantially purified by one or more purification methods described herein or known by those skilled in the art. Generally, the purities are at least 90%, in particular 95% and often greater than 99%. In certain embodiments, the naturally occurring compound is excluded from the general description of the broader genus.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a compound(s) of the present invention within or to the subject such that it can perform its intended function. The term “pharmaceutically acceptable carriers” is intended to include all solvents, diluents, or other liquid vehicle, dispersion or suspension aids, surface active agents, isotonic agents, thickening or emulsifying agents, preservatives, solid binders, lubricants and the like, as suited to the particular dosage form desired. Typically, such compounds are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

In certain embodiments, the compounds of the present invention, for example agents targeting the metabolic regulators of the present invention, for example an agent targeting MSP1, MSP2, MPS3, MPS4, MPS5 and/or Ins16 or functional derivatives thereof, for example a nucleic acid agent or polypeptide agents, may contain one or more acidic functional groups and, thus, are capable of forming pharmaceutically acceptable salts with pharmaceutically acceptable bases. The term “pharmaceutically acceptable salts, esters, amides, and prodrugs as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use of the compounds of the invention. The term “salts” refers to the relatively non-toxic, inorganic and organic acid addition salts of compounds of the present invention.

These salts can be prepared in situ during the final isolation and purification of the compounds or by separately reacting the purified compound in its free base form with a suitable organic or inorganic acid and isolating the salt thus formed. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M., et al., “Pharmaceutical Salts,” J. Pharm. Sci., 1977; 66: 1-19 which is incorporated herein by reference).

The term “pharmaceutically acceptable esters” refers to the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. The term is further intended to include lower hydrocarbon groups capable of being solvated under physiological conditions, e.g., alkyl esters, methyl, ethyl and propyl esters.

As used herein, “pharmaceutically acceptable salts or prodrugs are salts or prodrugs that are, within the scope of sound medical judgment, suitable for use in contact with the tissues of patients without undue toxicity, irritation, allergic response, and the like, commensurate with a reasonable benefit/risk ratio, and effective for their intended use. These compounds include the zwitterionic forms, where possible, of r compounds of the invention.

The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylanunonium, tetraethyl ammonium, methyl amine, dimethyl amine, trimethylamine, triethylamine, ethylamine, and the like (see, e.g., Berge S. M., et al. (1977) J. Pharm. Sci. 66, 1, which is incorporated herein by reference).

The term “prodrug” refers to compounds or agents that are rapidly transformed in vivo to yield the compounds of the invention, for example agents targeting the metabolic regulators of the present invention, for example an agent targeting MSP1, MSP2, MPS3, MPS4, MPS5 and/or Ins16 can be activated by hydrolysis in blood. A thorough discussion is provided in T. Higachi and V. Stella, “Pro-drugs as Novel Delivery Systems,” Vol. 14 of the A. C. S. Symposium Series, and in Bioreversible Carriers in: Drug Design, ed. Edward B. Roche, American Pharmaceutical Association and Pergamon Press, 1987, both of which are hereby incorporated by reference. As used herein, a prodrug is a compound that, upon in vivo administration, is metabolized or otherwise converted to the biologically, pharmaceutically or therapeutically active form of the compound. The prodrug may be designed to alter the metabolic stability or the transport characteristics of a compound, to mask side effects or toxicity, to improve the flavor of a compound or to alter other characteristics or properties of a compound. By virtue of knowledge of pharmacodynamic processes and drug metabolism in vivo, once a pharmaceutically active compound is identified, those of skill in the pharmaceutical art generally can design prodrugs of the compound (see, e.g., Nogrady (1985) Medicinal Chemistry A Biochemical Approach, Oxford University Press, N.Y., pages 388-392). Conventional procedures for the selection and preparation of suitable prodrugs are described, for example, in “Design of Prodrugs,” ed. H. Bundgaard, Elsevier, 1985. Suitable examples of prodrugs include methyl, ethyl and glycerol esters of the corresponding acid.

In other embodiments of the present invention, agents targeting the metabolic regulators of the present invention, for example an agent targeting MSP1, MSP2, MPS3, MPS4, MPS5 and/or Ins16 or functional derivatives thereof, can be conjugated or covalently attached to another targeting agent to increase their tissue specificity and targeting to a cell, for example a muscle cells. Targeting agents can include, for example without limitation, antibodies, cytokines and receptor ligands, as discussed in the section entitled “targeting.” In some embodiments, the targeting agent is overexpressed on the cells to be targeted, for example the muscle cells as compared to non-muscle cells.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfate, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for intravenous, oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar—agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients. In one aspect, a solution of resolvin and/or protectin or precursor or analog thereof can be administered as eye drops for ocular neovascularization or ear drops to treat otitis.

Liquid dosage forms for oral administration of the compounds of the invention include pharmaceutically acceptable emulsions, microemulsions, solutions, suspensions, syrups and elixirs.

In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some instances, agents targeting the metabolic regulators of the present invention, for example agents targeting at least one of MSP1, MSP2, MPS3, MPS4, MPS5 and/or Ins16 or functional derivatives thereof, may be in a formulation suitable for rectal or vaginal administration, for example as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore release the active compound. Suitable carriers and formulations for such administration are known in the art.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of the compounds (resolvins and/or protectins and/or precursors or analogues thereof) of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of ordinary skill in the art.

Remington's Pharmaceutical sciences Ed. Germany, Merk Publishing, Easton, Pa., 1995 (the contents of which are hereby incorporated by reference), discloses various carriers used in formulating pharmaceutical compositions and known techniques for the preparation thereof. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; malt; gelatin; talc; excipients such as cocoa butter and: suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; water; isotonic saline; Ringer's solution, ethyl alcohol, and phosphate buffer solutions, as well as other non-toxic compatible lubricants such as sodium lauryl sulfate and magnesium sulfate, as well as coloring agents, releasing agents, coating agents, sweetening, flavoring and perfuming agents, preservatives and antioxidants can also be present in the composition, according to the judgment of the formulator.

### Other Uses of the Genes of the Present Invention

The present invention also provides methods for using the metabolic regulators of the present invention, for example MSP1, MSP2, MPS3, MPS4, MPS5 and/or Ins16 to identify other factors associated with muscle growth, obesity, insulin sensitivity, muscle mass, fat mass, muscle growth and cardiovascular function, including identification of mRNAs, functional RNAs, e.g., microRNAs. Such methods are known to the skilled artisan and may include methods described below.

The present invention also provides methods for using the metabolic regulators of the present invention, for example MSP1, MSP2, MPS3, MPS4, MPS5 and/or Ins16 to in assays to identify agents that selectively activate or inhibit the metabolic factors, for example to identify agents to be used by the methods of the present invention for the treatment of disorders and conditions associated with muscle growth, obesity, insulin sensitivity and cardiovascular function.

The metabolic regulators of the present invention, for example MSP1, MSP2, MPS3, MPS4, MPS5 and/or Ins16 or homologues thereof are useful for further characterization in order to examine the effects of the identified genes and gene products on muscle growth and biology, as well as angiogenesis, insulin sensitivity and fat mass reduction/growth.

The metabolic regulators of the present invention, for example MSP1, MSP2, MPS3, MPS4, MPS5 and/or Ins16 may be used for the construction of transgenic animals, e.g., knock-out animals, e.g., animals with exogenous expression. Furthermore, transgenic animals, including knock-out animals, may be used as animal models of diseases and disorders, for example obesity models, muscular dystrophy mouse models, AIDs and AIDs-related animals models, glucose insensitivity and insulin insensitivity, for example by using the methods as disclosed in the Examples, or breeding such mice with transgenic mouse models of such diseases.

For example, such transgenic animals for at least one metabolic regulator of the present invention, for example at least one of MSP1, MSP2, MPS3, MPS4, MPS5 and/or Ins16, for example overexpressing, knock-in and/or knock-out at least one metabolic regulator, may be bred into animals of varying genetic backgrounds, including animals with phenotypes of interest, e.g., obesity, diabetes, angiogenic defects, cardiovascular defects. Such animals are known to the skilled artisan, and, for example, can be found in the Mouse Genome Database (Blake J A, Richardson J E, Bult C J, Kadin J A, Eppig J T, and the members of the Mouse Genome Database Group. 2003. MGD: The Mouse Genome Database. Nucleic Acids Res 31: 193-195; Eppig J T., Blake, J A, Burkhart D L, Goldsmith C W, Lutz C M, Smith C L. 2002. Corralling conditional mutations: a unified resource for mouse phenotypes. Genesis 32:63-65) or the Oak Ridge National Laboratory mutant mouse database.

In alternative embodiments, cells and/or transgenic animals of at least one metabolic regulator, for example animals overexpressing, knock-in and/or knock-out at least one of MSP1, MSP2, MPS3, MPS4, MPS5 and/or Ins16 are used in an assay to identify proteins that affect muscle growth, angiogenesis, obesity, insulin sensitivity and/or cardiovascular function.

The metabolic regulators of the present invention, for example MSP1, MSP2, MPS3, MPS4, MPS5 and/or Ins16 may be analyzed computationally or experimentally for characteristics of interest. In one embodiment, the genes identified as associated with metabolic regulators of the present invention, for genes associated with MSP1, MSP2, MPS3, MPS4, MPS5 and/or Ins16 are computationally screened for characteristics identifying the genes as secreted factors, i.e., possession of putative signal sequences and lack of putative transmembrane domains. Any other domain or sequence characteristics of interest, e.g., nucleic acid or amino acid sequence, may be utilized in designing appropriate agents for therapeutic use to target at least one metabolic regulators of the present invention, for example at least one of MSP1, MSP2, MPS3, MPS4, MPS5 and/or Ins16.

Transgenic animals for metabolic regulators, for example animals overexpressing, knock-in and/or knock-out for MSP1, MSP2, MPS3, MPS4, MPS5 and/or Ins16 can also be used in assays to test agents for efficacy on muscle development, muscle growth, obesity, insulin sensitivity and cardiovascular function. In some embodiments the assay is performed on test animals, or transgenic animals for the metabolic regulators or samples or specimens (e.g., a biopsy) from such transgenic animals. In some cases, it will be advantageous to measure the markers of muscle growth, obesity, insulin sensitivity and cardiovascular function in samples, blood, which may be obtained from the test animal without sacrifice of the animal.

A transgenic animal, for example a transgenic non-human animal can be produced which contains selected systems that allow for regulated expression of the metabolic regulator of the present invention, for example a Cre/Lox inducible expression system can be used, which is known by persons of ordinary skill in the art.

A transgenic animal, e.g., an invertebrate, such as drosophila, e.g., a vertebrate, such as a mammal, such as a rodent, such as a mouse or a rat, is an animal having cells that contain a transgene, which transgene was introduced into the animal or an ancestor of the animal at a prenatal, e.g., an embryonic stage. A transgene is a DNA, e.g., a metabolic regulator of the present invention, for example MSP1, MSP2, MPS3, MPS4, MPS5 and/or Ins16 or homologue or variant thereof, which is integrated into the nuclear genome of a cell from which a transgenic animal develops. The transgene may be integrated into all cells in the animal, including incorporation into the germline of the animal. Alternatively, the animal may be chimeric for the transgene. Methods for generating transgenic animals, particularly animals such as mice or rats, have become conventional in the art and are described, for example, in Ausubel et al. (eds) “Current Protocols in Molecular Biology” John Wiley & Sons, Inc., in Hogan, B., Manipulating the Mouse Embryo, (Cold Spring Harbor Laboratory Press, Cold Spring Harbor, N.Y., 1986) and in U.S. Pat. Nos. 5,614,396 5,487,992, 5,464,764, 5,387,742, 5,347,075, 5,298,422, 5,288,846, 5,221,778, 5,175,384, 5,175,383, 4,873,191, 4,870,009, 4,736,866 as well as Burke and Olson, Methods in Enzymology, 194:251-270, 1991; Capecchi, Science 244:1288-1292, 1989; Davies et al., Nucleic Acids Research, 20 (11) 2693-2698, 1992; Dickinson et al., Human Molecular Genetics, 2(8):1299-1302, 1993; Duff and Lincoln, “Insertion of a pathogenic mutation into a yeast artificial chromosome containing the human APP gene and expression in ES cells”, Research Advances in Alzheimer's Disease and Related Disorders, 1995; Huxley et al., Genomics, 9:742-750 1991; Jakobovits et al., Nature, 362:255-261 1993; Lamb et al., Nature Genetics, 5: 22-29, 1993; Pearson and Choi, Proc. Natl. Acad. Sci. USA, 1993, 90:10578-82; Rothstein, Methods in Enzymology, 194:281-301, 1991; Schedl et al., Nature, 362: 258-261, 1993; Strauss et al., Science, 259:1904-1907, 1993, WO 94/23049, WO93/14200, WO 94/06908 and WO 94/28123 also provide information.

The present invention is not limited to a particular animal. A variety of human and non-human animals are contemplated. For example, in some embodiments, rodents (e.g., mice or rats) or primates are provided as animal models for alterations in fat metabolism and screening of compounds.

In other embodiments, the present invention provides commercially useful transgenic animals (e.g., livestock animals such as pigs, cows, or sheep) that are transgenic for the metabolic regulators of the present invention, for example MSP1, MSP2, MPS3, MPS4, MPS5 and/or Ins16, i.e for example animals overexpressing, knock-in and/or knock-out for at least one metabolic regulator of the present invention. It is contemplated that meat from such animals will have desirable properties such as lower fat content and higher muscle content. Any suitable technique for generating transgenic livestock may be utilized. In some preferred embodiments, retroviral vector infection is utilized (See e.g., U.S. Pat. No. 6,080,912 and WO/0030437; each of which is herein incorporated by reference in its entirety).

Similar methods are used for production of other transgenic animals. A transgenic founder animal can be identified based upon the presence of the transgene in its genome and/or expression of transgenic mRNA in tissues or cells of the animals. A transgenic founder animal can then be used to breed additional animals carrying the transgene. Moreover, transgenic animals carrying a transgene can further be bred to other transgenic animals carrying other transgenes.

Any techniques known in the art may be used to introduce nucleic acids encoding a metabolic regulators of the present invention, for a nucleic acid encoding for example MSP1, MSP2, MPS3, MPS4, MPS5 and/or Ins16 or fragments or homologues thereof, expressible into animals to produce the mammal lines of animals. Such techniques include, but are not limited to, pronuclear microinjection (U.S. Pat. No. 4,873,191); retrovirus mediated gene transfer into germ lines [Van der Putten, et al., 1985, Proc. Natl. Acad. Sci., USA 82, 6148-6152]; gene targeting in embryonic stem cells, such as homologous recombination mediated gene targeting [Thompson, et al., 1989, Cell 56, 313-321 and U.S. Pat. No. 5,614,396]; electroporation of embryos [Lo, 1983, Mol. Cell. Biol. 3, 1803-1814]; and sperm-mediated gene transfer [Nakanishi and Iritani, Mol. Reprod. Dev. 36:258-261 (1993); Maione, Mol. Reprod. Dev. 59:406 (1998); Lavitrano et al. Transplant. Proc. 29:3508-3509 (1997); Lavitrano et al., Proc. Natl. Acad. Sci. USA 99:14230-5 (2002); Lavitrano et al., Mol. Reprod. Dev. 64-284-91 (2003)). Similar techniques are also described in U.S. Pat. No. 6,376,743; U.S. Pat. Publ. Nos. 20010044937, 20020108132, and 20050229263. Other methods of preparing transgenic animals are disclosed, for example, in U.S. Pat. No. 5,633,076 or 6,080,912; and in International Patent Publications WO 97/47739, WO 99/37143, WO 00/75300, WO 00/56932, and WO 00/08132, the disclosures of which are incorporated herein by reference in their entirety.

Of further interest is the examination of protein expression in cells and/or transgenic animals expressing the metabolic regulators of the present invention, for example cells and/or transgenic animals expressing MSP1, MSP2, MPS3, MPS4, MPS5 and/or Ins16 for example, such cells and/or transgenic mice are useful for examination of the proteins expressed in the cells on induction (transiently or constitutively) of the metabolic regulator, to analyze their effect on angiogenesis, glucose sensitivity, fat mass and hypertrophy and muscle regeneration. Analysis of protein expression of tissues and cells of non-transgenic cells and/or animals as compared with animals and/or transgenic cells transgenic for the metabolic regulators of the present invention, for example MSP1, MSP2, MPS3, MPS4, MPS5 and/or Ins16. Such methods are useful to identify other metabolic regulators and are also encompassed for use as metabolic regulators in the present invention.

Method for Screening for an Agent that Modulates a Factor Associated with Muscle Growth

The present invention provides for methods to screen for agents that modulate the metabolic regulators of the present invention, for example an agent that modulates MSP1, MSP2, MPS3, MPS4, MPS5 and/or Ins16 or their homologues. Transgenic animal models and/or cells expressing a metabolic regulators can also be used to assay test compounds (e.g., a drug candidate) for efficacy on muscle development, muscle growth, obesity, insulin sensitivity and cardiovascular function in test animals, or in samples or specimens (e.g., a biopsy) from the test animals. In some cases, it will be advantageous to measure markers of muscle growth, obesity, insulin sensitivity and cardiovascular function in samples, for example blood, which may be obtained from the test animal without sacrifice of the animal.

Test Compounds

The term “agent” or “compound” as used herein and throughout the specification means any organic or inorganic molecule, including modified and unmodified nucleic acids such as antisense nucleic acids, RNAi, such as siRNA or shRNA, peptides, peptidomimetics, receptors, ligands, and antibodies.

In the methods of the present invention, a variety of test agents and physical conditions from various sources can be screened for the ability of the agent to alter the activity and/or expression of the metabolic regulators of the present invention, for example agent that modulate MSP1, MSP2, MPS3, MPS4, MPS5 and/or Ins16.

Generally, the effect of an agent on a cell or transgenic cell and/or animal for the metabolic regulators animal is compared with comparative cell or animal in the absence of test compound(s). In cases where the animal is sacrificed, a baseline can be established based on an average or a typical value from a control animal(s) that have not received the administration of any test agent. Once such a baseline is determined, test agents can be administered to additional cells or test animals, where deviation from the baseline indicates that the test agent had an effect on the activity and/or expression of the metabolic regulator of the present invention.

The test agents can be any molecule, agent, or other substance which can be administered to a cell or test animal. In some cases, the agent does not substantially interfere with the activity of a metabolic regulator in a cell or animal. Suitable test agents may be small molecules, biological polymers, such as polypeptides, polysaccharides, polynucleotides, and the like. The test agents will typically be administered to the animal at a dosage of from 1 ng/kg to 10 mg/kg, usually from 10 μg/kg to 1 mg/kg. Test agents can be identified that are useful as in the therapeutic methods of the present invention as agents that have a desired modulation of the expression and/or activity of a metabolic regulator, for example have a desired effect on at least one of MSP1, MSP2, MPS3, MPS4, MPS5 and/or Ins16.

In some embodiments, test agents can be from diversity libraries, such as random or combinatorial peptide or non-peptide libraries. Many libraries are known in the art, such as, for example, chemically synthesized libraries, recombinant phage display libraries, and in vitro translation-based libraries.

Examples of chemically synthesized libraries are described in Fodor et al. (Science 251:767-73 (1991)), Houghten et al. (Nature 354:84-86 (1991)), Lam et al. (Nature 354:82-84 (1991)), Medynski (Bio/Technology 12:709-10 (1994)), Gallop et al. (J. Med. Chem. 37:1233-51 (1994)), Ohlmeyer et al. (Proc. Natl. Acad. Sci. USA 90:10922-26 (1993)), Erb et al. (Proc. Natl. Acad. Sci. USA 91:11422-26 (1994)), Houghten et al. (Biotechniques 13:412-21 (1992)), Jayawickreme et al. (Proc. Natl. Acad. Sci. USA 91:1614-18 (1994)), Salmon et al. (Proc. Natl. Acad. Sci. USA 90:11708-12 (1993)), International Patent Publication WO 93/20242, and Brenner and Lerner (Proc. Natl. Acad. Sci. USA 89:5381-83 (1992)).

Examples of phage display libraries are described in Scott and Smith (Science 249:386-90 (1990)), Devlin et al. (Science 249:404-06 (1990)), Christian et al. (J. Mol. Biol. 227:711-18 (1992)), Lenstra (J. Immunol. Meth. 152:149-57 (1992)), Kay et al. (Gene 128:59-65 (1993)), and International Patent Publication WO 94/18318.

In vitro translation-based libraries include, but are not limited to, those described in International Patent Publication WO 91/05058, and Mattheakis et al. (Proc. Natl. Acad. Sci. USA 91:9022-26 (1994)). By way of examples of nonpeptide libraries, a benzodiazepine library (see, e.g., Bunin et al., Proc. Natl. Acad. Sci. USA 91:4708-12 (1994)) can be adapted for use. Peptide libraries (see, e.g., Simon et al., Proc. Natl. Acad. Sci. USA 89:9367-71 (1992)) can also be used. Another example of a library that can be used, in which the amide functionalities in peptides have been permethylated to generate a chemically transformed combinatorial library, is described by Ostresh et al. (Proc. Natl. Acad. Sci. USA 91:11138-42 (1994)).

The following examples are provided merely as illustrative of various aspects of the invention and shall not be construed to limit the invention in any way.

Agents to be screened can be naturally occurring or synthetic molecules. Agents to be screened can also be obtained from natural sources, such as; marine microorganisms, algae, plants, and fungi. The test agents can also be minerals or oligo agents. Alternatively, test agents can be obtained from combinatorial libraries of agents, including peptides or small molecules, or from existing repertories of chemical agents synthesized in industry, e.g., by the chemical, pharmaceutical, environmental, agricultural, marine, cosmetic, drug, and biotechnological industries. Test agents can include, e.g., pharmaceuticals, therapeutics, agricultural or industrial agents, environmental pollutants, cosmetics, drugs, organic and inorganic agents, lipids, glucocorticoids, antibiotics, peptides, proteins, sugars, carbohydrates, chimeric molecules, and combinations thereof.

Combinatorial libraries can be produced for many types of agents that can be synthesized in a step-by-step fashion. Such agents include polypeptides, proteins, nucleic acids, beta-turn mimetics, polysaccharides, phospholipids, hormones, prostaglandins, steroids, aromatic agents, heterocyclic agents, benzodiazepines, oligomeric N-substituted glycines and oligocarbamates. In the method of the present invention, the preferred test agent is a small molecule, nucleic acid and modified nucleic acids, peptide, peptidomimetic, protein, glycoprotein, carbohydrate, lipid, or glycolipid. Preferably, the nucleic acid is DNA or RNA.

Large combinatorial libraries of agents can be constructed by the encoded synthetic libraries (ESL) method described in Affymax, WO 95/12608, Affymax WO 93/06121, Columbia University, WO 94/08051, Pharmacopeia, WO 95/35503 and Scripps, WO 95/30642 (each of which is incorporated herein by reference in its entirety for all purposes). Peptide libraries can also be generated by phage display methods. See, e.g., Devlin, WO 91/18980. Agents to be screened can also be obtained from governmental or private sources, including, e.g., the DIVERSet E library (16,320 agents) from ChemBridge Corporation (San Diego, Calif.), the National Cancer Institute's (NCI) Natural Product Repository, Bethesda, Md., the NCI Open Synthetic Compound Collection, Bethesda, Md., NCI's Developmental Therapeutics Program, or the like.

Additionally, natural and synthetically produced libraries and agents are readily modified through conventional chemical, physical, and biochemical means. In addition, known pharmacological agents may be subject to directed or random chemical modifications, such as acylation, alkylation, esterification, amidification, etc.

The agent formulations may conveniently be presented in unit dosage form, e.g., tablets and sustained release capsules, and in liposomes, and may be prepared by any methods well know in the art of pharmacy. (See, for example, Remington: The Science and Practice of Pharmacy by Alfonso R. Gennaro (Ed.) 20th edition, Dec. 15, 2000, Lippincott, Williams & Wilkins; ISBN: 0683306472.).

Screening agents for potential effectiveness in modulating transcription and/or protein expression of metabolic regulators of the present invention, for example agent that modulate MSP1, MSP2, MPS3, MPS4, MPS5 and/or Ins16, be accomplished by a variety of means well known by a person skilled in the art.

To screen the agents described above for ability to modulate transcription and/or expression of metabolic regulators of the present invention, for example, MSP2, MPS3, MPS4, MPS5 and/or Ins16, the test agents should be administered to the test subject. In one embodiment the test subject is a culture of cells comprised of cells derived from muscle, e.g., skeletal muscle. The cells derived from muscle may be a primary cell culture or an immortalized cell line from a normal or a tumorous muscle. In another embodiment, the test subject is an animal with muscle, e.g., skeletal muscle. The animal with muscle can be, but is not limited to, a fruit fly, a frog, a rodent such as a mouse or a rat, a rabbit, a non-human primate, and a human. The muscle derived cells can be obtained from the muscle of a an animal, including but not limited to, fruit fly, a frog, a rodent such as a mouse or a rat, a rabbit, a non-human primate and a human.

The test agents can be administered, for example, by diluting the agents into the medium wherein the cell is maintained, mixing the test agents with the food or liquid of the animal with muscle, topically administering the agent in a pharmaceutically acceptable carrier on the animal with muscle, using three-dimensional substrates soaked with the test agent such as slow release beads and the like and embedding such substrates into the animal, intramuscularly administering the agent, parenterally administering the agent.

A variety of other reagents may also be included in the mixture. These include reagents such as salts, buffers, neutral proteins, e.g. albumin, detergents, etc. which may be used to facilitate optimal protein-protein and/or protein-nucleic acid binding and/or reduce non-specific or background interactions, etc. Also, reagents that otherwise improve the efficiency of the assay, such as protease inhibitors, nuclease inhibitors, antimicrobial agents, etc. may be used.

The language “pharmaceutically acceptable carrier” is intended to include substances capable of being co-administered with the agent and which allows the active ingredient to perform its intended function of preventing, ameliorating, arresting, or eliminating a disease(s) of the nervous system. Examples of such carriers include solvents, dispersion media, adjuvants, delay agents and the like. The use of such media and agents for pharmaceutically active substances is well known in the art. Any conventional media and agent compatible with the agent may be used within this invention.

The agents can be formulated according to the selected route of administration. The addition of gelatin, flavoring agents, or coating material can be used for oral applications. For solutions or emulsions in general, carriers may include aqueous or alcoholic/aqueous solutions, emulsions or suspensions, including saline and buffered media. Parenteral vehicles can include sodium chloride, potassium chloride among others. In addition intravenous vehicles can include fluid and nutrient replenishers, electrolyte replenishers among others.

Preservatives and other additives can also be present. For example, antimicrobial, antioxidant, chelating agents, and inert gases can be added (see, generally, Remington's Pharmaceutical Sciences, 16th Edition, Mack, 1980).

Screening for an agent that causes an increase in transcription or protein expression of a metabolic regulator of the present invention, for example agent that modulates at least one of MSP1, MSP2, MPS3, MPS4, MPS5 and/or Ins16 can be accomplished using measurements of gene transcription and/or measurements of protein expression of the metabolic regulator of interest. Measurements of gene transcription can include direct measurements of gene transcription of the factor associated with muscle growth or measurements of a reporter gene. Similarly, measurements of protein expression can include measurements of protein expression of the factor associated with muscle growth or measurements of a reporter gene.

As noted above, screening assays are generally carried out in vitro, for example, in cultured cells, in a biological sample, e.g., muscle, e.g., skeletal muscle, or fractions thereof. For ease of description, cell cultures, biological samples, and fractions are referred to as “samples” below. The sample is generally derived from an animal (e.g., any of the research animals mentioned above), preferably a mammal, and more preferably from a human.

The reporter gene assay (Tamura, et al., Transcription Factor Research Method, Yodosha, 1993) is a method for assaying the regulation of gene expression using as the marker the expression of a reporter gene.

Detection and quantification gene expression of the factor associated with muscle growth may be carried out through any of the methods described above in connection with identification of factors associated with muscle growth. Any gene transcription and polypeptide or protein expression assays known to the skilled artisan can be used to detect either the transcription and/or expression of the factor associated with muscle growth. Alternatively, when a reporter gene is utilized, the transcription and/or expression of the reporter gene may also be detected in place of the factor associated with muscle growth utilizing the amplification based, hybridization based and/or polypeptide based assays.

Suitable amplification based methods include, but are not limited to, polymerase chain reaction (PCR); reverse-transcription PCR(RT-PCR); ligase chain reaction (LCR) (see Wu and Wallace (1989) Genomics 4: 560, Landegren et al. (1988) Science 241: 1077, and Barringer et al. (1990) Gene 89: 117; transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874); dot PCR, and linker adapter PCR, etc.

Methods of detecting and/or quantifying polynucleotides using nucleic acid hybridization techniques, e.g., Northern Blots, are known to those of skill in the art (see Sambrook et Molecular Cloning: A Laboratory Manual, 2d Ed. vol. 1-3, Cold Spring Harbor Press, NY, 1989). Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587. Methods of optimizing hybridization conditions are described, e.g., in Tijssen (1993) Laboratory Techniques in Biochemistry and Molecular Biology, Vol. 24: Hybridization With Nucleic Acid Probes, Elsevier, N.Y.).

Polypeptides of metabolic regulatorscan be detected and quantified by any of a number of methods well known to those of skill in the art. Examples of analytic biochemical methods suitable for detecting protein include electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunohistochemistry, affinity chromatography, immunoelectrophoresis, radioimmunoassay (RIA), enzyme-linked immunosorbent assays (ELISAs), immunofluorescent assays, Western blotting, and the like.

Antibodies to the factor associated with muscle growth (preferably anti-mammalian; more preferably anti-human) may be produced by methods well known to those skilled in the art. Fragments of antibodies to the factor associated with muscle growth may be produced by cleavage of the antibodies in accordance with methods well known in the art. For example, immunologically active F(ab′) and F(ab′)2 fragments may be generated by treating the antibodies with an enzyme such as pepsin.

### Binding Partner Identification

One method for identifying agents that modulate the metabolic regulator of the present invention is to identify binding partners to the metabolic regulator. Identification of such proteins that bind and modulate the activity of metabolic regulators of the present invention, for example binding partners of MSP1, MSP2, MPS3, MPS4, MPS5 and/or Ins16 includes, for example, providing a library and selecting from the library one or more members that encode a protein that binds to the metabolic regulator antigen. The selection can be performed in a number of ways. For example, the library can be a display library. The metabolic regulator can be tagged and recombinantly expressed. The metabolic regulator is purified and attached to a support, e.g., to affinity beads, or paramagnetic beads or other magnetically responsive particles. The metabolic regulator can also be expressed on the surface of a cell. Members of the display library that specifically bind to the cell can be selected.

In one embodiment, a display library is used to identify proteins that bind to a metabolic regulator of the present invention, for example proteins that bind to MSP1, MSP2, MPS3, MPS4, MPS5 and/or Ins16. A display library is a collection of entities; each entity includes an accessible protein component and a recoverable component (e.g., a nucleic acid) that encodes or identifies the protein component. The protein component can be of any length, e.g. from three amino acids to over 300 amino acids. In a selection, the protein component of each member of the library is probed with a metabolic regulator protein and if the protein component binds to the metabolic regulator, the display library member is identified, e.g., by retention on a support. The display libraries can be constructed from cDNAs derived from tissue derived from the transgenic animals of the present invention wherein the accessible protein components are encoded by the cDNAs. The cDNAs contained in the display library may be the result of cDNA subtractions or other cDNA identification procedures outlined above. Thus, the cDNAs may be the result of comparing induced relative to non-induced transgenics or the result of comparing induced transgenics of varying genetic backgrounds or any other comparison of gene expression relating to the methods of the present invention.

Retained display library members are recovered from the support and analyzed. The analysis can include amplification and a subsequent selection under similar or dissimilar conditions. For example, positive and negative selections can be alternated. The analysis also can include determining the amino acid sequence of the protein component and purification of the protein component for detailed characterization.

A variety of formats can be used for display libraries. Examples include the following.

Phage Display. One format utilizes viruses, particularly bacteriophages. This format is termed “phage display.” The protein component is typically covalently linked to a bacteriophage coat protein. The linkage results form translation of a nucleic acid encoding the protein component fused to the coat protein. The linkage can include a flexible peptide linker, a protease site, or an amino acid incorporated as a result of suppression of a stop codon. Phage display is described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem. 274:18218-30; Hoogenboom et al. (1998) Immunotechnology 4:1-20; Hoogenboom et al. (2000) Immunol Today 2:371-8; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Rebar et al. (1996) Methods Enzymol. 267:129-49; Hoogenboom et al. (1991) Nuc Acid Res 19:4133-4137; and Barbas et al. (1991) PNAS 88:7978-7982.

Phage display systems have been developed for filamentous phage (phage fl, fd, and M13) as well as other bacteriophage (e.g., T7 bacteriophage and lambdoid phages; see, e.g., Santini (1998) J. Mol. Biol. 282:125-135; Rosenberg et al. (1996) Innovations 6:1-6; Houshm et al. (1999) Anal Biochem 268:363-370). The filamentous phage display systems typically use fusions to a minor coat protein, such as gene III protein, and gene VIII protein, a major coat protein, but fusions to other coat proteins such as gene VI protein, gene VII protein, gene IX protein, or domains thereof also can be used (see, e.g., WO 00/71694). In one embodiment, the fusion is to a domain of the gene III protein, e.g., the anchor domain or “stump,” (see, e.g., U.S. Pat. No. 5,658,727 for a description of the gene III protein anchor domain). It also is possible to physically associate the protein being displayed to the coat using a non-peptide linkage, e.g., a non-covalent bond or a non-peptide covalent bond. For example, a disulfide bond and/or c-fos and c-jun coiled-coils can be used for physical associations (see, e.g., Crameri et al. (1993) Gene 137:69 and WO 01/05950).

Bacteriophage displaying the protein component can be grown and harvested using standard phage preparatory methods, e.g. PEG precipitation from growth media. After selection of individual display phages, the nucleic acid encoding the selected protein components, by infecting cells using the selected phages. Individual colonies or plaques can be picked, the nucleic acid isolated and sequenced.

Cell-based Display. In still another format the library is a cell-display library. Proteins are displayed on the surface of a cell, e.g., a eukaryotic or prokaryotic cell. Exemplary prokaryotic cells include E. coli cells, B. subtilis cells, and spores (see, e.g., Lu et al. (1995) Biotechnology 13:366). Exemplary eukaryotic cells include yeast (e.g., Saccharomyces cerevisiae, Schizosaccharomyces pombe, Hanseula, or Pichia pastoris). Yeast surface display is described, e.g., in Boder and Wittrup (1997) Nat. Biotechnol. 15:553-557 and WO 03/029456, which describes a yeast display system that can be used to display immunoglobulin proteins such as Fab fragments and the use of mating to generate combinations of heavy and light chains.

In one embodiment, diverse nucleic acid sequences are cloned into a vector for yeast display. The cloning joins the variegated sequence with a domain (or complete) yeast cell surface protein, e.g., Aga2, Aga1, Flo1, or Gas 1. A domain of these proteins can anchor the polypeptide encoded by the variegated nucleic acid sequence by a transmembrane domain (e.g., Flo1) or by covalent linkage to the phospholipid bilayer (e.g., Gas1). The vector can be configured to express two polypeptide chains on the cell surface such that one of the chains is linked to the yeast cell surface protein. For example, the two chains can be immunoglobulin chains.

Ribosome Display. RNA and the polypeptide encoded by the RNA can be physically associated by stabilizing ribosomes that are translating the RNA and have the nascent polypeptide still attached. Typically, high divalent Mg²⁺ concentrations and low temperature are used. See, e.g., Mattheakis et al. (1994) Proc. Natl. Acad. Sci. USA 91:9022 and Hanes et al. (2000) Nat. Biotechnol. 18:1287-92; Hanes et al. (2000) Methods Enzymol. 328:404-30; and Schaffitzel et al. (1999) J Immunol Methods. 231(1-2):119-35.

Polypeptide-Nucleic Acid Fusions. Another format utilizes polypeptide-nucleic acid fusions. Polypeptide-nucleic acid fusions can be generated by the in vitro translation of mRNA that include a covalently attached puromycin group, e.g., as described in Roberts and Szostak (1997) Proc. Natl. Acad. Sci. USA 94:12297-12302, and U.S. Pat. No. 6,207,446. The mRNA can then be reverse transcribed into DNA and crosslinked to the polypeptide.

Other Display Formats. Yet another display format is a non-biological display in which the protein component is attached to a non-nucleic acid tag that identifies the polypeptide. For example, the tag can be a chemical tag attached to a bead that displays the polypeptide or a radiofrequency tag (see, e.g., U.S. Pat. No. 5,874,214).

ELISA. Proteins encoded by a display library can also be screened for a binding property using an ELISA assay. For example, each protein is contacted to a microtitre plate whose bottom surface has been coated with the target, e.g., a limiting amount of the target. The plate is washed with buffer to remove non-specifically bound polypeptides. Then the amount of the protein bound to the plate is determined by probing the plate with an antibody that can recognize the polypeptide, e.g., a tag or constant portion of the polypeptide. The antibody is linked to an enzyme such as alkaline phosphatase, which produces a colorimetric product when appropriate substrates are provided. The protein can be purified from cells or assayed in a display library format, e.g., as a fusion to a filamentous bacteriophage coat. Alternatively, cells (e.g., live or fixed) that express the target molecule, e.g., a metabolic regulator Akt1, e.g., constitutively active isoform of Akt1, can be plated in a microtitre plate and used to test the affinity of the peptides/antibodies present in the display library or obtained by selection from the display library.

In another version of the ELISA assay, each polypeptide of a diversity strand library is used to coat a different well of a microtitre plate. The ELISA then proceeds using a constant target molecule to query each well.

Homogeneous Binding Assays. The binding interaction of candidate protein with a target can be analyzed using a homogenous assay, i.e., after all components of the assay are added, additional fluid manipulations are not required. For example, fluorescence resonance energy transfer (FRET) can be used as a homogenous assay (see, for example, Lakowicz et al., U.S. Pat. No. 5,631,169; Stavrianopoulos, et al., U.S. Pat. No. 4,868,103). A fluorophore label on the first molecule (e.g., the molecule identified in the fraction) is selected such that its emitted fluorescent energy can be absorbed by a fluorescent label on a second molecule (e.g., the target) if the second molecule is in proximity to the first molecule. The fluorescent label on the second molecule fluoresces when it absorbs to the transferred energy. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A binding event that is configured for monitoring by FRET can be conveniently measured through standard fluorometric detection means well known in the art (e.g., using a fluorimeter). By titrating the amount of the first or second binding molecule, a binding curve can be generated to estimate the equilibrium binding constant.

Another example of a homogenous assay is Alpha Screen (Packard Bioscience, Meriden Conn.). Alpha Screen uses two labeled beads. One bead generates singlet oxygen when excited by a laser. The other bead generates a light signal when singlet oxygen diffuses from the first bead and collides with it. The signal is only generated when the two beads are in proximity. One bead can be attached to the display library member, the other to the target. Signals are measured to determine the extent of binding.

The homogenous assays can be performed while the candidate protein is attached to the display library vehicle, e.g., a bacteriophage or using a candidate protein as free molecule.

Surface Plasmon Resonance (SPR). The binding interaction of a molecule isolated from a display library and a target can be analyzed using SPR. SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden).

Information from SPR can be used to provide an accurate and quantitative measure of the equilibrium dissociation constant (Kd), and kinetic parameters, including Kₒₙ and K_{off}, for the binding of a biomolecule to a target. Such data can be used to compare different biomolecules. For example, proteins encoded by nucleic acid selected from a library of diversity strands can be compared to identify individuals that have high affinity for the target or that have a slow K_{off}. This information can also be used to develop structure-activity relationships (SAR). For example, the kinetic and equilibrium binding parameters of matured versions of a parent protein can be compared to the parameters of the parent protein. Variant amino acids at given positions can be identified that correlate with particular binding parameters, e.g., high affinity and slow K_{off}. This information can be combined with structural modeling (e.g., using homology modeling, energy minimization, or structure determination by crystallography or NMR). As a result, an understanding of the physical interaction between the protein and its target can be formulated and used to guide other design processes.

Protein Arrays. Polypeptides identified from the display library can be immobilized on a solid support, for example, on a bead or an array. For a protein array, each of the polypeptides is immobilized at a unique address on a support. Typically, the address is a two-dimensional address. See, for example, MacBeath et al., 2000, Science, 289:1760-1763 and Bertone et al. 2005, FEBS Journal, 272:5400-5411.

Cellular Assays. Candidate polypeptides can be selected from a library by transforming the library into a host cell; the library could have been previously identified from a display library. For example, the library can include vector nucleic acid sequences that include segments that encode the polypeptides and that direct expression, e.g., such that the polypeptides are produced within the cell, secreted from the cell, or attached to the cell surface. The cells can be screened or selected for polypeptides that bind to the Akt1, e.g., as detected by a change in a cellular phenotype or a cell-mediated activity. For example, in the case of an antibody that binds to Akt1, the activity may be an in vitro assay for cell invasion. In one embodiment, the antibody is contacted to an invasive mammalian cell, e.g., a carcinoma cell, e.g., JEG-3 (choriocarcinoma) cell. The ability of the cell to invade a matrix is evaluated. The matrix can be an artificial matrix, e.g., Matrigel, gelatin, etc., or a natural matrix, e.g., extracellular matrix of a tissue sample, or a combination thereof. For example, the matrix can be produced in vitro by a layer of cells.

### EXAMPLES

The examples presented herein relate to the use of MSP3, MP5 and/or Ins16 and derivatives thereof for the treatment of disease and or agonists or antagonists thereof, for the treatment of diseases and disorders associated with angiogenesis, muscle degeneration, muscle atrophy, obesity, glucose intolerance and/or insulin insensitivity. MSP3, MP5 and/or Ins16 and derivatives thereof, or agonists and/or antagonists can be used alone, together in any combination and in conjunction with other therapeutic agents. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

Methods

Skeletal muscle-specific conditional Akt1 TG mice. MCK-rtTA TG mice (Grill et al., 2003) were crossed with Tet-myrAkt1 TG mice (Shiojima et al., 2005) to generate DTG mice. For Akt1 transgene expression, DTG mice were treated with DOX (0.5 mg/ml) in drinking water, and DOX water was removed to repress the transgene expression. MCK-rtTA single TG littermates were used as controls and treated with DOX in the same manner as DTG mice.

Animal care and diet treatments. Study protocols were approved by the Institutional Animal Care and Use Committee at Boston University. Mice were housed at 24° C. on a fixed 12-h light/dark cycle. Mice were fed either a normal chow diet or a high-fat/sucrose diet (HF diet: Diet No. F1850, BIO-SERV) (Harte et al., 1999) as indicated. Food consumption and body weight was monitored daily in individually caged mice.

Physiological measurements. O₂ consumption, CO₂ release rates, and ambulatory activity levels were determined by using a 4-chamber Oxymax system (Columbus Instruments), with 1 mouse per chamber as previously described (Yu et al., 2000). Forced treadmill exercise test was performed by using the treadmill (Columbus Instruments) as previously described (Shalom-Barak et al., 2004). Muscle strength in mice was measured using an automated Grip Strength Meter (Columbus Instruments) as previously described (Acakpo-Satchivi et al., 1997).

MRI measurements. MRI was performed on a Bruker Avance 500 wide bore spectrometer (11.7 T; 500 MHz for proton) fitted with a gradient amplifier for imaging (Viereck et al., 2005). Data were processed with Paravision software provided by the vendor.

Metabolic measurements. Blood glucose was assayed with an Accu-check glucose monitor (Roche Diagnostics Corp.). Serum insulin was determined by enzyme-linked immunosorbent assay, using mouse insulin as a standard (Crystal Chem. Inc.). Glucose tolerance tests (GTT) was performed on 6 hours fasted mice. Mice were injected intraperitoneally with D-glucose (1 g/kg of body weight), and blood glucose levels were determined immediately before and at 30, 60, 90, and 120 min after injection. Glucose uptake in vivo skeletal muscle was determined as previously described (Koh et al., 2006). The rate of fatty acid β-oxidation in liver was examined as described previously (Nemoto et al., 2000).

Hindlimb Ischemia model. Mice were anaesthetized with a mixture of ketamine (80 mg/kg) and xyaline (10 mg.kg). The left femoral artery was ligated at the point of entry through the inguinal ligament, at the origin of the popliteal artery, and at midway through the saphenous artery. Small branches were cauterized, and the portion of the artery between the ligatures was removed. Blood flow was measured using a deep penetrating laser Dopper probe (Perimed) placed directly on the gastrocnemius muscle. Flow measurements were made just before, immediately after and at 2, and 4 weeks after femoral arteriectomy. At 4 weeks after femoral resection, gastrocnemius and soleus muscle form the ischemic and control limbs were fixed with methanol and embedded in paraffin. Five micron sections were stained with TRITC-labeled lectin (Bandeiraea Simplicifolia; Sigma-Aldrich).

Histology. Skeletal muscle and liver tissues were embedded in OCT compound (Sakura Finetech USA Inc) and snap-frozen in liquid nitrogen. White adipose tissues were fixed in 10% formalin, dehydrated, embedded in paraffin. Tissue sections were stained with H&E for overall morphology, masson-trichrome (MT) for fibrosis and Oil red-O for lipid deposition by standard methods.

Western blotting Western blot analysis was performed as described previously (Shiojima et al., 2002). The antibodies used were: phospho-Akt (ser-473) from Cell Signaling Technology; Akt1 and VP16 from Santa Cruz Biotechnology Inc.; HA (12CA5) from Roche Diagnostics Corp.; and tubulin from Calbiochem.

Quantitative real-time PCR. Total RNA from whole gastrocnemius muscle and liver was prepared by Qiagen using protocols provided by the manufacturer. cDNA was produced using ThermoScript RT-PCR Systems (Invitrogen). Real-time PCR was performed as described previously (Izumiya et al., 2006). Transcript levels were determined as the relative number of transcripts to those of 18S rRNA, and normalized to the mean value of control samples. Primer sequences are available upon request.

Statistical Analysis. All data are presented as mean±SEM. Statistical comparison of data from two experimental groups were made by using Student's t test. Comparison of data from multiple groups was made by ANOVA with Fisher's PLSD test. A level of P<0.05 was accepted as statistically significant.

### Example 1

### Skeletal Muscle-Specific Akt1 Transgenic Mice

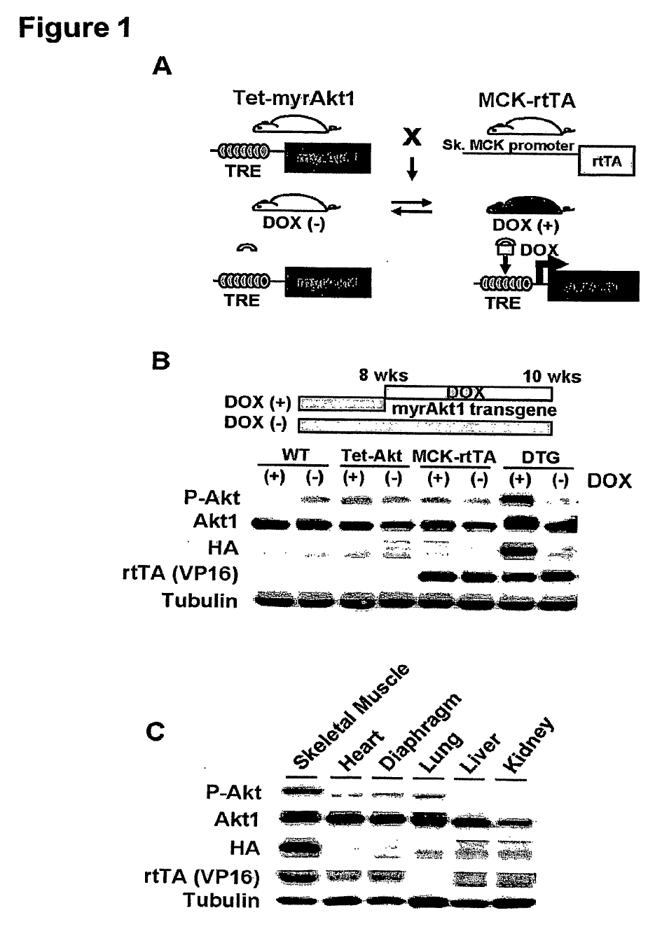

GENERATION OF SKELETAL MUSCLE-SPECIFIC INDUCIBLE AKT1 TG MICE: Two lines of TG mice (Tet-myrAkt1 and MCK-rtTA) were used to generate skeletal muscle-specific conditional Akt1 TG mice (FIG. 1A). Tet-myrAkt1 TG line harbours an active form of Akt1 (myrAkt1) transgene under the control of tetracycline responsive element (TRE) (Shiojima et al., 2005), and MCK-rtTA TG line expresses reverse tetracycline transactivator (rtTA: a fusion protein of TRE and VP16 transactivation domain) in the skeletal muscle driven by mutated MCK promoter (Grill et al., 2003). Treatment of double transgenic (DTG) mice harboring both of two transgenes with doxycycline (DOX) results in myrAkt1 transgene expression because DOX associates with rtTA enable to binding to TRE. On the other hand, withdrawal of DOX inhibited rtTA to bind to TRE and repression of myrAkt1 expression in the skeletal muscle. Mating of Tet-myrAkt1 mice and MCK-rtTA mice resulted in the generation of mice with four different genotypes (wild type (WT), Tet-myrAkt1 TG mice, MCK-rtTA TG mice and DTG mice) in expected frequencies. To examine the regulated expression of Akt1 transgene, these mice were divided into DOX (−) and DOX (+) groups: DOX (+) group was treated with normal water until the age of 8 weeks old followed by DOX treatment for 2 weeks, and DOX (−) group was treated with normal water until the age of 10 weeks old (FIG. 1B). Western blot analysis of gastrocnemius muscle lysates harvested at 10 weeks of age revealed that transgene expression detected by anti-HA blot was observed only in the DTG mice with DOX, indicating that the expression of Akt1 transgene in the skeletal muscle is tightly regulated in a DOX-dependent manner. The induced expression of Akt1 transgene was associated with marked increase in phosphorylation levels of Akt at Ser473, moderate increase in total Akt protein levels (FIG. 1B). As shown in FIG. 1C, Akt1 transgene expression was detected only in skeletal muscle, indicating that the expression of transgene was regulated skeletal muscle-specific manner. The inventors have also established the second Tet-myrAkt1 transgenic mouse line that exhibits relatively lower expression levels of Akt1 transgene (data not shown). Because the first Tet-myrAkt1 line exhibited more robust growth regulatory effects and permitted a better assessment of skeletal muscle growth and function, further experiments were performed using this line of Tet-myrAkt1 mice.

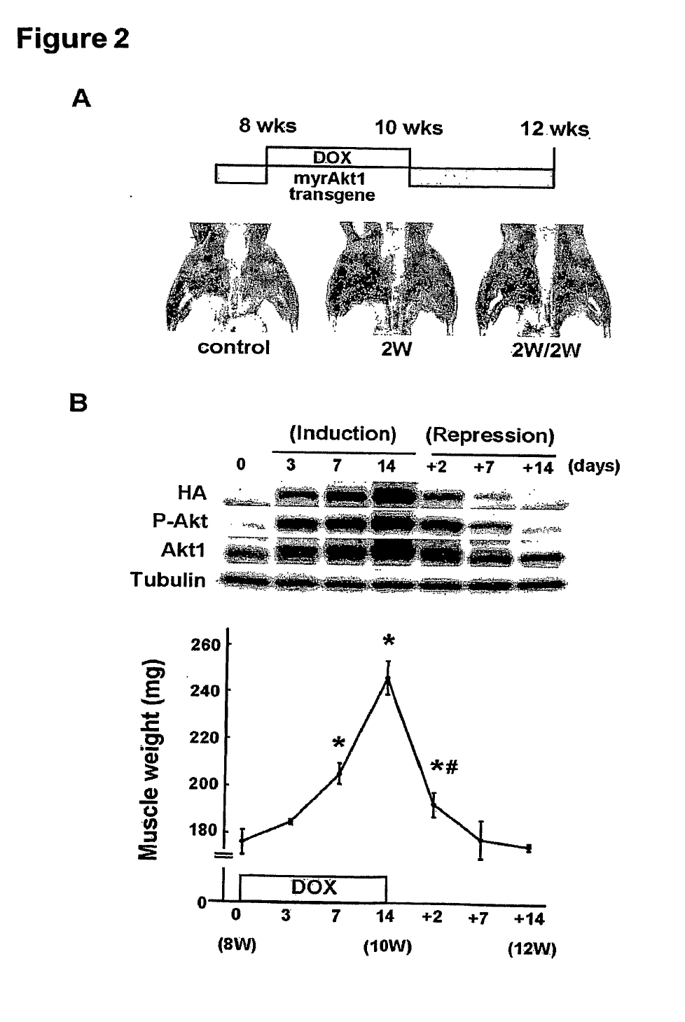

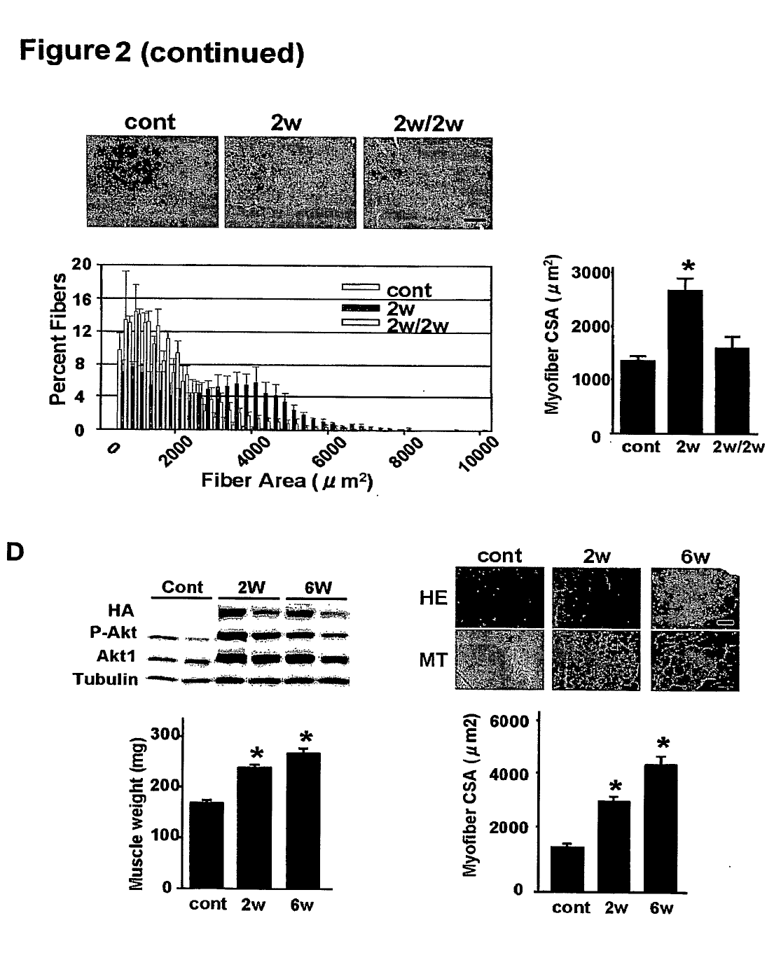

ACTIVATION OF AKT1N SKELETAL MUSCLE CAUSES FUNCTIONAL TYPE II MUSCLE FIBER HYPERTROPHY: To examine the consequence of Akt1 transgene expression in skeletal muscle, mice were treated with DOX at 8 weeks of age and transgene was induced for 2 weeks, and repressed it by removing DOX for 2 weeks. As shown in FIG. 2A, activation of Akt1 signaling for 2 weeks induced robust muscle growth. Akt 1-induced muscle growth was completely reversed 2 weeks after withdrawal of DOX. The time course of transgene expression and gastrocnemius muscle weight was examined (FIG. 2B). Akt1 transgene expression was first detected at day 3, which indicated that the initial transgene expression occurred immediately after DOX treatment. Transgene expression reached its maximal level on day 14. Marked repression of transgene expression was observed as early as 2 days after withdrawal of DOX, and transgene expression was completely suppressed by day 14 after withdrawal of DOX. Gastrocnemius muscle weight was significantly increased at day 7, and it was further increased at day 14. When DOX was withdrawal, dramatically repression of hypertrophy was observed at 2 days after withdrawal of DOX. And gastrocnemius muscle weight was almost completely reversed to basal level at 7 days after withdrawal of DOX.

Histological analysis revealed that individual myofiber size was apparently increased, and it was reversed after transgene repression (FIG. 2C). An analysis of the individual fiber sizes demonstrates a significant shift to the right in gastrocnemius muscle. Average cross sectional area was about 2-fold increased 2 weeks after Akt1 activation (2657±195 vs. 1338±80 μm2, p<0.01). Maintaining the transgene expression for 6 weeks induced further skeletal muscle hypertrophy (FIG. 2D). Levels of Akt1 transgene expression was similar extent between 2 weeks and 6 weeks after induction. Gastrocnemius muscle weight was significantly higher than that of control at both time points. As revealed by histology, prolonged Akt1 activation in skeletal muscle induced more pronounced muscle hypertrophy without any pathological change such as interstitial fibrosis or inflammation.

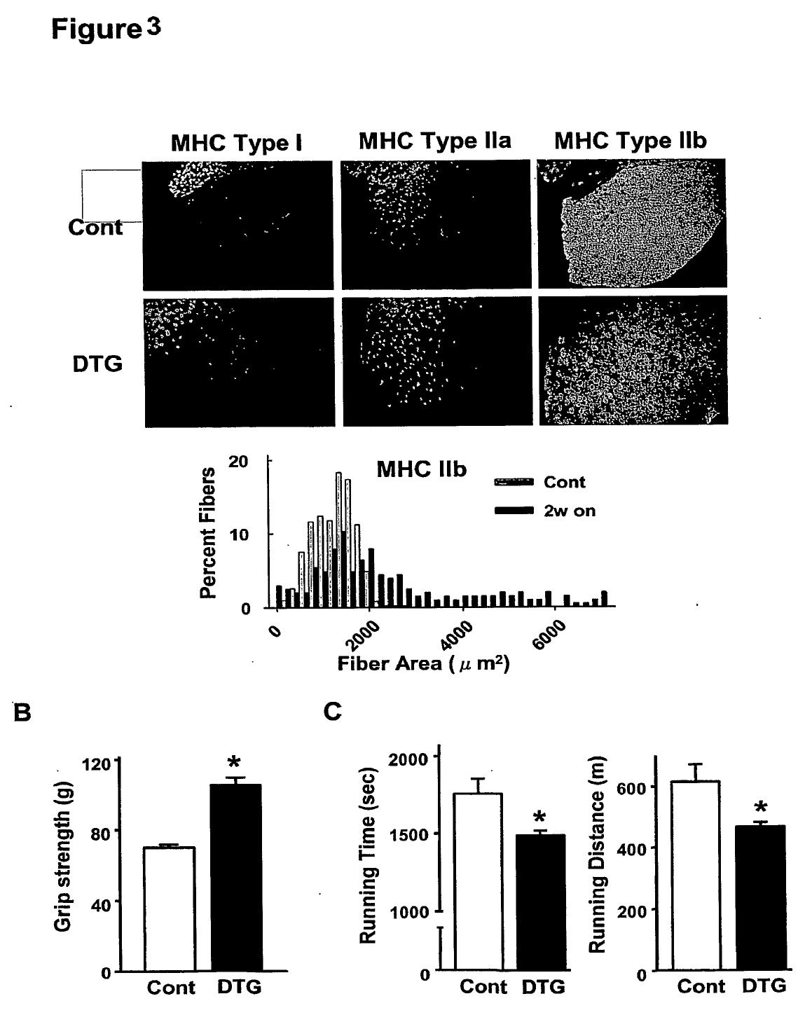

PHYSICAL PERFORMANCE OF AKT-MEDIATED HYPERTROPHIED ADULT SKELETAL MUSCLE: To examine which muscle fiber preferentially express Akt1 transgene, gastrocnemius muscle sections were stained with anti-HA and MHC isoform antibody. As shown in FIG. 3A, Akt1 transgene detected by anti-HA antibody was preferentially expressed in type IIb fibers. Type IIb fibers are classified as fast/glycolytic muscle which is responsible for force generation. Consistent with Akt1 transgene expression profile, the peak grip force for the DTG was about 50% greater than that of control 2 weeks after Akt1 induction (104.7±3.7 vs. 69.8±0.8 g, p<0.05). On the other hand, forced treadmill exercise test revealed that DTG has less running capacity than control (FIG. 3B). These results reveal that Akt1 activation in type II muscle fibers enable to generate of mice strain with “resistance training” phenotype.

AKT1-MEDIATED TYPE H MUSCLE FIBER GROWTH REGRESS DIET-INDUCED OBESITY AND OBESITY-RELATED METABOLIC DISORDERS: To investigate the relationships between type II muscle growth and obesity, mice were fed high fat/sucrose (HF/HS) diet to induce obesity. Under conditions of repressed Akt1 activation, no significant difference was observed in body weight gain in control and DTG mice fed HF/HS diet (FIG. 4A). However, once Akt1 was activated in type II skeletal muscle of obese mice, body weight was dramatically decreased compared with control mice. MRI analysis revealed that accumulation of excessive amount of fat was significantly decreased in DTG mice (FIG. 4B). Histological analysis revealed that myofiber hypertrophy was obvious in DTG mice (FIG. 4C). Adipocyte cell size was enlarged by HF/HS diet in control mice, however, it was apparently smaller in DTG mice. Gastrocnemius muscle weight was significantly increased in DTG mice compared with control mice (FIG. 4D). Inguinal fat pad weight was increase by HF/HS diet in control mice, however, it was dramatically reduced in DTG mice (FIG. 4D).

The inventors next examined the effect of Akt1-mediated type II muscle growth on whole body glucose metabolism. There is no difference in blood glucose levels in each group in fasting period, however, it was significantly higher in BF/HS diet fed control mice in fed period (FIG. 5A). Fasting serum insulin level was significantly increased only in control mice fed HF/HS diet, indicating that these mice developed insulin resistance and Akt1-mediated type II muscle growth improved insulin resistance (FIG. 5B). To investigate this point further, we performed glucose tolerance test (GTT). As shown in FIG. 5C, DTG mice fed a HF/HS diet maintained glucose levels similar to those in mice on normal diet after GTT, whereas control mice fed a HF/HS diet showed higher glucose levels after injection. These results indicate that HF/HS diet-induced severe glucose intolerance was clearly improved after Akt1 activation in type II skeletal muscle. To examine whether the improved glucose tolerance is owing to increased glucose disposal in skeletal muscle, we measured skeletal muscle glucose uptake in vivo and found that muscle glucose uptake was 1.6-fold and 2.0-fold higher in DTG fed normal diet and HF/HS diet, respectively (FIG. 5D).

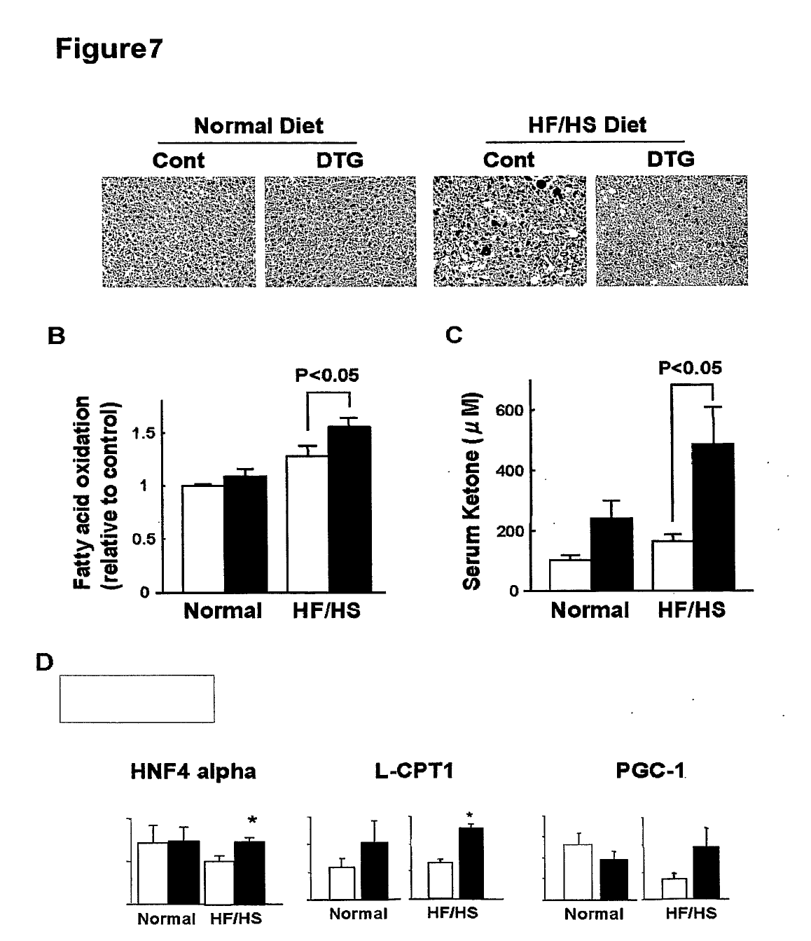

To investigate the mechanism by which excessive fat accumulation was reversed by type II muscle growth, the inventors examined the energy balance: food intake and energy expenditure. Both control and DTG mice show similar food intake throughout the experimental period (FIG. 6A). Although ambulatory activity levels of HF/HS diet fed DTG mice was ˜40% lower than that of control mice (FIG. 6B), energy expenditure estimated by whole-body O2 consumption (VO2) was significantly higher than that of control mice (FIG. 6C). Respiratory exchange ratio (RER), which reflects the ratio of carbohydrate to fatty acid oxidation, was significantly decreased in HF/HS diet fed DTG mice indicating that these mice using a relative greater ratio of fatty acid as a fuel source during the fasting period. However, quantitative real-time PCR analysis revealed that most of genes associated with fatty acid oxidation and mitochondrial biogenesis were not upregulated in skeletal muscle (Table 1). Because liver is a metabolically active organ as well as skeletal muscle, we checked liver morphology and lipid oxidative function. As revealed by histology, HF/HS diet-induced lipid deposition in liver was dramatically resolved in DTG mice (FIG. 7A). To investigate why lipid deposition was decreased in DTG mice, we measured fatty acid oxidation in vivo liver and found that greater fatty acid was oxidized in liver in HF/HS diet fed DTG mice (FIG. 7B). Serum ketone bodies, which synthesized in the liver and can be used as an indirect marker of hepatic fatty acid oxidation, was significantly increased in HF/HS diet fed DTG mice (FIG. 7C). Finally, quantitative real-time PCR analysis showed significant increase in expression of HNF4α, L-CPT1 and PGC1-α in liver in HF/HS diet fed DTG mice, suggesting that Akt1-mediated type II muscle growth activates molecules involved in stimulating fatty acid oxidation in liver.

TABLE 1
Gene Expression associated with energy expenditure in
skeletal muscle in HF/HS fed control or DTG mice.

| Gene | Cont | DTG |
|---|---|---|
| Akt1 | 0.8 ± 0.07 | 9.3 ± 3.92* |
| Cytochrome c | 1.4 ± 0.06 | 1.3 ± 0.04 |
| COX II | 1.4 ± 0.13 | 1.6 ± 0.22 |
| COX IV | 1.8 ± 0.24 | 1.8 ± 0.18 |
| CPT1 | 0.8 ± 0.10 | 1.1 ± 0.09 |
| FATP | 0.9 ± 0.07 | 0.6 ± 0.06 |
| MCAD | 1.0 ± 0.14 | 0.9 ± 0.11 |
| UCP2 | 1.1 ± 0.16 | 4.5 ± 0.82* |
| UCP3 | 0.9 ± 0.14 | 0.9 ± 0.18 |
| PGC1-α | 1.0 ± 0.08 | 0.5 ± 0.05* |
| PPARα | 1.3 ± 0.30 | 0.5 ± 0.03* |
| PPARδ | 1.0 ± 0.04 | 0.8 ± 0.13 |

Values are fold change vs. normal diet control.
*= p < 0.005 vs. HF/HS Diet control.

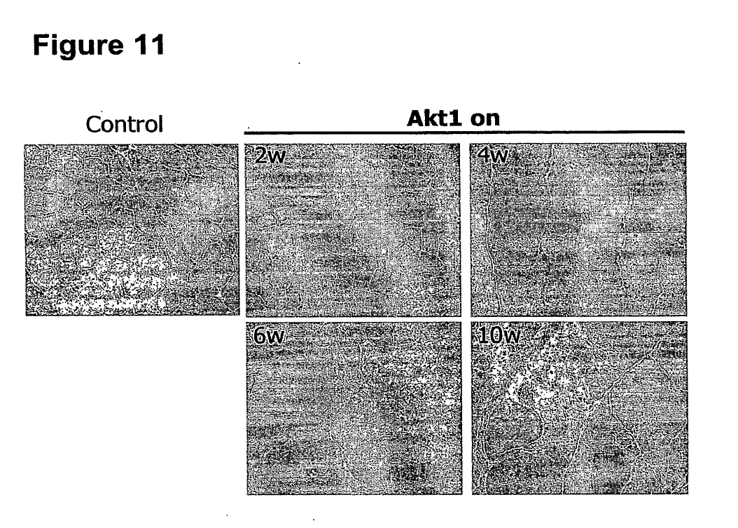

In summary, myogenic Akt transgene activation in obese mice confers the following phenotype: 1) increased muscle mass and strength, 2) diminished fat mass, 3) diminished body weight, 4) improved insulin sensitivity, 5) diminished steatosis (fatty liver), 6) increased angiogenesis in skeletal muscle and 7) increased muscle growth via the incorporation of satellite cells (FIG. 11). These effects occur despite constant levels of food intake and physical activity.

In the present study, the inventors have discovered that type II skeletal muscle growth regress obesity and obesity-related metabolic disorders in obese mice. Akt1 activation in type II skeletal muscle dramatically induced muscle hypertrophy, which was accompanied by an apparent reduction in body weight, especially in fat mass, as well as an improvement of glucose intolerance induced by HF/HS diet. These effects were achieved without dietary and activity modifications. Furthermore, type II skeletal muscle growth led to increased fatty acid oxidation and decreased lipid deposit in liver. Because type II muscle fibers are dramatically decreased upon aging (Larsson, 1983), and cross-sectional studies revealed that muscle strength is inversely correlated with the prevalence of metabolic syndrome (Jurca et al., 2005; Jurca et al., 2004), the inventors have discovered that resistance training aimed at increasing type II muscle fibers is beneficial intervention for the patients, particularly in elderly, with obesity and obesity-related metabolism.

In the present study, the inventors have discovered that type II muscle fiber growth led to increase whole-body energy expenditure independent of physical activity levels in obese mice (FIGS. 6B, C). This discovery indicates that building and maintaining type II muscle per se energy expensive. Reduced energy supplies to adipose tissue as a result of increase energy demand from large skeletal muscles might contribute to regression of obesity. Furthermore, the inventors have discovered that Akt1 activation in type II muscle fibers significantly increased glucose uptake into skeletal muscle, and impaired glucose tolerance induced by HF/HS diet was dramatically improved by Akt1 activation in type II muscle (FIG. 5). Therefore, the inventors have discovered that Akt1-mediated type II fiber growth leads to improvement of HF/HS diet-induced glucose intolerance. Another remarkable discovery disclosed herein is that HF/HS diet-induced liver seatosis was dramatically resolved and fatty acid oxidation was significantly increased in liver in DTG mice (FIG. 7), indicating that activation of Akt1 signaling in skeletal muscle produces some secreted factor and directly affect liver in a paracrine manner. In a previous study, the inventors have previously reported that Akt1 activation in skeletal or cardiac muscle induced muscle hypertrophy and coordinated blood vessel recruitment by secreting pro-angiogenic factors released from myocyte (Shiojima et al., 2005; Takahashi et al., 2002). The discovery herein indicates that Akt1-mediated type II muscle growth induce coordinated regulation of glucose/lipid metabolism with other organs.

In conclusion, the inventors have discovered for the first time that type II skeletal muscle growth improves obesity-related metabolism by modulating lipid oxidation in liver. This discovery provides a novel concept that type II skeletal muscle fibers regulate glucose/lipid metabolism by communicating remote organs.

### Example 2

### Detailed Characterization of Activation of Akt1 in Skeletal Muscle

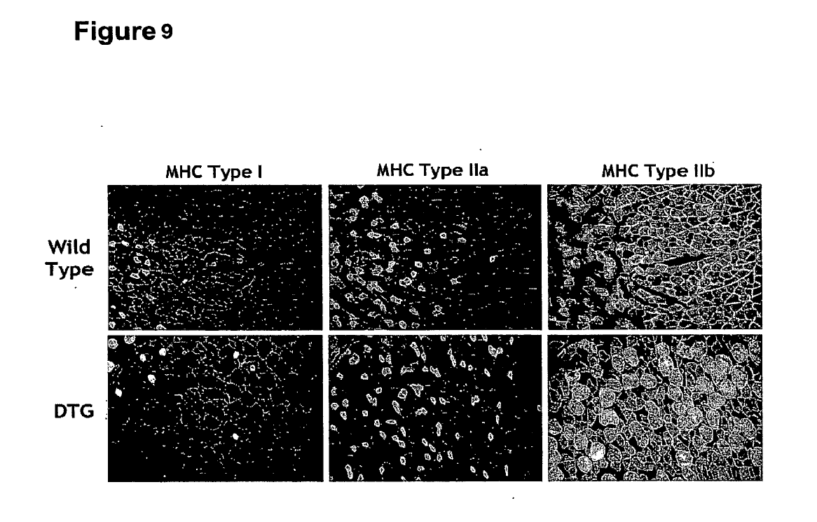

Transgenic mice with inducible expression of Akt in muscle were further characterized, as shown in FIGS. 9-12. When expression of Akt was induced by administration of DOX(DTG) to the drinking water hypertrophy of Type IIb muscle fibers, typically glyolytic/fast twitch fibers is seen compared to wild type mice, and less Type I and Type IIa fibers occur in DOX treated Akt mice compared to control.

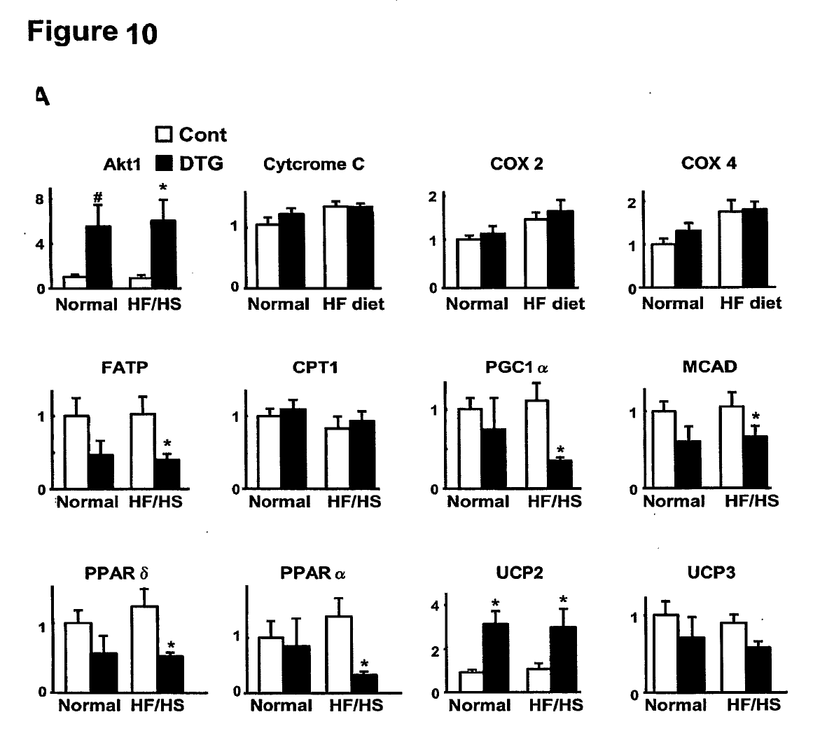

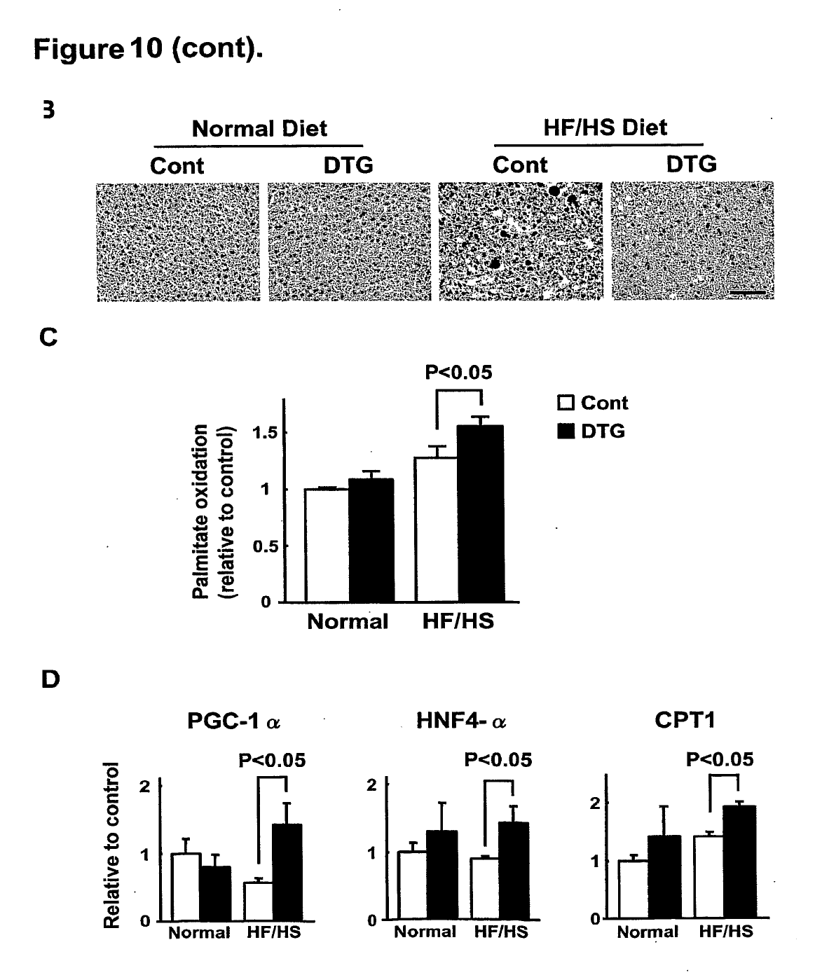

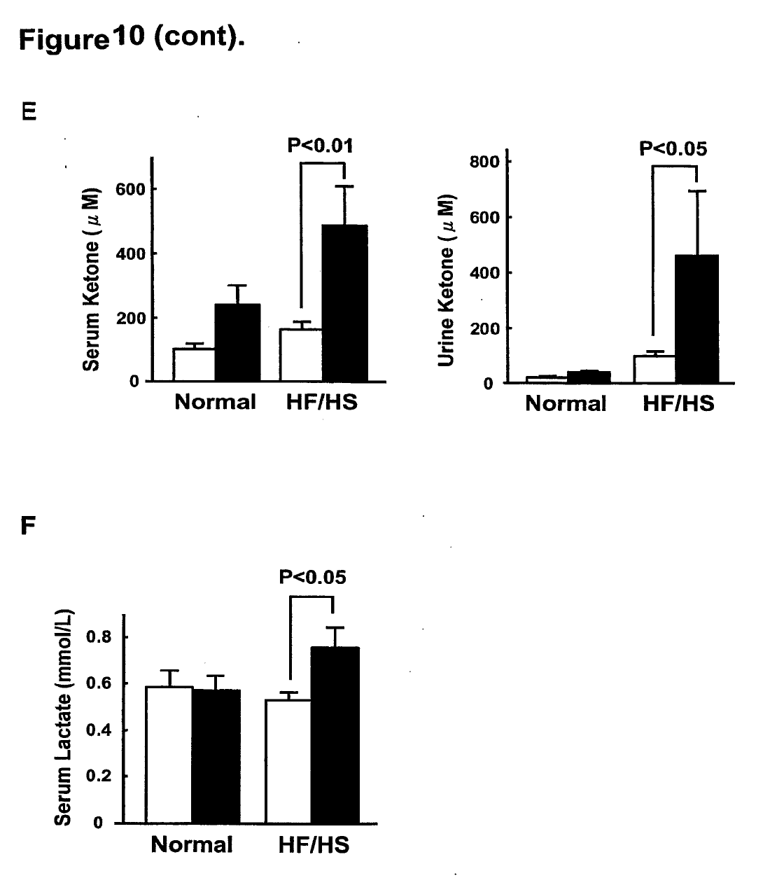

Transgenic Akt mice fed DOX fed high fat and high sugar (HF/HS) also have increased lipid peroxidation in the liver but not muscle compared to control mice fed HF/HS diet, as detected by quantitative gene expression analysis if a number of mRNAs associated with fatty acid oxidation and mitochondrial biogenesis (FIG. 11A) and increase in total fatty acid β-oxidation of palmitic acid (FIG. 10C), and also morphological analysis of liver using oil red-O stain (FIGS. 7A and 10B). In Akt transgenic mice fed HF/HS diet, PGC-1, HNF4-α and CPT-1, genes associated with fatty acid oxidation are increased (FIG. 10D) as well as increases in increases in serum and urine ketone bodies (FIG. 10E) and serum lacatate levels (FIG. 10F) compared to HF/HS fed control mice, indicating Akt activation in the muscle increases fatty acid metabolism in the liver as well.

Analysis of the effect of induction of Akt expression in the muscle on other tissues can be evaluated using differential gene expression analysis, for example using tissues from liver, adipose cells, and other organs, for example kidney, spleen, pancreas, nervous system tissue etc (see FIG. 12),

### Example 3

INDUCIBLE EXPRESSION OF AKT1 IN VITRO: Transduction of cells in vitro or tissues in vivo with Akt1, Akt2 or Akt3 (constitutively-active or dominant-negative forms) should lead to similar changes in transcript levels because we have shown previously that Akt2 (Fujio Y. et al. 2001 Cell Death Duff 8:1207-1212), and Akt3 (Y. Taniyama 2005 J Mol Cell Cardiol 38:375-385) share function properties with Akt1.

To examine if activation of Akt in skeletal cells in vitro leads to similar changes in gene expression, a myogenic cell line, C2C12 cells, was transduced with an adenovirus expressing Akt1 (Adv-myrAkt). Comparison of gene expression of Adv-myrAkt cells transfected cells 1 day after with skeletal muscle cells of induced expression of Akt1 in skeletal muscle in mice have a highly similar gene expression profile, indicating skeletal muscle cells expressing Akt1 cultured in vitro are effective tools for studying muscle secreted proteins (MSP) or myokines

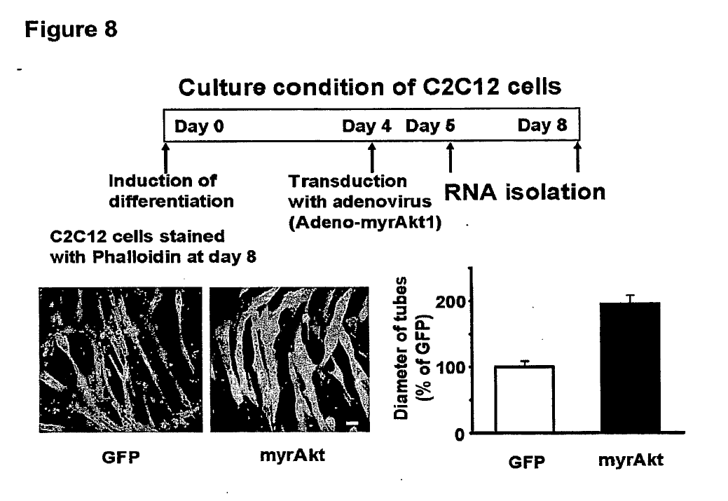

As shown in FIG. 8, the a protocol was devised to identify muscle secreted proteins (MSPs) in an in vitro assay, by expressing myrAkt in a skeletal muscle cell line, for example C2C12. Other skeletal muscle cell lines can be utilized, for example human skeletal muscle cell lines.

### Example 4

IDENTIFICATION OF FACTORS ASSOCIATED WITH MUSCLE GROWTH, ANGLIOGENEIS, OBESITY, INSULIN SENSITIVITY AND CARDIOVASCULAR FUNCTION: A protocol was devised to identify novel muscle secreted proteins (MSPs) that confer the phenotypes, for example, but not limited to increased glucose sensitivity and insulin sensitivity, decreased fat mass and decrease fat cell size, decreased liver deposition, increased capillary density and increased satellite cell recruitment and incorporation of satellite cells into the fibers. First, we performed microarray analysis on muscle of control and DTG mice. Total RNA from gastrocnemius muscle of inducible Akt transgenic mouse (3 groups; no transgene induction (control), 2 weeks induction (2w on), and 2 weeks induction/2 days repression (2w on/2d off)) was analyzed by Affymetrix GeneChip® Mouse Expression Set 430 microarrays. Among the transcripts upregulated by Akt induction, unknown genes were selected which have full-length open reading frame cDNAs available in the NCBI website. Predicted amino acid sequences were then examined for putative signal sequences using Signal IP software. Unknown transcripts with predicted signal sequences were then analyzed with SOSULI signal beta version software to predict whether they encode for secreted proteins versus an integral membrane proteins. This subset of cDNAs was then validated by real-time PCR in the gastrocnemius muscle of DTG mice in the presence or absence of DOX

Based upon the above examination, 8 selected cDNAs were further analyzed to test whether the gene products are secreted by mammalian cells. Full-length cDNAs were obtained by PCR, and subcloned into pcDNA3.1/V5-His that express the unknown protein as a fusion to the V5 epitope in the N-terminus and His tag in C-terminus for detection. These expression vectors were then transfected into HEK293 cells. After 2 days the cell pellets and media fractions were collected and analyze by western blot using anti-V5 antibody. Where recombinant protein can be detected both in the cell pellet and in the medium, indicating that this cDNA encodes a secreted protein. In all, 6 of the 8 cDNAs encoded secreted proteins as assessed by this HEK293 cell transfection assay, whereas 2 cDNAs did not (i.e. protein could be detected in the cell pellet but not media).

In summary, based upon microarray analysis and transfection assays as described above, the inventors have identified 6 novel cDNAs encoding secreted proteins that are upregulated during Akt-mediated muscle growth (Table 2). These factors are referred to as muscle-secreted proteins 1-6 (MSP 1-6). The Riken identification numbers and the GenBank Accession Numbers for MSP 1-5 are described in Table 2. MSP6 is FGF21, a metabolic factor that may have utility for diabetes and obesity (J. Clin. Invest. 2005; 115:1627-1635).

TABLE 2
Muscle secreted proteins

| | Akt transgene | | | |
|---|---|---|---|---|
| | 2w | 2w on/ | | |
| Gene | on | 2d off | Secretion | Homology |
| MSP 1 | 6.8 | 3.8 | yes | Thrombospondin, type I like |
| MSP 2 | 4.7 | 2.9 | yes | None |
| MSP 3 | 15.1 | 7.5 | yes | Coiled coil domain |
| MSP 4 | 2.4 | 1.1 | yes | peptidase M20 domain |
| MSP 5 | 1.9 | 0.7 | yes | calcium-binding |
| | | | | EGF-like domain |
| MSP 6 | 68.0 | 5.8 | yes | similar to fibulin-1 C (N-term) |
| (FGF21) | | | | |

MSP1 corresponds to 2610028F08Rik (GenBank Accession No. BC052844) (SEQ ID NO:16), MSP2 corresponds to 2310043I08Rik (GenBank Accession No. AK009779) (SEQ ID NO: 17), MSP3 corresponds to 1110017I16Rik (GenBank Accession No. NM_{—}026754) (SEQ ID NO:1), MSP4 corresponds to 4732466D17Rik (GenBank Accession No. BC025830, AK028883) (SEQ ID NO: 18), and MSP5 corresponds to 1600015H20Rik (GenBank Accession No. AK005465) (SEQ ID NO: 12).

### Example 5

### MSP3 as Metabolic Regulator

PRODUCTION OF ADENOVIRAL VECTORS EXPRESSING MSPS AND EFFICICY IN ISCHEMIC HIND LIMB ANGIOGENESIS ASSAY: Adenoviral expression vectors to all six MSP cDNAs were produced by homologous recombination in HEK 293 cells as described previously (Mol. Cell. Biol. 2002; 22:680-691). In brief, MSP cDNAs were subcloned into an adenovirus shuttle vector designated Adeno-MSPs. Shuttle vector containing the MSP cDNAs were linearized and cotransformed into Escherichia coli with the adenoviral backbone plasmid pAdEasy-1. The resultant recombinant adenoviral DNA with MSP cDNAs were transfected into packaging cell line 293 cells to produce the recombinant adenoviral vectors. All viral constructs were amplified in 293 cells and purified by CsCl ultracentrifugation that is routine in the lab (Mol. Cell. Biol. 2002; 22:680-691; J. Biol. Chem. 2005; 280:20814-20823).

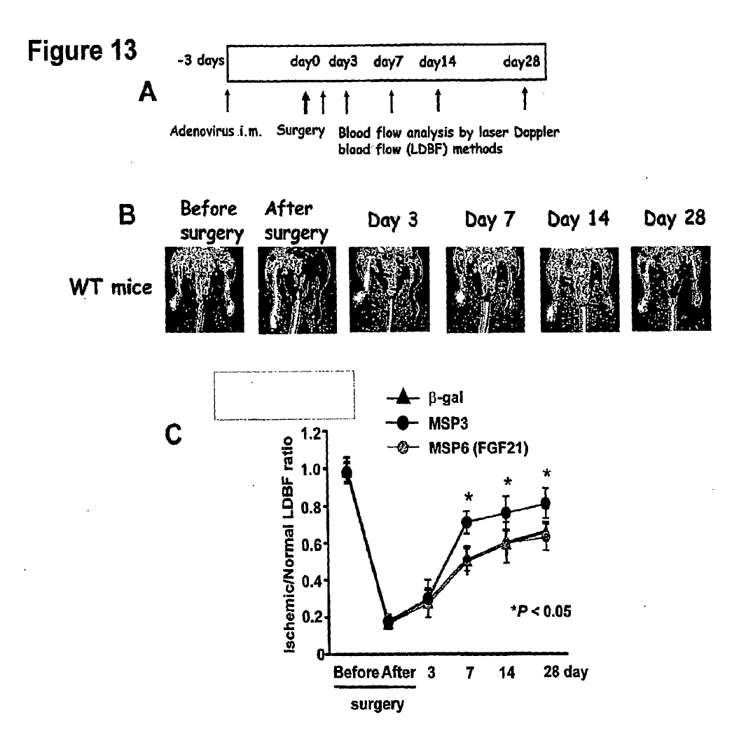

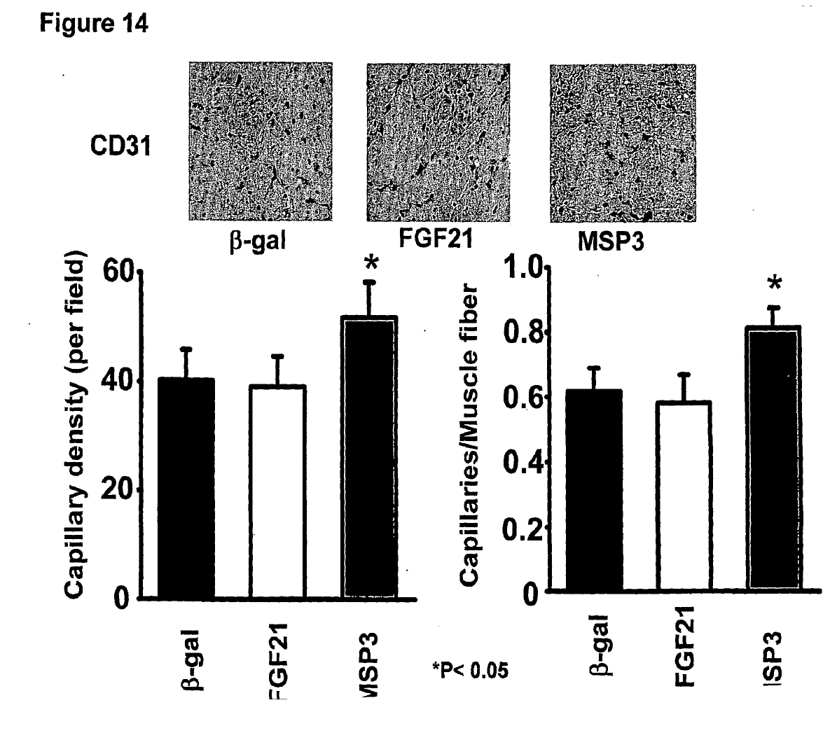

MSP3 corresponds to 1110017I16Rik (GenBank Accession No. NM_{—}026754) (SEQ ID NO: 1) and was discovered to be differentially regulated in response to expression of muscle related transgenes and muscle growth. To evaluate the potential angiogenic properties of two MSPs in vivo, MSP3 and MSP6 (FGF-21), mice at the ages of 10 weeks were subjected to unilateral hind limb surgery (J. Biol. Chem. 2004; 279:28670-28674; Circ. Res. 2005; 96(8):838-846; Circ. Res. 2006; 98(2):254-61). Adenovirus-mediated gene transfer was performed with adenoviral vectors expressing MSP3 and MSP6 by direct injection into five different sites of adductor muscle in the ischemic limb 3 days before surgery. Blood vessel growth was monitored by Laser Doppler analysis on legs and feet immediately before surgery and on postoperative days 0, 3, 7, 14, and 28 (FIG. 13). As shown in FIG. 13, adeno-MSP3-treated mice showed a significant increase in flow recovery at 7, 14, and 28 days after hind limb surgery as determined by laser Doppler blood flow analysis. On the other hands, adeno-MSP6 did not affect on flow recovery compared to control mice. These results suggest that MSP3, but not MSP6 (i.e. FGF21), functions as an angiogenesis-regulatory protein. This is also shown in FIG. 14, where Adv-MSP3 improves capillary density and microvessel formation, as identified by CD31 immunostaining, and quantitative analysis of capillary density compared to Adv-FGF21 or control Adv-β-gal treated mice.

MSP3 AS A METABOLIC REGULATOR OF GLUCOSE SENSITIVITY AND REGRESSES DIET-INDUCED OBESITY AND OBESITY-RELATED METABOLIC DISORDERS:

A protocol to assess diet-induced obesity model to test MSP metabolic function was established, in which mice fed a high fat, high sucrose diet are injected intramuscularily with Adenovirus (Adv) expressing MSP3 (at 1×10¹⁰ pfu) and body weight assessed at 7, 14, 21 and 28 days after Adv-injection, and blood glucose assessed at 14 and 28 days.

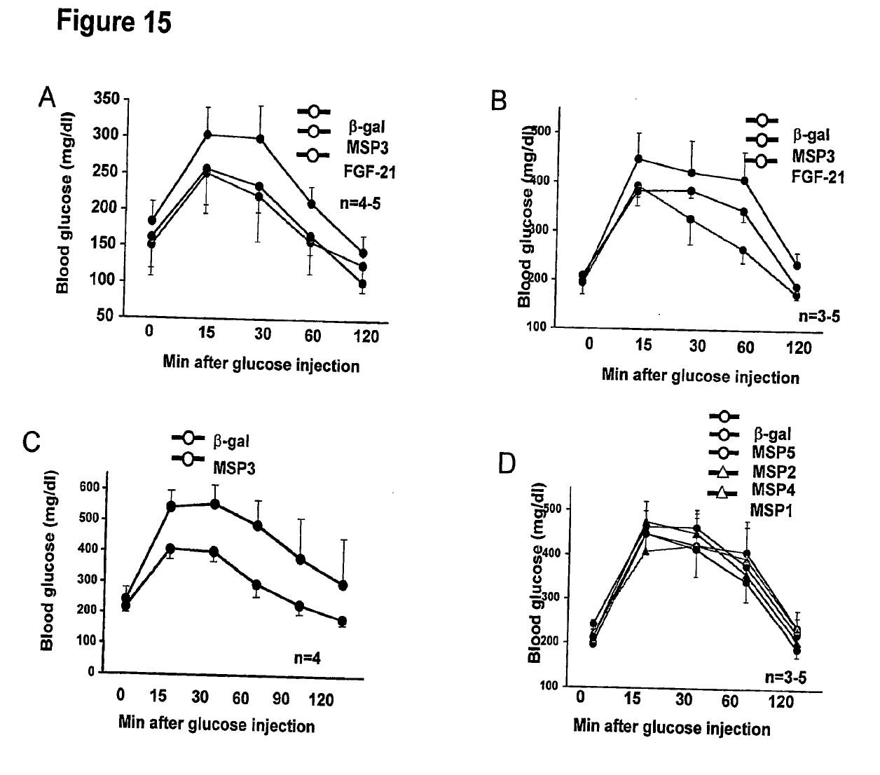

On Intramuscular injection of Adeno-MSP3, diet induced obesity mice had improved metabolic response and glucose sensitivity compared to Adv-β-gal injected mice (FIGS. 15A and B). Adenovirus-encoded MSP3 appears functionally equivalent to adenovirus-delivered FGF-21 (also known as MSP6), with blood glucose (mg/dl) returning to the same level with Adv-MSP3 and Adv-FGF21 by 120 minutes after glucose injection. Furthermore, this improved metabolic response and glucose sensitivity was not observed for other MSPS; (MSP5, MSP2, MSP4 and MSP1) (FIG. 15D).

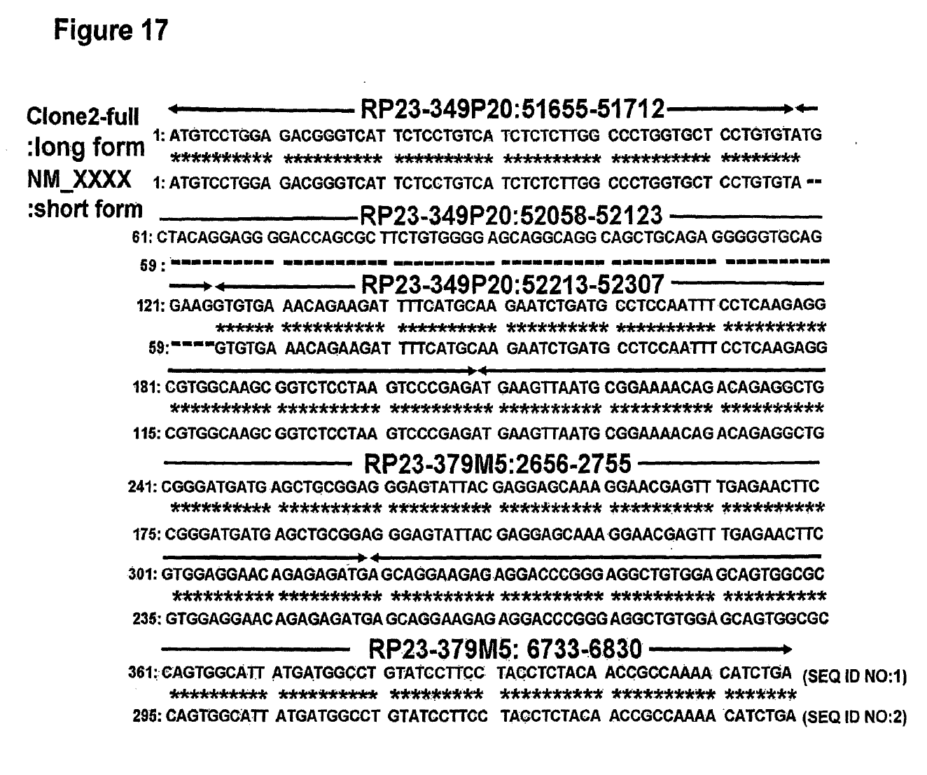

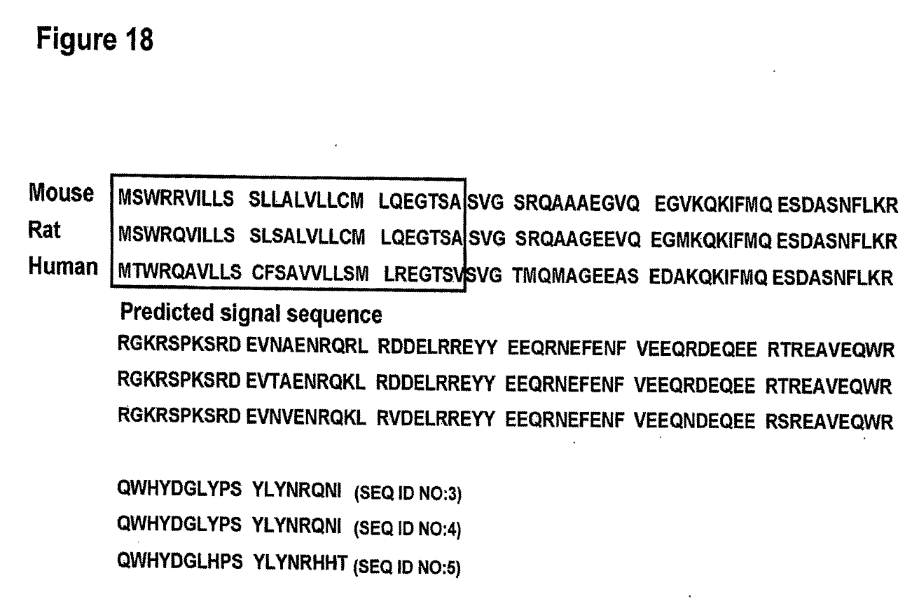

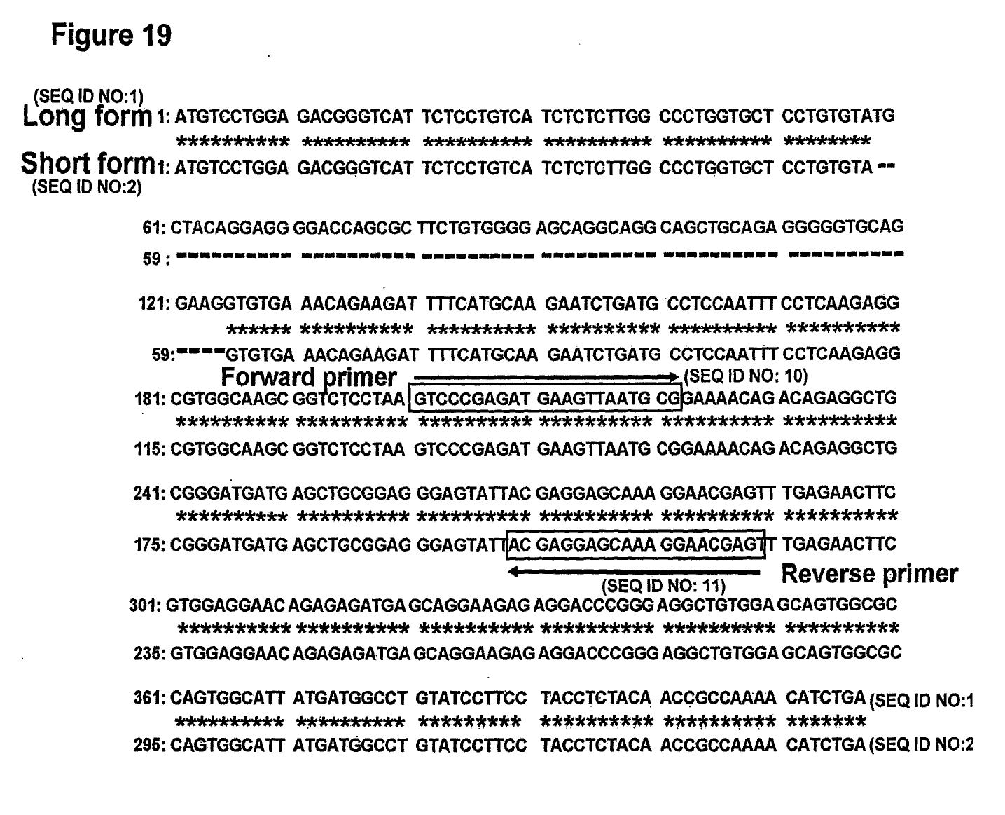

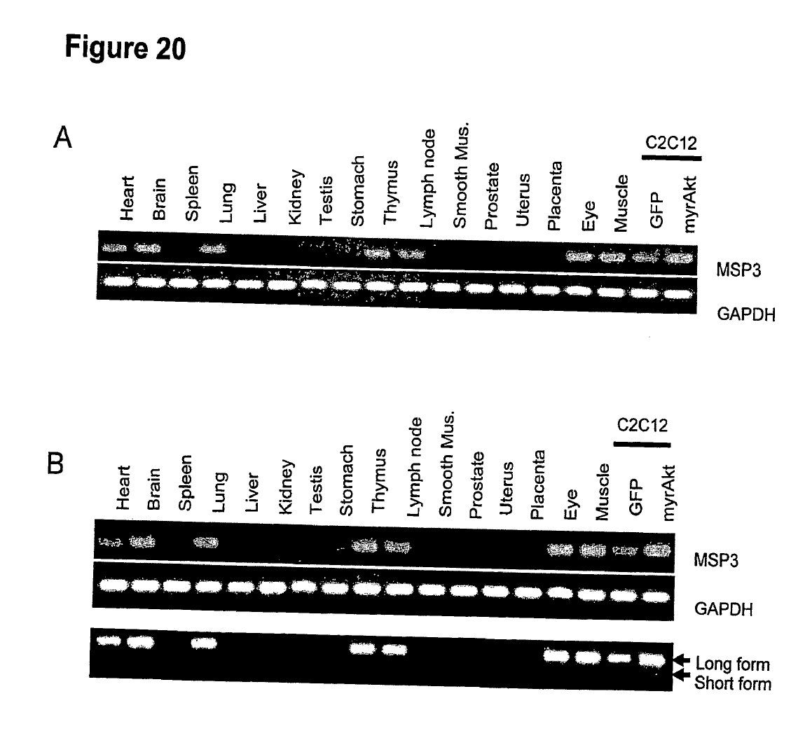

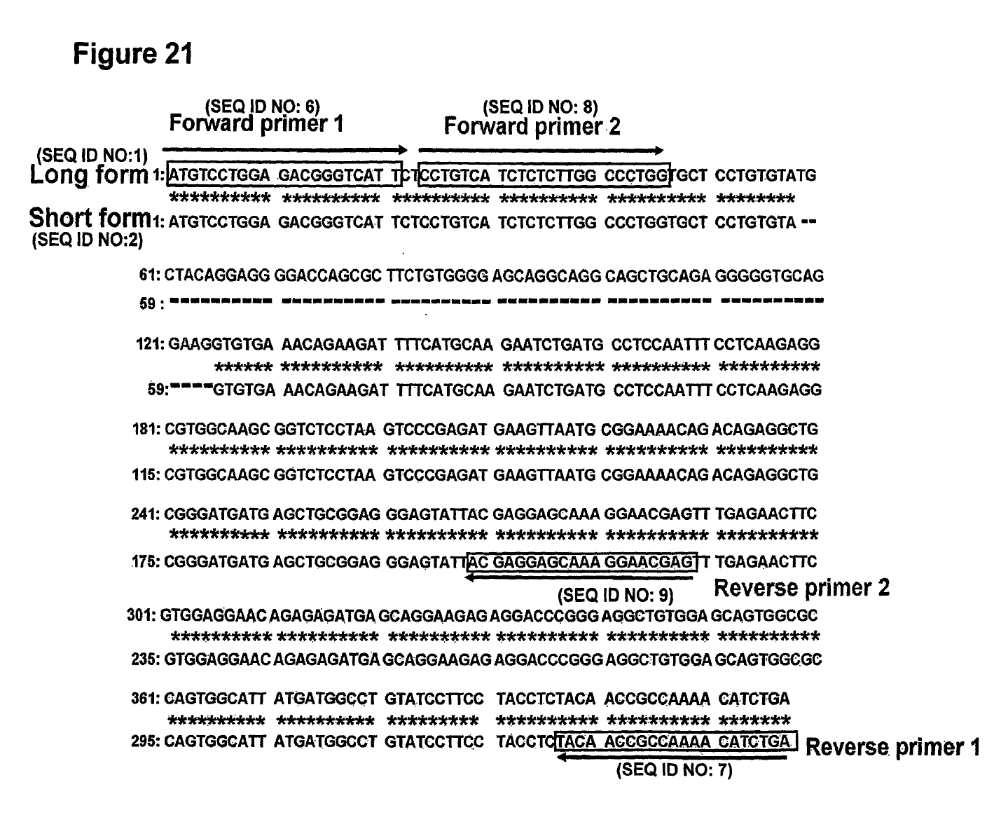

Quantities RT-PCR was performed to analyze the expression profile of MSP2, which is located on Chromosome 2 (FIG. 17) and exists as two alternatively spliced isoforms; a long isoform (SEQ ID NO: 1) and a short isoform (SEQ ID NO:2) (FIG. 16), and analysis of the amino acid sequences predicts MSP3 has a signal sequence and high homology between rodent and human isoforms; the sequence identity between mouse (SEQ ID NO: 3) and rat (SEQ ID NO:4) is 94% and the sequence identity between mouse and human (SEQ ID NO:5) is 79% (FIG. 18). Analysis of total MSP3 expression using primers designed to detect both the long and short isoforms (SEQ ID NOS: 10 and 11, FIG. 19), shows MSP3 has a restricted expression in heart, brain, lung, thymus, lymph node, eye and skeletal muscle (FIG. 20A). In addition, MSP3 was expressed in C2C12 cells, a myocyte cell line, and was expression was further induced by transfection of C2C12 cells with adenovirus expressing constitutively active Akt (MyrAkt) (FIG. 20A). Analysis of the expression of long and short isoforms of MSP3 was done using isoform-specific primers to each of the long and short isoforms (SEQ ID NOS: 6-9, FIG. 21), shows the long form of MSP3 is predominantly expressed (upper band) comparatively to the short form (lower band) (FIG. 20B).

In summary, the inventors have discovered that MSP3 has a dual function as a metabolic regulator that is not shared by FGF2, as MSP3 functions as an angiogenesis factor (FIGS. 13 and 14), and also regulates sensitivity to glucose in obese mouse model (FIG. 15), whereas FGF21 only functions to regulate sensitivity to glucose.

### Example 6

### MPS5 as a Muscle Hypertrophy Factor (Myogenic Factor)

MSP5 was discovered to be differentially regulated in response to expression of muscle related transgenes and muscle growth. MSP5 corresponds to 1600015H20Rik (GenBank Accession No. AK005465) (SEQ ID NO: 12)

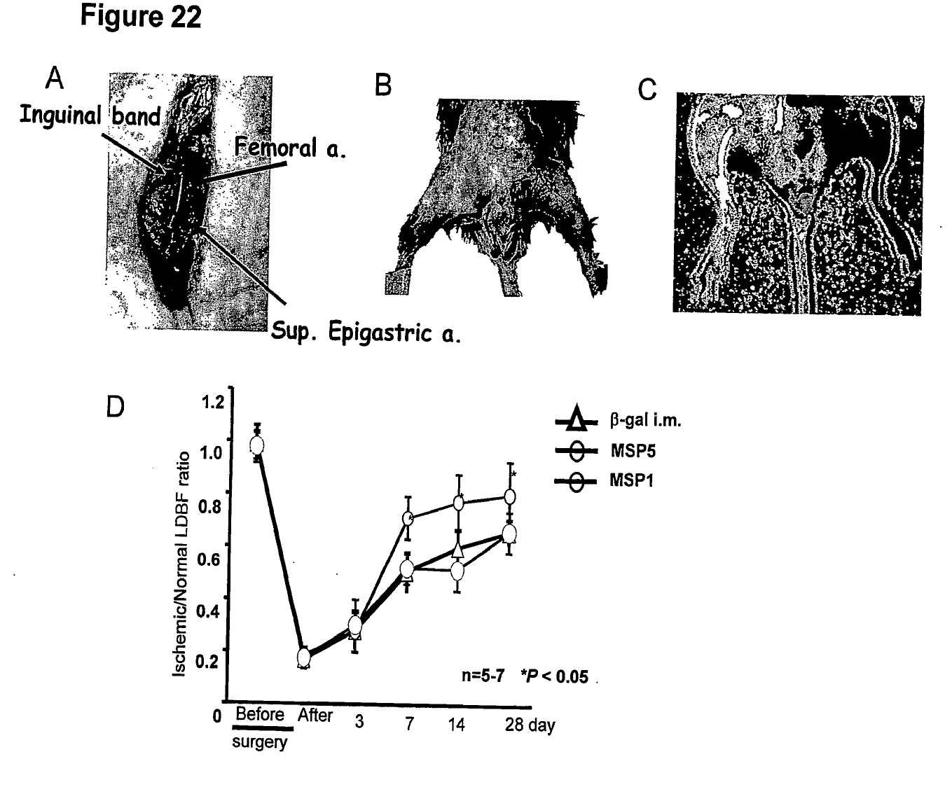

To evaluate the potential angiogenic properties of MSP5, mice at the ages of 10 weeks were subjected to unilateral hind limb surgery (J. Biol. Chem. 2004; 279:28670-28674; Circ. Res. 2005; 96(8):838-846; Circ. Res. 2006; 98(2):254-61). Adenovirus-mediated gene transfer was performed with adenoviral vectors expressing MSP5 and MSP1 by direct injection into five different sites of adductor muscle in the ischemic limb 3 days before surgery. Blood vessel growth was monitored by Laser Doppler analysis on legs and feet immediately before surgery and on postoperative days 0, 3, 7, 14, and 28 as in previous experiments, and Adv-MSP5 transfected mice had improved ischemic/Normal LDBF ratio as compared to β-gal control or Adv-MSP1 transfected mice (FIG. 22), indicating MSP5, but not MSP1 functions to increase angiogenesis.

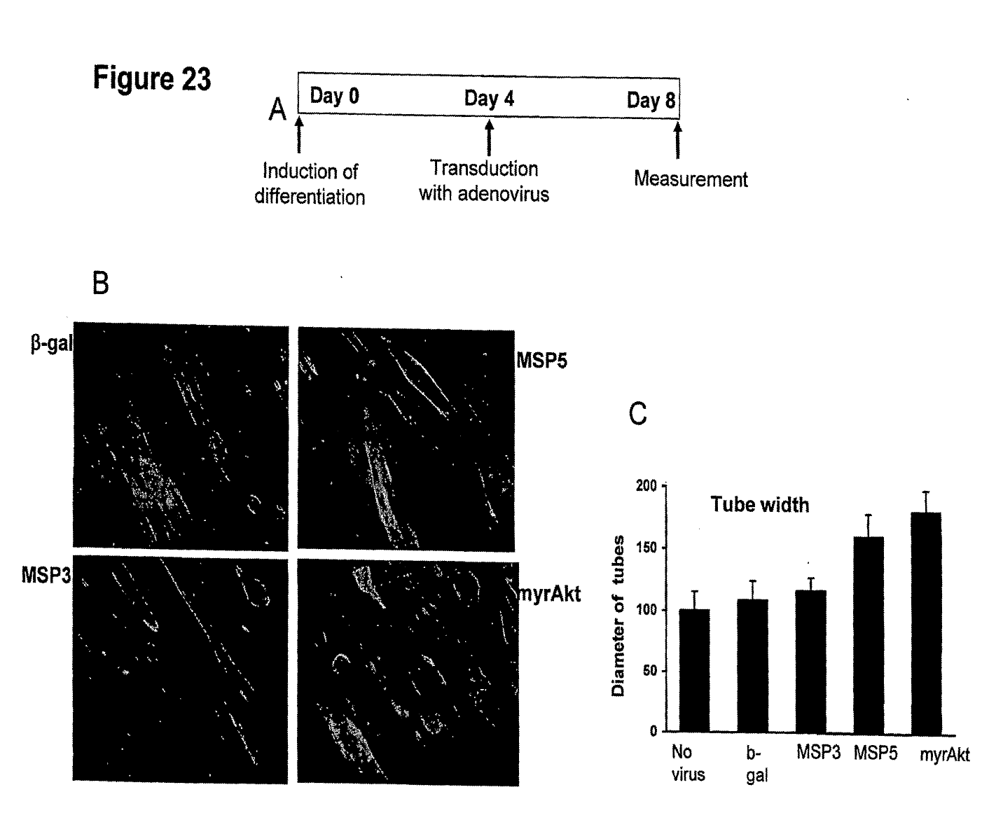

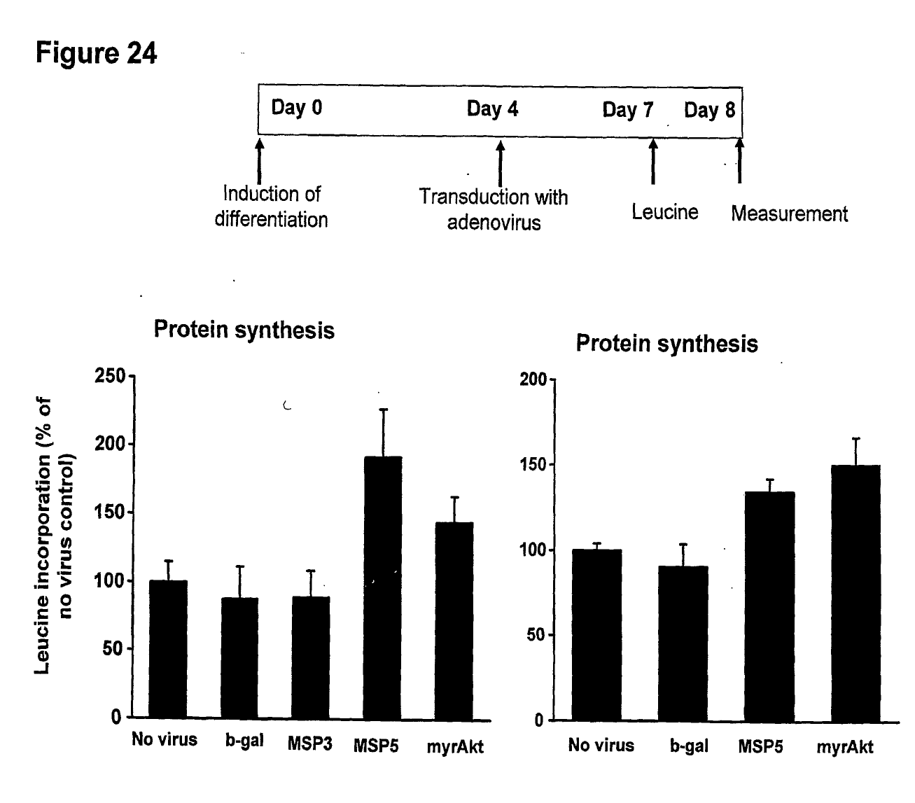

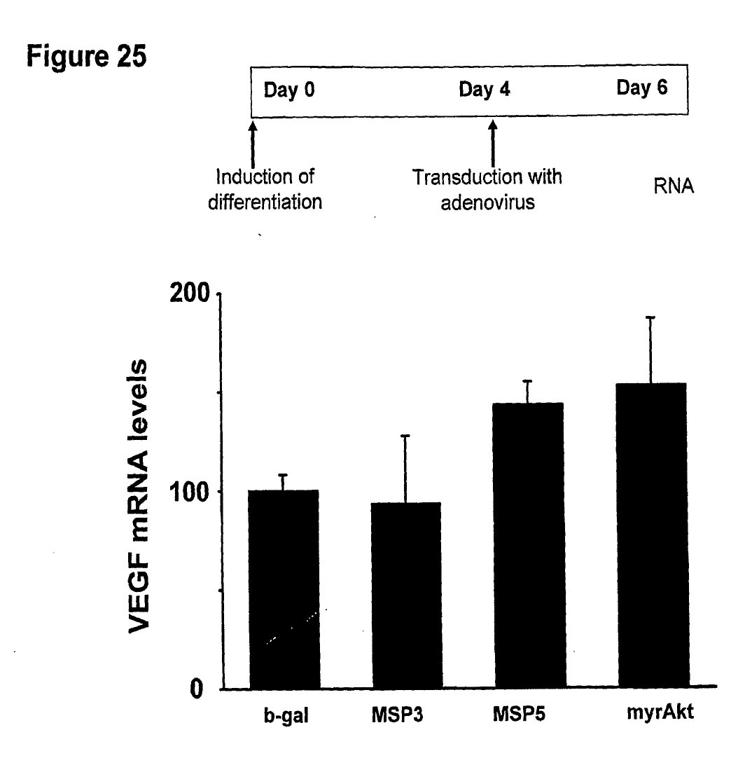

To evaluate the effect of MSP5 on growth of skeletal muscle cells in vitro, C2C12 cells four days after differentiation into myocytes were transfected with either Adv-MSP5, Adv-MSP3, adv-βGal or Adv-myrAkt and their morphology and tube diameter assessed. C2C12 cells transfected with Adv-MSP5 or adv-myrAkt are enlarged (FIG. 23B) and have increased tube diameter (FIG. 23C) compared to Adv-MSP3 or control Adv-β-gal transfected C2C12 cells. Furthermore, by assessing ³H-leucine incorporation as a measure of protein synthesis and myofibril growth, protein synthesis is increased in Adv-MSP5 and Adv-myrAkt transfected C2C12 cells compared to Adv-MSP3 and Adv-β-gal transfected (FIG. 24). C2C12 cells transfected with Adv-MSP5 or Adv-myrAkt promoted VEGF expression, an angiogenic factor, which was not observed in Adv-MSP3 or Adv-β-gal transfected cells (FIG. 25).

In summary, MSP5 functions as a muscle hypertrophy factor or a myogenic factor, and promotes hypertrophy in skeletal muscle. MSP5 also stimulates angiogenesis in ischemic limb, and increases expression of VEGF in C2C12 cells. The revascularization of Adv-MSP5 transduced ischemic limb may be the consequence of increased muscle growth and increased secretion of VEGF.

### Example 7

### Insulin-Like 6 (Ins16) Promotes Muscle Regeneration

Insulin-like 6 was also identified to be differentially expressed in response to expression of muscle related transgenes and muscle growth. Insulin-like 6 (Ins16) belongs to the relaxin family, and corresponds to GenBank Accession No. NM_{—}007179 (SEQ ID NO:20)

An increase in satellite proliferation was observed 2, 4, 6, 8 and 10 weeks (w) after activation of Akt1 in the skeletal muscle of transgenic mice that have inducible expression of a muscle-related protein, for example in mice expressing constitutively active, which was not observed in control mice (FIG. 11), as determined by a centralized nuclei in muscle cells which indicate progenitor cell recruitment as myofibrils grow. Satellite cell proliferation at 2 weeks after transgene activation is shown immunohistochemistry of BrdU incorporation into DNA which was evident in muscle histological sections from mice with induced Akt but not in control mice (cont). The cells incorporating the BrdU are multinucleated and are located around the myocyte indicating the myofibril is recruiting the satellite cells (data not shown). Further analysis by immunostaining for an activated satellite marker (myo-D), double (homozygous) Akt transgenice mice show increased myoD positive satellite cells, which are not detected in muscle from control mice (data not shown). Approximately 2-4 MyoD-positive satellite cells were seen per cross-section of gastrocnemial muscle in MyoMice, but no MyoD cells were detected in the muscle from the control mice (data not shown).

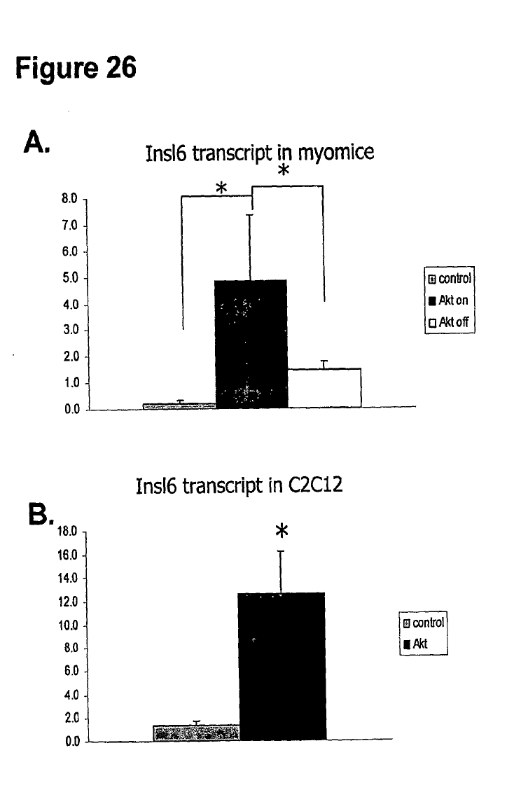

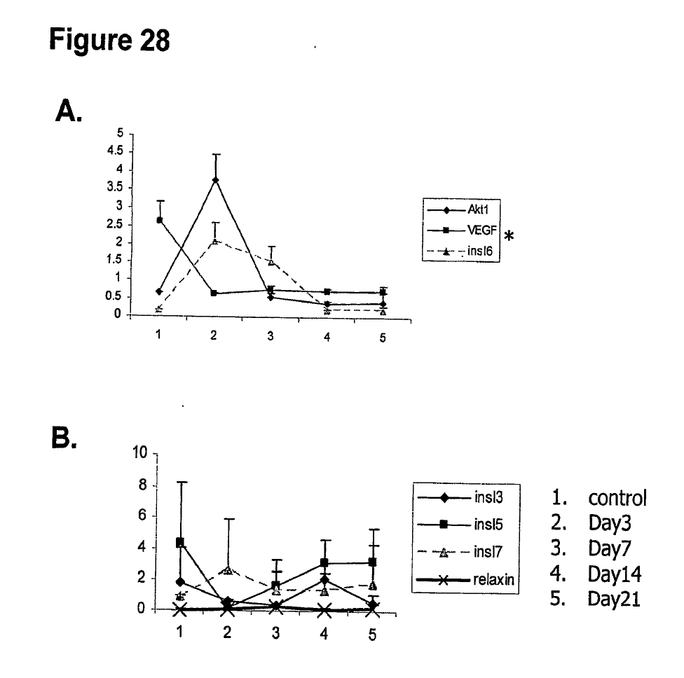

Using either transgenic mice with inducible expression of Akt, or C2C12 cells transfected with adenovirus expressing Adv-Akt, Ins16 is significantly upregulated, approximately 9-fold, in mice after induction of Akt and also upregulated in C2C12 cells expressing Adv-Akt (FIG. 26), indicating insulin-like 6 is regulated by Akt in muscle both in vitro and in vivo. Interestingly, other relaxin family members, including Ins13, Ins15, relaxin and ins17 are not regulated by Akt (FIG. 27). Furthermore, Insulin-like 6 transcript is dramatically upregulated 24-fold in transgenic mice 2 weeks after induction of Akt expression and 10-fold in C2C12 cells following transduction with Adeno-myrAkt1 (FIG. 26). In a separate experiment, Ins16 expression was analyzed in a model of muscle regeneration, where administration of cardiotoxin to tibialis anterius (TA) muscle stimulated muscle regeneration and repair. Both Akt and Ins16 transcript are upregulated during muscle regeneration following cardiotoxin administration to tibialis anterius (TA) muscle, whereas VEGF is downregulated and other members of the relaxin family (as illustrated in FIG. 41) such as Ins13, Ins15, relaxin, Ins17 (relaxin 3) are not regulated in cardiotoxin-injured mouse muscle (FIG. 28).

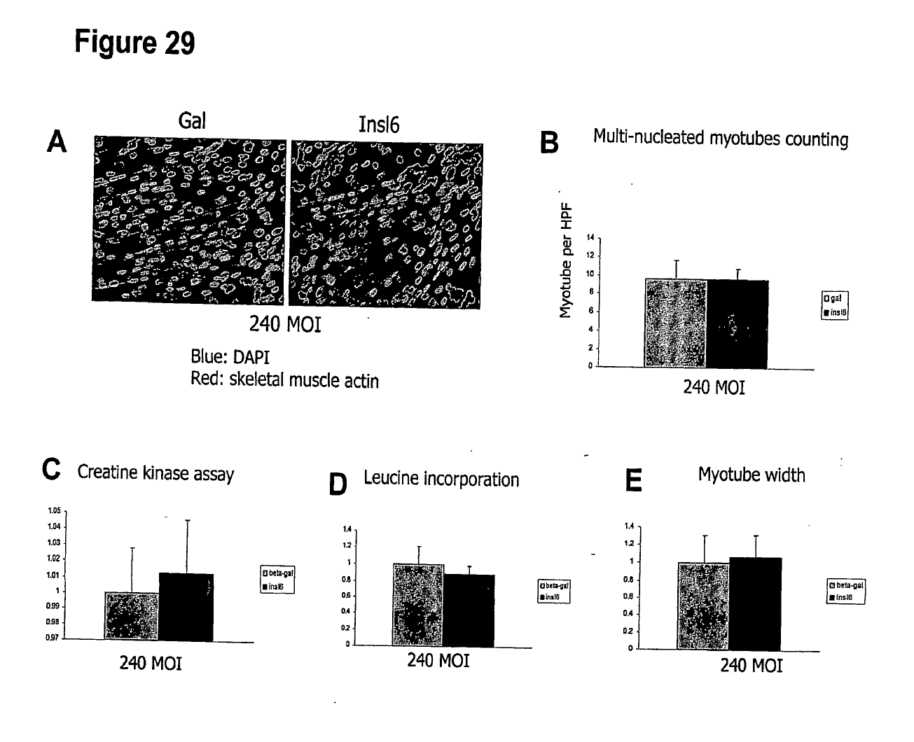

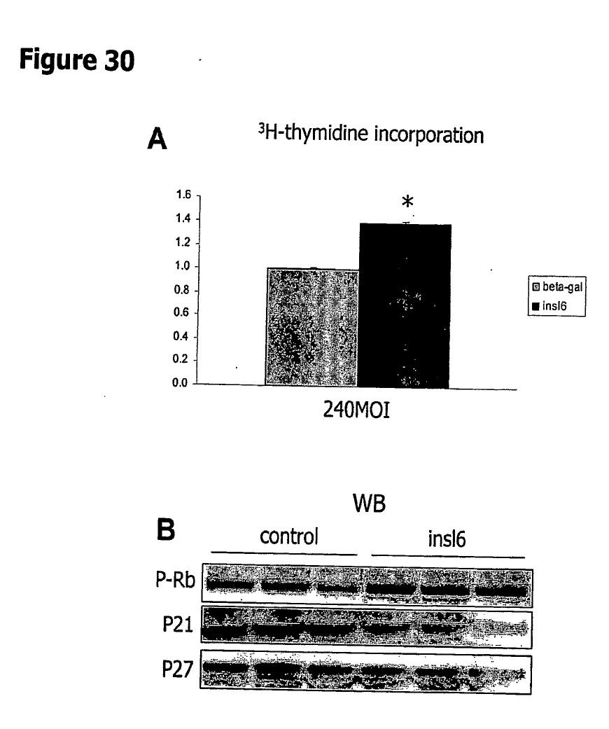

To investigate the functional role of Ins16, the inventors generated adenovirus expressing Ins16 and assessed its effect on C2C12 cells in vitro. FIG. 36 shows that C2C12 cells transfected with Adv-Ins16 or Adv-βGal at 240 MOI (multiplicities of infection), no change in morphology such as myofibril hypertrophy or differentiation of C2C12 cells occurred (FIGS. 29A and 29E), nor was there an increase in number of myotybules or change in creatine kinase expression or Leucine incorporation (FIG. 29B-E) compared to Adv-β-gal transfected C2C12 cells. Interestingly, these results with Ins16 are in contrast to what was observed for MSP5 (in Example 6 above). In additional experiments, Adv-Ins16 stimulated the proliferation of satellite cells in skeletal muscle cells, as shown by increased thymidine (3H-thymidine) incorporation in skeletal muscle (FIG. 30A), which was accompanied by increase in retinoblastoma (Rb) protein and phosphorylated Rb (p-Rb) (FIG. 30B).

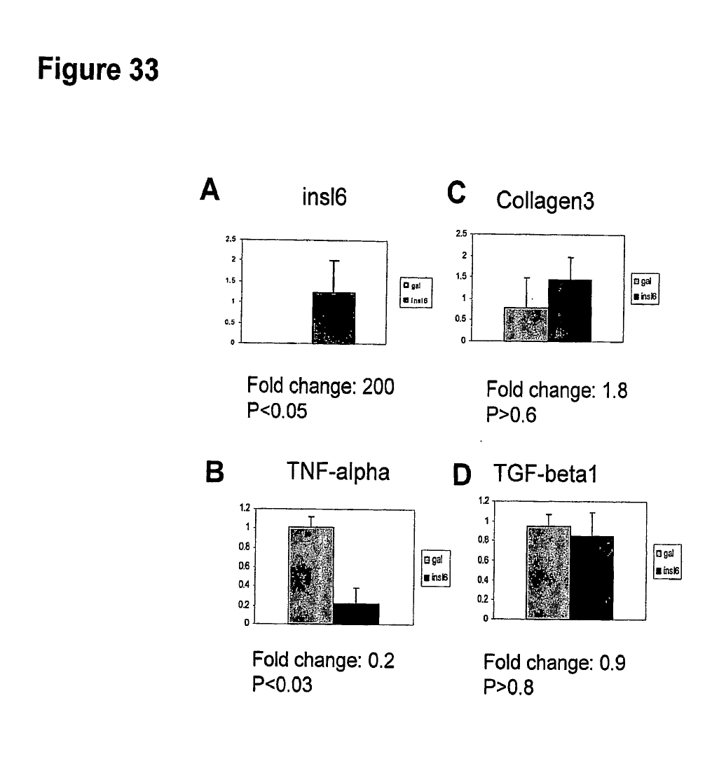

In the in vivo model of muscle regeneration using intramuscular injection of cardiotoxin (CTX) Adv-Ins16 facilitates TA muscle regeneration after cardiotoxin (CTX) injury (FIGS. 31 and 32) compared to Adv-βGal control injected mice. Improved regeneration is most notable at 7 and 14 days in histological sections (also shown in FIG. 32). Furthermore, Ins16 was also discovered to function as an anti-inflammatory factor, based on its ability to decrease the number of inflammatory cells after intramuscular administration of cardiotoxin (CTX) (FIGS. 31 and 32). At 7 days Ins16 overexpression repressed creatine kinase release into sera (lower left panel) which was not observed at 14 days (lower right panel). Quantitative analysis of transcript levels of cardiotoxin mediated muscle degeneration indicates that Ins16 mediates changes in some transcript levels, as Adv-Ins16 transfected muscle, Ins16 is increased 200-fold (p<0.05), and Adv-Ins16 expression reduces TNFα (0.2 fold, p<0.03) and TNFβ1 (0.9 fold, p>0.8), and increases the expression of collagen 3 (1.8 fold, p>0.6) (FIG. 33).

TABLE 3
Summary of Sequence listings and corresponding SEQ ID NOS:

| | | | SEQUENCE (gene |
|---|---|---|---|
| SEQ ID NO: | Clone | GenRef ID | name) |
| SEQ ID NO: 1 | MSP3 - long form (nucleotide) | NM_026754 | 1110017I16Rik |
| SEQ ID NO: 2 | MSP3 - short form (nucleotide) | NM_026754 | 1110017I16Rik |
| SEQ ID NO: 3 | MSP3 aa - mouse | NP_081030 | 1110017I16Rik |
| SEQ ID NO: 4 | MSP3 aa - rat | XP_001066258 | 1110017I16Rik |
| SEQ ID NO: 5 | MSP3 aa - human | NP_660357 | 1110017I16Rik |
| SEQ ID NO: 6 | MSP3 forward primer 1 | N/A | N/A |
| SEQ ID NO: 7 | MSP3 reverse primer 1 | N/A | N/A |
| SEQ ID NO: 8 | MSP3 forward primer 2 | N/A | N/A |
| SEQ ID NO: 9 | MSP3 reverse primer 1 | N/A | N/A |
| SEQ ID NO: 10 | MSP 3 forward primer 3 (detect both | N/A | N/A |
| | short and long forms) | | |
| SEQ ID NO: 11 | MSP 3 reverse primer 3 (detect both | N/A | N/A |
| | short and long forms) | | |
| SEQ ID NO: 12 | MSP 5 (clone 5) - nucleotide | AK005465/NM_024237 | 1600015H20Rik |
| SEQ ID NO: 13 | MSP 5 (clone 5) - amino acid - mouse | XP_001081124 | 1600015H20Rik |
| SEQ ID NO: 14 | MSP 5 (clone 2) - amino acid - rat | NP_077199 | 1600015H20Rik |
| SEQ ID NO: 15 | MSP 5 (clone 2) - amino acid - human | NP_694946 | 1600015H20Rik |
| SEQ ID NO: 16 | MSP1 (clone 9) | BC52844 | 2160028F08Rik |
| SEQ ID NO: 17 | MSP2 (clone 8) | AK009779 | 2310043I08Rik |
| SEQ ID NO: 18 | MSP4 (clone 3) | BC025830/AK028883 | 4732466D17Rik |
| SEQ ID NO: 19 | MSP6 (FGF21) | NM-019113 | FGF21 |
| SEQ ID NO: 20 | Insl6 (nucleotide) | NM_007179/ | INSL6 |
| | | AF_156094 | |
| SEQ ID NO: 21 | insl6 (amino acid) | NP_009110 | INSL6 |

### REFERENCES

1. Lecker S H, Solomon V, Mitch W E, Goldberg A L. Muscle protein breakdown and the critical role of the ubiquitin-proteasome pathway in normal and disease states. J. Nutr. 1999; 129:227S-237S.
2. Franzini-Armstrong C, Fischman D A. Morphogenesis of skeletal muscle fibers. In: Engel A G, Franzini-Armstrong C, editors. Myology: Basic and Clinical, 2nd ed. New York, N.Y.: McGraw-Hill, Inc.; 1994. p. 74-96.
3. Degens H, Turek Z, Hoofd L J C, Van't H of M A, Binkhorst R A. The relationship between capillarisation and fibre types during compensatory hypertrophy of the plantaris muscle in the rat. J. Anat. 1992; 180:455-463.
4. Degens H, Veerkamp J H, van Moerkerk H T B, Turek Z, Hoofd L J C, Binkhorst R A. Metabolic capacity, fibre type area and capillarization of rat plantaris muscle. Effects of age, overload and training and relationship with fatigue resistance. Int. J. Biochem. 1993; 25:1141-1148.
5. Ingjer F. Capillary supply and mitochondrial content of different skeletal muscle fiber types in untrained and endurance-trained men. A histochemical and ultrastructural study. Eur. J. Appl. Physiol. Occup. Physiol. 1979; 40: 197-209.
6. Kano Y, Shimegi S; Masuda K, Ohmori H, Katsuta S. Morphological adaptation of capillary network in compensatory hypertrophied rat plantaris muscle. Eur. J. Appl. Physiol. Occup. Physiol. 1997; 75:97-101.
7. Carpenter S, Karpati G. Necrosis of capillaries in denervation atrophy of human skeletal muscle. Muscle Nerve 1982; 5:250-254.
8. Hudlicka O. The response of muscle to enhanced and reduced activity. In: Harris J B, Turnbull D M, editors. Bailliere's Clinical Endocrinology and Metabolism. Toronto: Bailliere-Tindal; 1990. p. 417-439.
9. Plyley M J, Olmstead B J, Noble E G. Time course of changes in capillarization in hypertrophied rat plantaris muscle. J. Appl. Physiol. 1998; 84:902-907.
10. Musaro A, McCullagh K, Paul A, Houghton L, Dobrowolny G, Molinaro M, Barton E R, Sweeney H L, Rosenthal N. Localized Igf-1 transgene expression sustains hypertrophy and regeneration in senescent skeletal muscle. Nat. Genet. 2001; 27:195-200.
11. Sutrave P, Kelly A M, Hughes S H. ski can cause selective growth of skeletal muscle in transgenic mice. Genes Dev. 1990; 4:1462-1472.
12. Lai K M, Gonzalez M, Poueymirou W T, Kline W O, Na E, Zlotchenko E, Stitt T N, Economides A N, Yancopoulos G D, Glass D J. Conditional activation of akt in adult skeletal muscle induces rapid hypertrophy. Mol. Cell. Biol. 2004; 24:9295-9304.
13. Reaven G M. Pathophysiology of insulin resistance in human disease. Physiol. Rev. 1995; 75:473-486.
14. Mueckler M. Insulin resistance and the disruption of Glut4 trafficking in skeletal muscle. J. Clin. Invest. 2001; 107(10):1211-1213.
15. Kadowaki T. Insights into insulin resistance and type 2 diabetes from knockout mouse models. J. Clin. Invest. 2000; 106:459-465.
16. Lillioja S, Mott D M, Spraul M, Ferraro R, Foley J E, Ravussin E, Knowler W C, Bennett P H, Bogardus C. Insulin resistance and insulin secretory dysfunction as precursors of non-insulin-dependent diabetes mellitus. Prospective studies of Pima Indians. N. Engl. J. Med. 1993; 329: 1988-1992.
17. Martin B C, Warram J H, Krolewski A S, Bergman R N, Soeldner J S, Kahn C R. Role of glucose and insulin resistance in development of type 2 diabetes mellitus: results of a 25-year follow-up study. Lancet 1992; 340:925-929.
18. Knowler W C, Barrett-Connor E, Fowler S E, Hamman R F, Lachin J M, Walker E A, Nathan D M. Reduction in the incidence of type 2 diabetes with lifestyle intervention or metformin. N. Engl. J. Med. 2002; 346:393-403.
19. Datta S R, Brunet A, Greenberg M E. Cellular survival: a play in three Akts. Genes Dev. 1999; 13:2905-2927.
20. Chen W S, Xu P Z, Gottlob K, Chen M L, Sokol K, Shiyanova T, Roninson I, Weng W, Suzuki R, Tobe K, Kadowaki T, Hay N. Growth retardation and increased apoptosis in mice with homozygous disruption of the Akt1 gene. Genes Dev. 2001; 15:2203-2208.
21. Verdu J, Buratovich M A, Wilder E L, Birnbaum M J. Cell-autonomous regulation of cell and organ growth in Drosophila by Akt/PKB. Nat. Cell Biol. 1999; 1:500-506.
22. Ueki K, Yamamoto-Honda R, Kaburagi Y, Yamauchi T, Tobe K, Burgering B M, Coffer P J, Komuro I, Akanuma Y, Yazaki Y, Kadowaki T. Potential role of protein kinase B in insulin-induced glucose transport, glycogen synthesis, and protein synthesis. J. Biol. Chem. 1998; 273:5315-5322.
23. Rommel C, Clarke B A, Zimmerman S, Nunez L, Rossman R, Reid K, Moelling K, Yancopoulos G D, Glass D J. Differentiation stage-specific inhibition of the Raf-MEK-ERK pathway by Akt. Science 1999; 286:1738-1741.
24. Bodine S C, Stitt T N, Gonzalez M, Kline W O, Stover G L, Bauerlein R, Zlotchenko E, Scrimgeour A, Lawrence J C, Glass D J, Yancopoulos G D. Akt/mTOR pathway is a crucial regulator of skeletal muscle hypertrophy and can prevent muscle atrophy in vivo. Nat. Cell Biol. 2001; 3:1014-1019.
25. Hixon M L, Muro-Cacho C, Wagner M W, Obejero-Paz C, Millie E, Fujio Y, Kureishi Y, Hassold T, Walsh K, Gualberto A. Akt1/PKB upregulation leads to vascular smooth muscle cell hypertrophy and polyploidization. J. Clin. Invest. 2000; 106:1011-1020.
26. Ushio-Fukai M, Alexander R W, Akers M, Yin Q, Fujio Y, Walsh K, Griendling K K. Reactive oxygen species mediate the activation of Akt/protein kinase B by angiotensin II in vascular smooth muscle cells. J. Biol. Chem. 1999; 274:22699-22704.
27. Shiojima I, Sato K, Izumiya Y, Schiekofer S, Ito M, Liao R, Colucci W, Walsh K. Disruption of coordinated cardiac hypertrophy and angiogenesis contributes to the transition to heart failure. J. Clin. Invest. 2005; 115:2108-2118.
28. Takahashi A, Kureishi Y, Yang J, Luo Z, Guo K, Mukhopadhyay D, Ivashchenko Y, Branellec D, Walsh K. Myogenic Akt signaling regulates blood vessel recruitment during myofiber growth. Mol. Cell. Biol. 2002; 22:4803-4814.

All references described herein are incorporated herein by reference.

1. A method of modulating a metabolic function in a subject, the method comprising administering to a subject an effective amount of a pharmaceutical composition comprising an agent that affects the activity and/or expression of metabolic regulator, wherein the metabolic regulator is selected from the group of MSP1, MPS2, MPS3, MPS4, MPS5 or Ins16 or homologues thereof, or their expression product or functional fragments thereof, and changing the expression of the metabolic regulator or its level in a cell modulates at least one metabolic function.

2. The method of claim 1, wherein the metabolic function is selected from the group of: muscle mass, fat mass, angiogenesis, body weight, insulin and/or glucose sensitivity, of combinations thereof.

3. The method of claim 1, wherein the agent is an agonist of MSP1, MPS2, MPS3, MPS4, MPS5 or Ins16.

4. The method of claim 1, wherein the agent is an antagonist of MSP1, MPS2, MPS3, MPS4, MPS5 or Ins16.

5. The method of claim 1, wherein the agent is a small molecule, nucleic acid, nucleic acid analogue, peptide, protein, ribosome, antibody, inhibitory nucleic acid, antisense oligonucleotides, RNAi, shRNA, siRNA, miRNA, and variants and fragments thereof.

6. The method of claims 1 or 3, wherein the agent is the expression product of MSP1, MPS2, MPS3, MPS4, MPS5 or Ins16, or a homologue or variant thereof.

7. The method of claim 4, wherein the antagonist is a small molecule, nucleic acid, nucleic acid analogue, peptide, protein, ribosome, antibody, inhibitory nucleic acid, antisense oligonucleotides, RNAi, shRNA, siRNA, miRNA, and variants and fragments thereof

8. The method of claim 1, wherein the agent is targeted to the muscle.

9. The method of claim 1, wherein the MSP1 is encoded by nucleic acid sequence SEQ ID NO:16 or a fragment or functional variant or homologue thereof.

10. The method of claim 1, wherein the MSP2 is encoded by nucleic acid sequence SEQ ID NO: 17 or a fragment or functional variant or homologue thereof.

11. The method of claim 1, wherein the MSP3 is encoded by nucleic acid sequence SEQ ID NO: 1 or SEQ ID NO:2 or a fragment or functional variant or homologue thereof.

12. The method of claim 1, wherein the MSP4 is encoded by nucleic acid sequence SEQ ID NO:18 or a fragment or functional variant or homologue thereof.

13. The method of claim 1, wherein the MSP5 is encoded by nucleic acid sequence SEQ ID NO: 12 or a fragment or functional variant or homologue thereof.

14. The method of claim 1, wherein the Ins16 is encoded by nucleic acid sequence SEQ ID NO:20 or a fragment or functional variant or homologue thereof.

15. The method of claim 1, wherein the subject is suffering from a condition or disease selected from a group of: muscle degeneration, insulin-related disorder, muscle atrophy, obesity or blood vessel loss or combinations thereof.

16. The method of claim 15, wherein the insulin-resistant disorder is non-insulin dependent diabetes mellitus (NIDDM) or insulin-dependent diabetes (IDD) or obesity.

17. The method of claim 1, wherein the subject is suffering from angiogenesis associated with ocular neovascularization, tumor angiogenesis, arthritis, retionopathy, retinopathy of prematurity, age-related macular degeneration, diabetic related retinopathy, psorasis, capillary proliferation in atherosclerotic plaques.

18. The method of claim 1, wherein the metabolic function that is modulated is selected from the group of: insulin sensitivity and/or glucose sensitivity or body weight or angiogenesis or combinations thereof, and the agent is an agent for MSP3.

19. The method of claim 18, wherein the metabolic function is increased insulin sensitivity and/or increased glucose sensitivity or decreased body weight or increased angiogenesis or combinations thereof, and the agent is an agonist for MSP3.

20. The method of claim 18, wherein the metabolic function is decreased insulin sensitivity and/or decreased glucose sensitivity or increased body weight or decreased angiogenesis or combinations thereof, and the agent is an antagonist for MSP3.

21. The method of claim 1, wherein the metabolic function that is modulated is selected from the group of: muscle mass, hypertrophy or angiogenesis or combinations thereof, and the agent is an agent for MSP5.

22. The method of claim 21, wherein the metabolic function is increased muscle mass and/or increased hypertrophy and/or increased body weight increased angiogenesis or combinations thereof, and the agent is an agonist for MSP5.

23. The method of claim 21, wherein the metabolic function is decreased muscle mass and/or decreased hypertrophy and/or decreased body weight and/or decreased angiogenesis or combinations thereof, and the agent is an agonist for MSP5.

24. The method of claim 1, wherein the metabolic function that is modulated is selected from the group of: muscle mass or muscle regeneration or combinations thereof, and the agent is an agent for Ins16.

25. The method of claim 24, wherein the metabolic function is increased muscle mass or increased muscle regeneration or increased weight loss or combinations thereof, and the agent is an agonist for Ins16.

26. The method of claim 24, wherein the metabolic function is decreased muscle mass or decreased muscle regeneration or decreased weight loss or combinations thereof, and the agent is an antagonist for Ins16.

27.-33. (canceled)

34. The method of claim 1, wherein modulating is an increase in the metabolic function in the subject.


SEQUENCE LISTING (24 sequences)

SEQ ID NO: 1 - DNA, 417 bp, Mus musculus
atgtcctggagacgggtcattctcctgtcatctctcttggccctggtgctcctgtgtatgctacaggaggggaccagcgcttctgtggggagcaggcaggcagctgcagagggggtgcaggaaggtgtgaaacagaagattttcatgcaagaatctgatgcctccaatttcctcaagaggcgtggcaagcggtctcctaagtcccgagatgaagttaatgcggaaaacagacagaggctgcgggatgatgagctgcggagggagtattacgaggagcaaaggaacgagtttgagaacttcgtggaggaacagagagatgagcaggaagagaggacccgggaggctgtggagcagtggcgccagtggcattatgatggcctgtatccttcctacctctacaaccgccaaaacatctga

SEQ ID NO: 2 - DNA, 351 bp, Mus musculus
atgtcctggagacgggtcattctcctgtcatctctcttggccctggtgctcctgtgtagtgtgaaacagaagattttcatgcaagaatctgatgcctccaatttcctcaagaggcgtggcaagcggtctcctaagtcccgagatgaagttaatgcggaaaacagacagaggctgcgggatgatgagctgcggagggagtattacgaggagcaaaggaacgagtttgagaacttcgtggaggaacagagagatgagcaggaagagaggacccgggaggctgtggagcagtggcgccagtggcattatgatggcctgtatccttcctacctctacaaccgccaaaacatctga

SEQ ID NO: 3 - protein, 138 aa, Mus musculus
MSWRRVILLSSLLALVLLCMLQEGTSASVGSRQAAAEGVQEGVKQKIFMQESDASNFLKRRGKRSPKSRDEVNAENRQRLRDDELRREYYEEQRNEFENFVEEQRDEQEERTREAVEQWRQWHYDGLYPSYLYNRQNI

SEQ ID NO: 4 - protein, 138 aa, Rattus norvegicus
MSWRQVILLSSLSALVLLCMLQEGTSASVGSRQAAGEEVQEGMKQKIFMQESDASNFLKRRGKRSPKSRDEVTAENRQRLRDDELRREYYEEQRNEFENFVEEQRDEQEERTREAVEQWRQWHYDGLYPSYLYNRQNI

SEQ ID NO: 5 - protein, 138 aa, Homo sapiens
MTWRQAVLLSCFSAVVLLSMLREGTSVSVGTMQMAGEEASEDAKQKIFMQESDASNFLKRRGKRSPKSRDEVNVENRQKLRVDELRREYYEEQRNEFENFVEEQNDEQEERSREAVEQWRQWHYDGLHPSYLYNRHHT

SEQ ID NO: 6 - DNA, 21 bp, Artificial Sequence
  Description of Artificial Sequence Synthetic primer
atgtcctggagacgggtcatt

SEQ ID NO: 7 - DNA, 21 bp, Artificial Sequence
  Description of Artificial Sequence Synthetic primer
tacaaccgccaaaacatctga

SEQ ID NO: 8 - DNA, 23 bp, Artificial Sequence
  Description of Artificial Sequence Synthetic primer
cctgtcatctctcttggccctgg

SEQ ID NO: 9 - DNA, 20 bp, Artificial Sequence
  Description of Artificial Sequence Synthetic primer
acgaggagcaaaggaacgag

SEQ ID NO: 10 - DNA, 22 bp, Artificial Sequence
  Description of Artificial Sequence Synthetic primer
gtcccgagatgaagttaatgcg

SEQ ID NO: 11 - DNA, 21 bp, Artificial Sequence
  Description of Artificial Sequence Synthetic primer
acgaggagcaaaggaacgagt

SEQ ID NO: 12 - DNA, 1630 bp, Mus musculus
ggcggactagtggccttggaagctgcaagggacccgcgcactgcggccagagaacagcgacctgccttccccagcagcaaggctgtgagtggcaagatgggaccgggctcacagcgcgcgcttttcctcctcttgctgctcctcgccagccccggagcgcgggctttccagagctgtctcaacaaacagcagctcctcaccaccatccgccagctgcagcagctgctgaaaggccaggagacccgcttcaccgaagcgatccgaaacatgaagagccggctggctgcactgcaaaacacggttaacaaaatgaccccggatgcaccaccagtttcctgcccagctctggaggcccctccggatggcaagaagtttggaagcaagtacttagtggaccatgaagtctattttacctgcaaccctgggttccagctggttgggcccagcagcgtggtgtgtcttgctaatggtagctggacaggagagcagccccgctgcagagatatcagtgaatgctccagccagccttgtcacaatggagggacgtgtgtggaaggcatcaaccactacagatgcatctgtcctccaggaaaaactgggaatggctgtcagcatcagacccaggctgcggccccagatggcggcgaggcggtgacccgccttcagccgcgcacccgctgcgcgcaggtggagcgggaacagcattgcagctgcgaggcgggattccacctgagcagcaccacgggcggccacagcgtctgccaggatgtgaatgagtgtgagatctatgggcagaaaggacgcccccggctctgcatgcatgcctgtgtgaacacccctggttcctaccgatgtacctgcccgagtggataccggatcctggctgatgggaagagctgtgaggatgttgatgagtgtgcaggcccacaacacatgtgcccccgggggaccacatgcatcaacactggaggaggcttccagtgtgtcaaccctgagtgtcctgagggcagcggcaatataagctacgtgaagacatctccctttcagtgcgagcgaaacccttgtcccatggacagcaggccatgccgccacctgcccaagaccatctccttccattacctctctctcccttccaagttgaagacacccatcacgctcttccgcatggccacagcctcaattcctggccatcctgggcccaacagcctgcgctttgggatcgtgggtgggaacagccgtggccacttcgtaatgcagcgctcagaccggcagacaggagagctcatccttacacagaccctggaggggcctcagactctggaggttgacgtagacatgtcggaatacctggagcgctccttccaggccaaccatgtatccaaggtcaccatctttgtttctcgctatgacttctgaggatgccatggcagtcgggaggctggggtttgagatctgggctgatttcacttccccaaggacacattgtgggcaagagctgtggtggtcattcttttcttggtcatgttcttacctactctgcttgttcattcaggcttctagatcaatgctgtgttctgacccaggggctgccacagaagggaagccacaaacaaatgctgtatctaccatc

SEQ ID NO: 13 - protein, 567 aa, Rattus norvegicus
MTTGPALSSSEEGLMSEMPPAWCHTARADHSTPGTSPEMIPDTQGHWMPLRSRGLGALPGAFQAPQPLLSRLRHRLAPPRIVGTVSCRRLRTIVALGAARDSCTVGTQGRPGNSDLPAFPTSQVVRGKMGPGSQRTLVLLLLLLASPGARAFQSCLNKQQLLTTVRQLQQLLKGQETRFTEGIRNMKSRLTALQNTVSKMTPDAPPVSCPALDAPPNGKKFGSKYLVDHEVHFTCNPGFQLVGPSSVVCLANGTWTGEQPRCRDTSECSSQPCHNGGTCVEGVHHYRCICPPGKTGNRCQHQTQAAAPGGVAGDSAYSRAPRCAQVEREQHCSCEAGFHLSGTAGGHSVCQDVNECEIYGQEGRPRLCMHACVNTPGSYRCTCPSGYRILADGKSCEDVDECAGPQHMCPRGTTCINTGGGFQCVNPECPEGSGNISYVKTSPFQCERNPCPMDSRPCRHLPKTISFHYLSLPSNLKTPITLFRMATASVPGHPGPNSLRFGIVGGNSRGHFVMQRSDRQTGELILIQTLEGPQTLEVEVDMSEYLERSFQASHVSKVTIFVSRYDF

SEQ ID NO: 14 - protein, 439 aa, Mus musculus
MGPGSQRALFLLLLLLASPGARAFQSCLNKQQLLTTIRQLQQLLKGQETRFTEAIRNMKSRLAALQNTVNKMTPDAPPVSCPALEAPPDGKKFGSKYLVDHEVYFTCNPGFQLVGPSSVVCLANGSWTGEQPRCRDISECSSQPCHNGGTCVEGINHYRCICPPGKTGNGCQHQTQAAAPDGGEAVTRLQPRTRCAQVEREQHCSCEAGFHLSSTTGGHSVCQDVNECEIYGQKGRPRLCMHACVNTPGSYRCTCPSGYRILADGKSCEDVDECAGPQHMCPRGTTCINTGGGFQCVNPECPEGSGNISYVKTSPFQCERNPCPMDSRPCRHLPKTISFHYLSLPSKLKTPITLFRMATASIPGHPGPNSLRFGIVGGNSRGHFVMQRSDRQTGELILTQTLEGPQTLEVDVDMSEYLERSFQANHVSKVTIFVSRYDF

SEQ ID NO: 15 - protein, 439 aa, Homo sapiens
MVPSSPRALFLLLLILACPEPRASQNCLSKQQLLSAIRQLQQLLKGQETRFAEGIRHMKSRLAALQNSVGRVGPDALPVSCPALNTPADGRKFGSKYLVDHEVHFTCNPGFRLVGPSSMVCLPNGTWTGEQPHCRGISECSSQPCQNGGTCVEGVNQYRCICPPGRTGNRCQHQAQTAAPEGSVAGDSAFSRAPRCAQVERAQHCSCEAGFHLSGAAGDSVCQDVNECELYGQEGRPRLCMHACVNTPGSYRCTCPGGYRTLADGKSCEDVDECVGLQPVCPQGTTCINTGGSFQCVSPECPEGSGNVSYVKTSPFQCERNPCPMDSRPCRHLPKTISFHYLSLPSNLKTPITLFRMATASAPGRAGPNSLRFGIVGGNSRGHFVMQRSDRQTGDLILVQNLEGPQTLEVDVDMSEYLDRSFQANHVSKVTIFVSPYDF

SEQ ID NO: 16 - DNA, 2685 bp, Mus musculus
aagaggtaatcctgcagctgtgccatctgcgttaaactgctgcgtggatcgtgccagttcaccgtggagggagagatgctcatcgagccaaattgatcattgcagccgcagggcagtgacatctgtctctaagtcctccctaggagcgcgacccgcactgtctccttccaggagcccgtcatttcctcgacttttgagaggtgtctctccccagcccgaccgtccagatgcgtttttgcctcttctcatttgccctcatcattctgaactgtatggattacagccagtgccaaggcaaccgatggagacgcaataagcgagctagttatgtatcaaatcccatttgcaagggttgtttgtcttgttcgaaggacaatggttgcagccgatgtcaacagaagttgttctttttccttcgaagagaaggaatgcgtcagtatggagagtgcctgcattcctgcccatcagggtattatggacaccgagccccagatatgaacagatgtgcacgatgcagaatagaaaactgtgattcttgctttagcaaagacttttgtacgaagtgcaaagtaggcttttatttgcatagaggccgctgctttgatgaatgtccagatggttttgcaccgttagatgagactatggaatgtgtagaaggttgtgaagttggtcattggagcgaatgaggaacgtgtagcagaaacaaccgcacgtgtggatttaaatggggtctggaaaccagaacacggcagattgttaaaaagccagcaaaagacacaataccatgtccgaccattgcggagtccaggagatgcaagatggccatgaggcactgtccaggaggaaagagaacaccaaaggcaaaagagaagagaaacaagaagaagaggcggaagctgattgagagagcccaagagcagcacagcgtcttcctcgctacagacagagtgaaccaataaaatacaagaaatagctggggcattttgaggttttctgttttgtttatgttgttgttttgcaaaagtgcacaaagctactctccagtccacactggtggacagcattcctgatcctctgaccagtatccattttcagtaatgctgcagagggaggtgcccaagcatggactcagcgttatttatgctttgattggaatctggggcctgtgatggcaggagcttgttgagctgagtcagcgggagctgatgcatctgtactcttgtgatgagcacagtgtgtcataagaacctgtccctggcacggtggacccacaggaggcacaaggctgtagatcaccaccagagaatgcacctgtgcctattttgatggatggcaatgctaagcaagcaagcactgttcacttgtgactttcatttctcacactgtgcactgtcaaagacaaatgtgcatggaaaaatgtttagtgtcacctcatggcgttctcagcatcagtgaccttcaaacggtcctacaatgagactgtgttctagctaggggtatgctgtggaaattcctgccacatttcatcttagtgctaacatgtacagattctgctgcgctacattcaaagctcattactgtatatttatgctttctctgtgtaacaagttatacctgataagatgtcactttgtttctagtggttcttaaccatggtctggtacatggctattctagttttggaaattaacaagtgttttgttgcctcttgttttcttttgttcctatcatttttggcgggggtgggatgggcttgattctaaccgtaagtataggataagctagttttgtatatagagtcaaatgactgatgtcagaggatcagtgctgatagaacttccccagttcatgtcaccatacgcacagagagaaagcagcatgaggcatcttgccatcagaagccaaatttcttttgagtcccaaaattgatgacttatgaaatatagctgaaaacaagatttgggtgtagttacttgtatttattatacaatttccaattacattttttttcaaactcaaaataacccatgactttgagtgataggtcacttggcaatgttcttgaattactggggaagctgttgtcactaagataatgagagagaaaatagaatggcttcgcccaagtgagagccacatcttacatttctctgttgaatcggaatcaactatattagaacagaagcctgatggaagctttctagttaacacacacaaggccatggtttcaaaaacatctttgtccccttaggtcagtttgtccttagattatgaattggcaggttctaattgcattatttccctggctgatccaggaaaaagttagaacaaaataagttgcatagttttgaggaaacatccaaagcaaggcgaagcctttccttgccttgcattggcaaaactacctctttagcatttatgttgattcagaaacatcttgctgatatgtgtagatgttttaagcttcattgtgaaaatattgatgcaagataagccatatatgaatgttgtattcaactttagggcttgaaattaatcctaaagtgttcacctctctccatgtctatttacactctgttcctatttactaagagggtaggggtctccttaatatcatacttcattgttaataagtcaatgcttgttatgtttcttggctgttgtttttgtgctaaaaaaaaaaaa

SEQ ID NO: 17 - DNA, 930 bp, Mus musculus
ggtctaaggaacatggccccggggccgtcagccacacaggggatcctgctgctgctgcctctcctgcctctgtcccaggtgacgctgggttccgcggaccgcaactgtgacccctcggatcagtgcccgccccaggcccgctggagcagcctatggcatgtggggctcatcctgctcgccatactcctgatgcttttgtgtggggtcacagccagctgtgtacgattctgctgcctcaggaagcagacgcacacccagtcacacacgccagcggcgtggcagccctgtgacgggacagtcatcccagtggacagtgatagccccgcacacagcactgtgacctcctacagttccgtgcagtacccactgggcatgcggctgcccctgtactttggagagccagaccctgactccatggtccctcccacctacagcctctatgcgtctgagctgccaccctcctacgatgaagttgtgaagatgataaaagccagggaggaagtggcagctccctctgagaaaaccaactctctgcctgaggccttggagccagagaccactggagggccccaggagccaggccccagtgcccagcggccctagtcaagcccgcttgaaaagggtgggagttctctcttctggagccattaaaccattgcctggattcctggcacttaggaccaaggagccccacctgtgtcatccttcccctgggaaggccagccatccagcacattgctgcggacgcggcctcgttgctatgcaatgcctggccagccagcctgcccctgtattggcccctcttcttcacccttcccatccatcgggggcccagcaggccctcatcccggaggagcctggatcttgggagctcaaaccccactggcatgccagccctttgaggggaaaataaagtttacgtaaacatgt

SEQ ID NO: 18 - DNA, 2304 bp, Mus musculus
ggtggcttttggggagagacatggctgagctacttgctagcttgcccgcctgggcagctgtgctccttctctttttcgctacggtctccggatccactggccctagaagcagggaaaatcggggggcgtcccggatcccttcccagttcagcgaggaggagcgtgtcgctataaaagaggcgctgaaaggtgccatccagattcccacagtgtctttcagccacgaggaatccaacaccacagcccttgctgagtttggagaatatatccgcaaagccttccctacagtgttccacagcagccttgtccaacatgaagtcgtggcaaagtatagccacctgttcaccatccaaggctcagaccccagtttgcagccctacatgctgatggctcacattgatgtggttcctgccccggaagaaggatgggaggtgcccccgttctcaggcctggaacgcaatggcttcatctatggccggggtgcgctggacaacaaaaactctgtgatggcgatcctgcatgctttggagctcctgttgatcagaaactacagccccaaaagatctttcttcattgctttgggccatgatgaggaggtgtccggggaaaagggggctcagaagatctcagcactcttacaggcaaggggtgtccagctagccttccttgtggatgaagggagctttatcttggaaggcttcattccaaacctcgagaagccagttgccatgatttcagtcactgagaagggtgcccttgacctcatgctgcaagtaaacatgactccaggccactcttcagctcccccaaaggagacaagcattggcattctttctgccgctgtcagccgactggagcagacaccaatgccgaatatgtttggaggagggccattgaagaagacaatgaagctactggcaaatgagttttccttccctatcaatatagtcttgagaaacctgtggctatttcatcccattgtgagcaggataatggagaggaaccccataacaaatgcgctggtccgaactaccacagccctcaccatgttcaatgcaggaatcaaggtgaatgtcatccctccattggctcaggctacaatcaactgccgaattcacccttcgcagacagtacatgaggtcctagaacttgtcaagaacaccgtggctgatgacagagtccagctgcatgtgttgagatcctttgaacccctgcccatcagcccctctgatgaccaggccatgggctaccagctgcttcaagagaccatacgatctgtcttcccggaagtcgacatcgtcgtccccggtatttgtattgccaatacggacacccgacactatgccaacatcaccaatggcatgtaccggttcaacccccttcccctgaaccctcaggacttcagtggtgtccatggaatcaatgagaaagtttccgttcagaactaccagaaccaggtgaagttcatctttgagttcatccaaaatgccgacacttacaaagagccagttcctcatctgcatgaactatgagctgagacttcatagttgaatgcgacagagaactgaaagaacgctaagatgagggagcagctggcacacaagatcatctgaaaacagcagtgttagatcgggcctcctacatcagggacagaagagaagttcagcaaagaccctttcttgggtcctgtcttttgttccttctacctaagccttgtccaggattccctatctcctaggcactgtgatacatcaaggccatcggtgctgatctaactagcctgtgaaataagctatgcagtagaacaagtggaaaccagaggcggtgcaatggaaaacattatgcaaatgtgaaagttgttagtcattagggaaatgcaaattttaactaccccgagaaatcactttatattcaccaggttggtcgtaatcaaaaaaaaaaaaaaatggaaaataacaaatgttggcaaggatatagagacattggaagcctcgtgcattcgctggtgggaatgtgaaatggcacagcagctgtggagtcagctgtggaaacggttggtgtttcctttgaagttaaatggagaattaccgtattaccctgcaattccacttcaaagcgtacggtcaagagaaatgaaaacaacaggtgttcaaacacctttttgttgacccaaaaatgacatcacagaacttttttaagtacaaaaacttttatgtatacaattaaaaatatatacatgtctatgctcaaagactttgtaatgtaaattgggagataaaataaatgtaataaaacaagatgttc

SEQ ID NO: 19 - DNA, 940 bp, Homo sapiens
ctgtcagctgaggatccagccgaaagaggagccaggcactcaggccacctgagtctactcacctggacaactggaatctggcaccaattctaaaccactcagcttctccgagctcacaccccggagatcacctgaggacccgagccattgatggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcctgaagccagaggctgtttactatgacatctcctctttatttattaggttatttatcttatttatttttttatttttcttacttgagataataaagagttccagaggagaaaaaaaaaaaaaaaaaaaaaaaaaaaaaaaaaaaaaaaaaaaaaaaaaaaaaaa

SEQ ID NO: 20 - DNA, 711 bp, Homo sapiens
gcctggggtcacagggatgccgcggctcctccgcttgtccctgctgtggcttggactcctgctggttcggttttctcgtgaactgagcgacatcagcagtgccaggaagctgtgcggcaggtacttggtgaaagaaatagaaaaactctgcggccatgccaactggagccagttccgtttcgaggaggaaacccctttctcacggttgattgcacaggcctcggagaaggtcgaagcctacagcccataccagttcgaaagcccgcaaaccgcttccccggcccggggaagaggcacaaacccagtgtctacttcttgggaagaagcagtaaacagttgggaaatgcagtcactacctgagtataaggataaaaagggatattcaccccttggtaagacaagagaattttcttcatcacataatatcaatgtatatattcatgagaatgcaaaatttcagaagaaacgtagaaacaaaattaaaaccttaagcaatttgttttgggggcatcatccccaaagaaaacgcagaggatattcagaaaagtgttgtcttacaggatgtacaaaagaagaacttagcattgcatgtcttccatatattgattttaaaaggctaaaggaaaaaagatcatcacttgtaactaagatatactaaccatcttagaattttttctaacctaataaaagcttaatacatttatttaaaaa

SEQ ID NO: 21 - protein, 213 aa, Homo sapiens
MPRLLRLSLLWLGLLLVRFSRELSDISSARKLCGRYLVKEIEKLCGHANWSQFRFEEETPFSRLIAQASEKVEAYSPYQFESPQTASPARGRGTNPVSTSWEEAVNSWEMQSLPEYKDKKGYSPLGKTREFSSSHNINVYIHENAKFQKKRRNKIKTLSNLFWGHHPQRKRRGYSEKCCLTGCTKEELSIACLPYIDFKRLKEKRSSLVTKIY

SEQ ID NO: 22 - DNA, 3071 bp, Mus musculus
aggcgatcctgcatgctttggagctcctgttgatcagaaactacagccccaaaagatctttcttcattgctttgggccatgatgaggaggtgtccggggaaaagggggctcagaagatctcagcactcttacaggcaaggggtgtccagctagccttccttgtggatgaagggagctttatcttggaaggcttcattccaaacctcgagaagccagttgccatgatttcagtcactgagaagggtgcccttgacctcatgctgcaagtaaacatgactccaggccactcttcagctcccccaaaggagacaagcattggcattctttctgccgctgtcagccgactggagcagacaccaatgccgaatatgtttggaggagggccattgaagaagacaatgaagctactggcaaatgagttttccttccctatcaatatagtcttgagaaacctgtggctatttcatcccattgtgagcaggataatggagaggaaccccataacaaatgcgctggtccgaactaccacagccctcaccatgttcaatgcaggaatcaaggtgaatgtcatccctccattggctcaggctacaatcaactgccgaattcacccttcgcagacagtacatgaggtcctagaacttgtcaagaacaccgtggctgatgacagagtccagctgcatgtgttgagatcctttgaacccctgcccatcagcccctctgatgaccaggccatgggctaccagctgcttcaagagaccatacgatctgtcttcccggaagtcgacatcgtggtccccggtatttgtattgccaatacggacacccgacactatgccaacatcaccaatggcatgtaccggttcaacccccttcccctgaaccctcaggacttcagtggtgtccatggaatcaatgagaaagtttccgttcagaactaccagaaccaggtgaagttcatctttgagttcatccaaaatgccgacacttacaaagagccagttcctcatctgcatgaactatgaggcaaggatccagctgggtgagggatgcccagcactgggctcaggactaacctaaagggagagagagctggtattaatgaagggtttggtggaaaccatcctgaacagagttcacctgcctgacttccctcctccactcaccagggattgaggtcccaggaggaaagggagatgaacagaatctgccctgctgtcctcctgacatctgcgttcttcctctgagtggcaactactgccttctggtgtgtcctgcctgttggtctgtctcacgccgattattaactattccactttcccagcctgtcttaacaaatacagattttggaaagacctcaagcaggtttaaatactagagtattgacttcaggtacctgaggctggactgtcttctagaatgccctttctcctgtcttctctccgactcgggagctgccccttggcctctctcttcctgcctctgcttccttcctccagtctccagctcattccacatctggagttaagactcaaacttagaagctcccatgatcacagtcaaatagaagggcctgtgtaatgaaaatgtcagctggtctctgacaggtgagcttgaggtggagactttgagagaaccccagaactcctggttccagagacttgggctcttgggactttgttcccctgccctgccctgttcctgccacctgctgtccggccccaccccaggaagcattactctacagaagcatttccctggcacagtgtacgggctgagctcccaagagaggaacactcaaaatccagcttctcagcggtgcccagagccagcagcgccctggtcctcgtgtgtcccatttcagaatgaccaaggggaagtggaggacaaggaggacaagagaagcaaagggaggtagcgtccattaaagtatagcagagctcctgtcttcgctctggacgtcaaccctaggctatttgaatgtgcccagtagctgccaggtggcgggcactgggaagcagcctttcttggctcaagaccagcatctctgctctgtggctcacaagggagtggaggtcacaaaggaagacacagaaatggagcttggaaggctccgtctcttcccctctccagactaggaccttgtgtgtctcccaaggctttgacagttctaggtttgcttgcctcctcctcagagatctccattctccctcactcccaagtccttaccttccccccaggatatgaacggttccttgaagctctggcttaatgggtaacgtttagaaactgtggggattattccagctttttagctactcctgctaagattcacattccttccccgactctccctttttggacaattcagtacttattcagccttcattgaagggcaagcttaagaacaaaaatagtccttcacttttgtgcagagccctacgagatagtcttatagacgcttcatctcagccctgtgaacacgctatcattactccttctttcagagatgggctcgattctggaaacagtgttcaaaccttcattactttgttcccagcctcatcagtattctctgatcctgcatcacctactccccatcctacatcacctactccccactgatttcttctttaattctcatgactactgaggcatgagtgtgtaatcacatgagggaagtattcacagggagggaacatagccggctggggcttgcttttgtagggttattaggattagacaaggaatggggctgagctccattaattaatggctttttaaggagagaatttgagaaaatagacacatttaatctgatttggaaaatgtaccatgaatacatgtgtcaaaatatcacagtacttcactaaattaaggggaaaagagaacagaagacagacgggttcatacatatattccctgtgtcttgccatgtgaagtcctataccatctcttatgcgaggaaacctattcatcagggggcaaactagtgttccccacatgattttaatgctccagagctgtgagccaaaataaaccatgctcaacccggaaaaaaaaaaaaaaaa

SEQ ID NO: 23 - DNA, 703 bp, Homo sapiens
gcctggggtcacagggatgccgcggctcctccgcttgtccctgctgtggcttggactcctgctggttcggttttctcgtgaactgagcgacatcagcagtgccaggaagctgtgcggcaggtacttggtgaaagaaatagaaaaactctgcggccatgccaactggagccagttccgtttcgaggaggaaacccctttctcacggttgattgcacaggcctcggagaaggtcgaagcctacagcccataccagttcgaaagcccgcaaaccgcttccccggcccggggaagaggcacaaacccagtgtctacttcttgggaagaagcagtaaacagttgggaaatgcagtcactacctgagtataaggataaaaagggatattcaccccttggtaagacaagagaattttcttcatcacataatatcaatgtatatattcatgagaatgcattttttcagaagaaacgtagaaacaaaattaaaaccttaagcaatttgttttgggggcatcatccccaaagaaaacgcagaggatattcagaaaagtgttgtcttacaggatgtacaaaagaagaacttagcattgcatgtcttccatatattgattttaaaaggctaaaggaaaaaagatcatcacttgtaactaagatatactaaccatcttagaattttttctaacctaataaaagcttaatacattta

SEQ ID NO: 24 - protein, 5 aa, Artificial Sequence
  Description of Artificial Sequence Synthetic peptide
GGGGC